(19) United States
(12) Patent Application Publication
(10) Pub. No.: US 2007/0218495 A1
(43) Pub. Date: Sep. 20, 2007

(54) Methods, libraries and computer program products for gene silencing with reduced off-target effects

(75) Inventors: Amanda Birmingham, Lafayette, CO (US); Emily Anderson, Lafayette, CO (US); Angela Reynolds, Conifer, CO (US); Devin Leake, Denver, CO (US); Scott Baskerville, Louisville, CO (US); Yuriy Fedorov, Superior, CO (US); Jon Karpilow, Boulder, CO (US); William Marshall, Boulder, CO (US); Anastasia Khvorova, Boulder, CO (US)

Correspondence Address: KALOW & SPRINGUT LLP, 488 MADISON AVENUE, 19TH FLOOR, NEW YORK, NY, 10022, US

(73) Assignee: Dharmacon, Inc., Lafayette, CO (US)

(21) Appl. No.: 11/724,346

(22) Filed: Mar. 15, 2007

Related U.S. Application Data
(60) Provisional application No. 60/782,970, filed on Mar. 16, 2006.

(51) Int. Cl. C40B 40/08; C07H 21/02

(52) U.S. Cl. 435/6; 536/23.1

(57) ABSTRACT

The present invention provides methods, libraries and computer program products for selecting siRNA that reduce off-target effects and methods for gene silencing using these siRNAs. By comparing nucleotide sequences at positions 2-7 or 2-8 of the sense and/or antisense regions of candidate siRNAs to the 3′ UTR region of mRNAs, one can select siRNAs that have reduced off-target effects.

### CROSS-REFERENCE TO RELATED APPLICATION

This application claims the benefit of U.S. Provisional Application Ser. No. 60/782,970, filed Mar. 16, 2006. The entire disclosure of that application is incorporated by reference as if set forth fully herein.

### FIELD OF THE INVENTION

The present invention relates to RNA interference.

### BACKGROUND OF THE INVENTION

RNA interference (“RNAi”) refers to the silencing of the expression of a gene through the introduction of a RNA duplex into a cell. In RNAi, the RNA duplex is designed such that one strand (the antisense strand) has a region (the antisense region) that is complementary to a region of a target sequence, and the other strand (the sense strand) has a region (the sense region) that is complementary to the antisense strand. In mammals, RNAi requires the use of a small interfering RNA molecule (“siRNA”) that contains both an antisense region and a sense region. Use of longer molecules in mammals results in the undesirable interferon response.

One problem with applying RNAi techniques is that an siRNA that is directed against one particular target may silence another gene. This is referred to as an “off-target effect,” which has been observed to result in 1.5 to 5-fold changes in the expression of dozens to hundreds of genes by either transcript degradation or translation attenuation mechanisms. Off-target effects can occur from either the sense strand or the antisense strand and can occur when as few as 15 base pairs of complementarity exist between the siRNA and target. Jackson et al., (2003) “Expression profiling reveals off-target gene regulation by RNA,” Nat. Biotechnol. 21, 635-7.

Off-target gene silencing can present a significant challenge in the interpretation of large-scale RNAi screens for gene function and the identification and the use of optimal lead components for therapeutic applications. At one time, it was believed that off-target effects were due to overall identity of either strand of an siRNA duplex and a sequence other than the target. However, the inventors have determined that overall identity, i.e., based on all or most of the nucleotides in either the sense and/or antisense region being the same as or complementary to a region of a gene that is not being targeted, cannot very well predict off-target effects, except for near perfect matches.

One solution known to persons of ordinary skill for reducing off-target effects has been to use modifications of nucleotides at select positions within the duplex. Examples of these modifications are described in PCT application, PCT/US2005/011008, publication number WO 2005/097992 A2. However, modifications are not effective on all siRNA, can be expensive, and are not applicable to DNA-based RNAi (i.e. vector driven RNAi). Thus, there remains a need to develop other means to reduce off-target effects. The present invention is directed to this need.

### SUMMARY OF THE INVENTION

The present invention is directed toward reducing off-target effects in RNAi mediated gene silencing applications. Through the use of the methods, libraries and computer program products of the present invention, a person of ordinary skill can reduce the likelihood that an siRNA that is selected will have undesirable levels of off-target effects.

According to a first embodiment, the present invention provides a method for selecting an siRNA for gene silencing in humans, said method comprising: (a) selecting a target gene, wherein the target gene comprises a target sequence; (b) selecting a candidate siRNA, wherein said candidate siRNA comprises 18-30 nucleotide base pairs that form a duplex comprised of an antisense region and a sense region and said antisense region of said candidate siRNA is at least 80% complementary to a region of said target sequence; (c) comparing a sequence of the nucleotides at positions 2-7 of said antisense region of said candidate siRNA to a dataset wherein said dataset comprises the nucleotide sequences of the 3′ UTR regions (3′ untranslated regions) of a set of human RNA sequences; (d) comparing a sequence of the nucleotides at positions 2-7 of said sense region of said candidate siRNA to said dataset; and (e) selecting said candidate siRNA as a siRNA for gene silencing, if said sequence of the nucleotides at positions 2-7 of said antisense region are 100% complementary to sequences within fewer than 2000 3′ UTRs of mRNA within said dataset and/or the nucleotides at positions 2-7 of said sense region are 100% complementary to sequences within fewer than 2000 3′ UTR regions of mRNA within the dataset. Two thousand (2000) 3′ UTRs represents approximately 8.5% of the known 23,500 known human NM 3′ UTR sequences. As databases change in size and differ across organisms it may be useful to set the limit as 5%-15% of the known sequences in a given dataset. Preferably for any organism considered, there are at least 5,000, more preferably at least 10,000 known sequences in a dataset when the method is applied. For humans it was observed that based on the known number of sequences, the set of seeds that appear in fewer than 2000 3′UTRs excludes essentially all of the seed sequences that do not contain the CG nucleotide. Accordingly, although there may be more than 2000 3′UTRs that contain certain seeds with the CG dinucleotide, there are substantially no seeds that appear in fewer than 2000 3′UTRs that do not contain this dinucleotide.

Positions 2-7 may be referred to as a hexamer sequence. Alternatively, one may focus on positions 2-8, which may be referred to as a heptamer sequence. The nucleotide sequence of the siRNA that is compared to the 3′ UTR may be referred to a “seed sequence,” regardless of whether positions 2-7 or 2-8 of the sense or antisense strand are examined for complementarity to the 3′ UTR region. The siRNA that is selected for gene silencing may be introduced into a cell and used to silence the target gene while causing a relatively low level of off-target effects. When performing the above-described method, one may start with one candidate siRNA, a plurality of siRNAs, or all possible siRNAs that contain antisense regions that are complementary to a region of a target sequence. Preferably the antisense region is at least 80% complementary to a region of the target sequence and more at least 90% and most preferably 100% complementary to a region of the target sequence.

In a second embodiment, the present invention provides a method for converting an siRNA having desirable silencing properties, yet undesirable off-targeting effects, into an siRNA that retains the silencing properties (or has a functionality that is decreased by no more than 10%, more preferably no more than 5% and most preferably no more than 3%), yet has the lower levels of off-target effects describe above. The method comprises comparing the sequence of the seed of the siRNA with a database comprising low frequency seeds (or 3′ UTRs that may be searched according to the frequency of the hexamer or heptamer sequences) and identifying one or more single nucleotide changes that could be incorporated into the seed sequence of the siRNA such that the seed sequence is converted to a low frequency sequence without losing silencing activity. Unless otherwise specified, a low frequency seed is a sequence that appears in fewer than 2000 known human 3′ UTR regions when a siRNA is directed to a human target gene. A seed sequence that appears more than one time in a 3′ UTR is counted as only a single occurrence for the purpose of the present invention. The aforementioned silencing activity could be determined empirically and/or predicted through rational design criteria as described below.

In a third embodiment, the present invention provides a method of designing a library of siRNA sequences. The method comprises collecting siRNA sequences of at least 100 siRNAs that target at least 25 different genes, wherein said siRNA sequences comprise 18-30 bases, and at least 25% of the siRNA sequences have a hexamer sequence at positions 2-7 of an antisense sequence selected from reverse complement of the sequences of the group consisting of the sequences in Table V below.

The library could in its simplest form be created by identifying a set of candidate siRNA for a plurality of target sequences, and manually typing them into a computer database such that on average at least one of every four siRNAs that are input contain a seed sequence that is the reverse complement of a sequence identified in Table V. Preferably the siRNA within the library all have a selected level of functionality, which may for example be determined by trial and error or may be predicted to be among the most functional through bioinformatics techniques such as those described in U.S. Ser. No. 10/714,333 or PCT/US04/14885. When the library contains both siRNA with seed sequences that are the reverse complement of those within Table V and siRNA with seed sequence that are not the reverse complement of those within Table V, preferably the siRNA that have seed sequences that are the reverse complement of the hexamers in Table V are denoted or otherwise tagged as containing such a sequence.

In a fourth embodiment, the present invention provides a library of siRNA sequences, said library comprising a collection of siRNA sequences of at least 100 siRNAs that target at least 25 different genes, wherein said siRNA sequences comprise 18-30 bases, and at least 25% of the siRNA sequences have a hexamer sequence at positions 2-7 of an antisense sequence selected from the group consisting of the reverse complement of the sequences in Table V below. This library may be populated through the entry of data into an appropriate computer program. As persons of ordinary skill are aware, the computer program will include code for receiving data corresponding to nucleic acid sequences and for searching among this type of data. Preferably, the library also contains a means to differentiate between ORF, 5′ UTR and 3′ UTR (and other untranslated sequences). Further, although positions 2-7 of the antisense strand are referenced above, this information is understood to refer implicitly to positions 12-18 of the opposite strand in a 19-mer (or corresponding positions in a strand of a different length e.g., positions 22-28 in a 29-mer).

In a fifth embodiment, the preset invention provides a computer program product for use in conjunction with a computer system, the computer program product comprising a computer readable storage medium and a computer program mechanism embedded therein, the computer program mechanism comprising: (a) an input module, wherein said input module permits a user to identify a target sequence; (b) a database mining module, wherein said database mining module is coupled to said input module and is capable of searching a siRNA database comprised of at least 100 siRNA sequences that target at least 25 different genes, wherein each of said siRNA sequences comprises 18-30 bases, and (c) an output module, wherein said output module is coupled to said database mining module and said output module is capable of providing to said user an identification of one or more siRNA sequences from said database where each siRNA that is identified comprises an antisense sequence that is at least 80% complementary to a region of said target sequence and at least 25% of the siRNA sequences identified from said database have a hexamer sequence at positions 2-7 of said antisense sequence selected from the group consisting of the reverse complement of sequences in Table V below. In some embodiments, at least 25% of the siRNA also have a hexamer sequence at positions 2-7 of the sense sequence selected from the group consisting of the reverse complement of sequences in Table V.

### BRIEF DESCRIPTION OF THE FIGURES

FIG. 1 is a representation of a microarray analysis that identifies off-targeted genes.

FIGS. 2A and 2B are representations of the results of an analysis that shows that maximum sequence alignment fails to predict accurately off-targeted gene regulation by RNAi. The sense (top) and antisense (bottom) sequences of each siRNA were aligned separately to the sequences of their corresponding 347 experimentally validated off-targets and a comparable number of control untargeted genes to identify the alignments with the maximum percent identity. The number of alignments in each identity window was then plotted for the off-targeted (black) and untargeted (white) populations.

FIGS. 3A-3D are representations of a systematic single base mismatch analysis of siRNA functionality.

FIG. 4 is a representation of the variations of Smith-Waterman scoring parameters that fail to improve the ability to distinguish off-targets from untargeted genes.

FIGS. 5A-5C are bar graphs that show that exact complementarity between the siRNA seed sequence and the 3′ UTR (but not 5′ UTR or ORF) distinguishes off-targeted from untargeted genes.

FIG. 6 is a bar graph that demonstrates that the seed sequence association with off-targeting is not due to 3′ UTR length.

FIGS. 7A and 7B. FIG. 7A is a graph of the frequency of all possible heptamer sequences in a collection of human 3′ UTRs. FIG. 7B is a graph of the frequency of all possible hexamer sequences in a collection of human 3′ UTRs. While the frequency of some seeds is very low, others are quite high. The distribution of a subset of the heptamer and hexamer sequences is shown.

FIGS. 8A and 8B. FIG. 8A is a representation of the distribution of seeds by frequency in 3′ UTRs for Refseq 15 Human NM 3′UTRs, from lmh_analysis.xls. FIG. 8B is a representation of the distribution of seed by frequency in 3′ UTRs for Refseq 17 Rat NM 3′ UTRs, from SeedThresholdForMouseRat.xls.

### DETAILED DESCRIPTION

The present invention provides methods for reducing off-target effects during gene silencing and methods for selecting siRNA for use in these applications. The present invention also provides libraries and computer program products that assist in increasing the likelihood that siRNA will have reduced off-target effects.

The inventors have discovered that the number of off-targets generated by an siRNA can be limited by choosing an siRNA that has a sense and/or antisense seed sequence that has limited numbers of complementary sequences in the 3′ UTR sequences of messenger RNAs of the target genome. As the frequency at which a seed match appears in the population of 3′ UTRs of a genome is predictive of the number of off-targets, it is possible to select for siRNA that have fewer off-targets.

To that end, according to a first embodiment the present invention comprises a method for selecting an siRNA for gene silencing in a human cell. The method comprises: (a) selecting a target gene, wherein the target gene comprises a target sequence; (b) selecting a candidate siRNA, wherein said candidate siRNA comprises 18-30 nucleotide base pairs that form a duplex comprised of an antisense region and a sense region and said antisense region of said candidate siRNA is at least 80% complementary to said target sequence; (c) comparing a sequence of the nucleotides at positions 2-7 of said antisense region of said candidate siRNA to a dataset wherein said dataset comprises the nucleotide sequences of the 3′ UTRs of a set of human RNA sequences; (d) optionally, comparing a sequence of the nucleotides at positions 2-7 of said sense region of said candidate siRNA to said dataset; and (e) selecting said candidate siRNA as an siRNA for gene silencing, if said sequence of the nucleotides at positions 2-7 of said antisense region and of said sense region are each complementary to hexamer sequences that appear in the 3′ UTRs of fewer than 2000 mRNA. A similar method can be devised based on the frequency of heptamer sequences. However, because there are four times as many possible heptamer sequences, each heptamer sequence will occur on average less frequently that each hexamer sequence. Accordingly, one, could look to select siRNA that have heptamer sequences at positions 2-8 that appears in fewer than 500 3′ UTRs of human mRNA.

One may omit step (d) when employing this method, in which case during step (e), one would only compare the seed sequence within the antisense region to the 3′ UTR regions. Preferably, step (d) is not omitted unless the duplex will be modified (e.g. through chemical modifications) or contain another cause of strand bias that reduces the likelihood that the sense strand can induce RNAi and thus is rendered essentially incapable of generating undesirable levels of off-target effects.

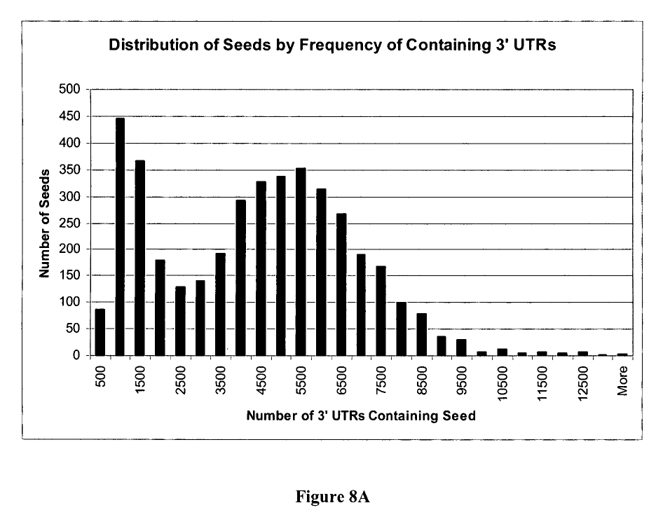

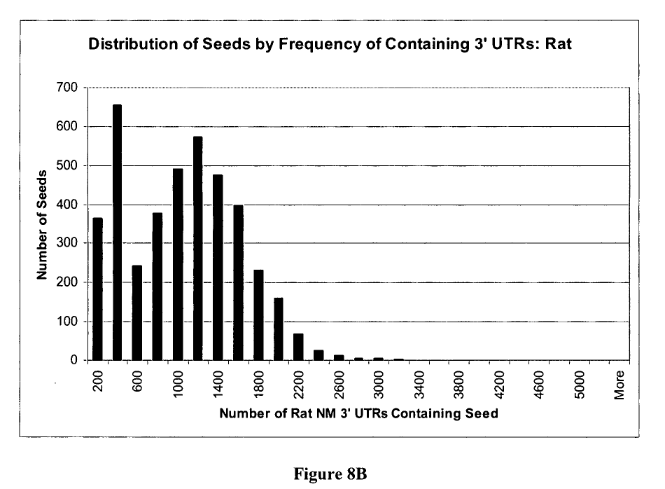

The number of 3′ UTRs in which seed sequences appear that is selected as the cut off for an organism is selected based on the discovery that the appearance of seed sequences in 3′ UTRs forms a bimodal distribution. As described more fully in example 4 below and FIGS. 8A and 8B, hexamer and heptamer sequence do not occur randomly in 3′ UTRs. Instead, when one examines the distribution of seeds by frequency of 3′UTRs that contain them and bins the number of times that seed sequences appear in different known 3′ UTRs for a given species, a bimodal distribution is observed.

When the 4096 possible hexamer seeds are binned by the number of human NM 3′ UTRs in which they appear, the resulting histogram shows a clear bimodal distribution. The sharp secondary peak at the left of the histogram represents a distinct population of 3′ UTRs with low frequency seeds. This low frequency may be due to the ubiquitous presence of the CG dinucleotide in these seeds, as the CG dinucletoide is rare in mammals. For humans, the cut off frequency between the two nodes is located at approximately 2000 3′ UTRs (see FIG. 8A), which leaves approximately 8.5% of the known 3′UTRs to the left of this point and thus qualifies the seeds contained in those regions as low frequency. FIG. 8A was produced from two groups of seeds, those containing CG (left) and those not containing CG (right). When the two distributions are examined individually, the non-CG containing seeds do not begin to appear in measurable number until about 2500 on the x-axis. Thus, the cut off was selected to exclude seed sequences that appear with that frequency and higher.

For the rat, this point is approximately 600 for known sequences (see FIG. 8B), which renders approximately 7.5% of the known 3′UTRs to the left of this point on a bimodal distribution. For mouse, not shown, the corresponding point between the two nodes renders approximately 11.0% of the sequences to be low frequency seeds. Within any given species, one would expect that when the frequency of the seed sequences is calculated and plotted on a graph similar to those of FIGS. 8A and 8B, between 5% and 15% of the 3′UTRs would be represented by points to the left of the first appearance of significant numbers of sequences in the second node.

With respect to implementing the present invention, and as persons skilled in the art are aware, if one assumes 100% complementarity and one knows the length of the duplex, by examining one strand, information is implicitly provided about the other strand. Thus in a 20-mer duplex, information about positions 2-7 of the antisense strand may be learned by focusing on positions 14-19 of the sense strand.

The Datasets

The phrase “dataset” and term “database” are used interchangeably and refer to sets or libraries of sequences. The sequences of a database can represent the total collection of e.g., 3′ UTRs of an organism's genome, or expressed 3′ UTRs for e.g. a particular cell type. Accordingly, databases include but are not limited to those that contain the complete or cell specific mRNA sequences or 3′ UTR sequences e.g., GenBank or Pacdb (http://harlequin.jax.org/pacdb/). Such databases can be used to select targets and candidate siRNAs. Additionally, cDNA databases preferably generated using poly-dT primers can be used to select targets and candidate siRNAs. Alternatively or additionally, databases may compromise siRNA sequences. These sequences may be defined by parameters that include but are not limited to length, target sequences, species and predicted or empirical functionality. The siRNA sequences may also have data associated with them that identify gene(s) that they target.

The data may be stored on relational databases or file based databases. Examples of relational databases include but are not limited to Sequel Server, Oracle, and MySeql. An example of a file-based database includes but is not limited to File Maker Pro.

The Target Gene

A “target gene” is any gene that one wishes to silence. As persons skilled in the art are aware, typically siRNAs silence a target gene by becoming associated with RISC (the RNA Induced Silencing Complex) and then cleaving or inhibiting the translation of the target gene messenger RNA (“mRNA”). The mRNA comprises both a coding sequence, which will be translated into a protein or polypeptide, and a 3′ UTR (3′ untranslated region). The mRNA may contain other areas as well, including a 5″ UTR, and/or a tail (e.g., poly A tail). The target gene may be selected based on the desire to study or to knockdown (i.e., reduce expression of) that gene. The “target sequence” is, unless otherwise specified, a portion of the mRNA that codes for a protein.

The siRNA

After a gene is selected, at least one candidate siRNA is examined, and preferably a plurality of candidate siRNAs are examined. A candidate siRNA is any siRNA that contains an antisense region that is at least 80%, and preferably 100% complementary to a portion of a target sequence. As persons skilled in the art are aware, one may look at the sequence of the antisense region or the sequence of the sense region, which will, assuming 100% complementary between the antisense region and sense region, provide information about the other region. (The principles of reverse complementary are well known to persons of ordinary skill and are based on standard A-T(or U) and G-C base pairing and the anti-parallel nature of nucleic acid duplexes.)

When working in mammals such as humans, chimpanzees, rats, mice, horses, sheep, goats, cows, dogs, cats, etc., preferably the siRNA comprises 18-30 base pairs, more preferably 19-25 base pairs, even more preferably 19-24 base pairs and most preferably 19-23 base pairs. Preferably the antisense region is at least 80% complementary to a region of the target sequence, more preferably at least 90% complementary to a region of the target sequence, even more preferably at least 95% complementary to a region of the target sequence and most preferably 100% complementary to a region of the target sequence. Unless otherwise specified, the antisense region and the region of the target sequence are presumed to be 100% complementary to each other.

The base pairs of an siRNA will form a duplex comprised of an antisense region and a sense region. A candidate siRNA may be comprised of either two separate strands, one of which comprises the antisense region (the antisense strand) and the other of which comprises the sense region (the sense strand). The candidate siRNA may also comprise one long strand, such as a hairpin siRNA. Alternatively, the candidate siRNA may comprise a fractured or nicked hairpin that is a duplex comprised of two strands, one of which contains all of the sense region and part of the antisense region, while the other strand contains part of the antisense region. Similarly, a fractured or nicked hairpin may be a duplex comprised of two strands, one of which contains all of the antisense region and part of the sense region, while the other strand comprises part of the sense region. These types of hairpin molecules are also described in pending U.S. patent application Ser. No. 11/390,829, which was filed on Mar. 28, 2006 and published as US 2006-0223777 A1 on Oct. 5, 2006.

Within the duplex of the siRNA, the antisense region and the sense region are preferably at least 80% complementary to each other, more preferably at least 90% complementary to each other, even more preferably at least 95% complementary to each other and most preferably at least 100% complementary to each other. Unless otherwise specified, the antisense region and the sense region are presumed to be 100% complementary to each other.

The candidate siRNA may have blunt ends or overhangs on either the 5′ or 3′ ends. If any overhangs are present, preferably they will be 1-6 base pairs in length and on the 3′ end of either or both of the antisense strand or sense strand. More preferably, the overhangs will be 2 base pairs in length on the 3′ end of the antisense or sense strand. If the siRNA is a hairpin or fractured hairpin molecule, it will also contain a loop structure.

The candidate siRNA may have modifications, such as 5′ phosphate groups, modifications of the 2′ carbon of the ribose sugars, and internucleotide modifications. Exemplary modifications include 2′-O-alkyl modification (e.g., 2′-O-methyl, 2′-O-ethyl, 2′-O-propyl, 2′-O-isoproyl, 2′-O-butyl), 2′fluoro modifications, 2′ orthoester modifications, and internucleotide thio modifications. The modifications may be included to increase stability and/or specificity.

Modifications can be added to siRNA to enable users: (1) to apply the invention to one strand; or (2) to enhance the efficiency of the invention. As described in U.S. patent application Ser. No. 11/019,831, publication no. US2005-0223427A1 chemical modifications can be added to enhance specificity. Thus, for example, addition of a 5′ phosphate group on the first antisense nucleotide, and 2′ O-alkyl modifications (e.g., 2′ O-methyl) on the first sense nucleotide and the second sense nucleotide eliminate the ability of the sense strand to enter RISC, and thus would allow users to confine the method of the invention to the antisense strand. Alternatively, the method of the invention can be applied to both strands to identify siRNA with desirable traits, and subsequently modifications can be added to both strands (e.g., (1) a 5′ phosphate group on the first antisense nucleotide, and 2′ O-alkyl modification (e.g., 2′ O-methyl) on the first 5′ sense nucleotide, the second 5′ sense nucleotide, the first 5′ antisense nucleotide and the second 5′ antisense nucleotide; or (2) a 5′ phosphate group on the first 5′ antisense nucleotide, and 2′ O-alkyl modification (e.g., 2′ O-methyl) of the first 5′ sense nucleotide, the second 5′ sense nucleotide and the second 5′ antisense nucleotide) to minimize off-targets further. When modifications are present, all nucleotides that are not specifically identified as having a modification are preferably unmodified, i.e., they have 2′OH groups on their ribose sugars. Thus, the presence of modifications such as 2′ modifications on one or both strands does not preclude application of the current invention. In fact, because certain modifications may reduce off-target effects, but not to the degree desired, in some instances it is advantageous to apply the current invention to both strands of a duplex regardless of whether there are any chemical modifications or other bases for strand bias.

The phrase “first 5′ sense nucleotide” refers to the 5′ most nucleotide of the sense region, and thus this nucleotide would be part of the duplex formed with the antisense region. The phrase “second 5′ sense nucleotide” refers to the next 5′ most nucleotide of the sense region. The second 5′ sense nucleotide is immediately adjacent to and downstream (i.e. 3′) of the first 5′ sense nucleotide, and thus would also be part of the duplex formed. The phrase “first 5′ antisense nucleotide” refers to the 5′ most nucleotide of the antisense region. The phrase “second 5′ antisense nucleotide” refers to the next 5′ most nucleotide of the antisense region. The second 5′ antisense nucleotide is immediately adjacent to and downstream of the first 5′ antisense nucleotide. The first 5′ antisense nucleotide and second 5′ antisense nucleotide are also each part of the duplex formed with the sense region. Thus, any 5′ overhangs do not affect the definition of the aforementioned first or second 5′ nucleotides.

The nucleotides within each region may also be referred to by their positions relative to the 5′ terminus of that region. Thus, the first 5′ antisense nucleotide is located at position 1 of the antisense region, the second 5′ antisense nucleotide is located at position 2 of that region, the third 5′ antisense nucleotide is located at position 3 of that region, the fourth 5′ antisense nucleotide is located at position 4 of that region, the fifth 5′ antisense nucleotide is located at position 5 of that region, etc. A similar convention can be used to identify the nucleotides of the sense region; however, note that in a duplex of 19 base pairs, position 1 of the sense region will appear opposite position 19 of the antisense region. Unless otherwise specified the hexamer and heptamer sequences that are examined in the context of the present invention refer to positions 2-7 and 2-8, respectively of the antisense and/or sense regions of the siRNA.

Previous investigations known to persons of ordinary skill in the art have suggested that off-target effects could be eliminated by minimizing the overall levels of complementarity between an siRNA and unintended targets in the genome of interest. The inventors have demonstrated that this technique is not viable (see Birmingham et al., (2006) “3′ UTR seed matches, but not overall identity, are associated with RNAi off-targets” Nature Methods 3:199-204) and instead, have identified key parameters that allow RNAi users to minimize off-target effects. First, as shown in Example 1, it was observed that the 3′ UTR of off-targeted genes frequently have one or more sequences that are the reverse complement of the seed sequence of a siRNA. Second, as shown in Example 2, the inventors observed that the frequency at which all hexamers and/or heptamers appear in the 3′ UTR sequences of any given genome (e.g. human, mouse, and rat genomes) varies considerably. It was also observed that a correlation exists between the number of off-targets generated by a particular siRNA, and the frequency at which the reverse complement of the seed sequence of the siRNA appears in the 3′ UTRs of the genome. These three previously undocumented observations have allowed the inventors to construct the novel method for minimizing off-target effects described herein.

When seeking to reduce off-target effects, preferably one focuses on positions 2-7 of the antisense region and/or sense region or positions 2-8 of the antisense region and/or sense region of a candidate siRNA. It is preferable to consider both strands because either strand could in theory generate an off-target effect. Focusing on a smaller number of positions may lead to false positive matches and focusing on a greater number of positions may lead to false negative results. In addition, when applying the method of the present invention, it is important to focus on the seed sequence of the siRNA that will result after any Dicer or other processing. Thus, if for example, Dicer cleaves a 30 base pair, double stranded RNA (“dsRNA”) into two very different ˜23 base pair duplexes, depending on whether the cleavage began at the 5′ end of the sense strand (3′end of the antisense strand), or at the 5′ end of the antisense strand (3′end of the sense strand), as a result, the nucleotides that are the second through the seventh (or eighth) nucleotides of the antisense strand might be different. Accordingly, it is important to apply the method by selecting candidate siRNA that either will not be cleaved, e.g., are shorter than 24 base pairs, or to apply the method to what will be the sequences after cleavage of one or both ends. Thus, in some embodiments, it is preferably for the siRNA to contain 18-24 bases.

As noted above according to one embodiment of the present invention, one examines positions 2-7 or 2-8 of the antisense region and/or positions 2-7 or 2-8 of the sense region of a candidate siRNA and compares the sequence of the nucleotides located at those positions to the dataset containing sequences from the 3′ UTRs of mRNA of for example, a genome (e.g. a human genome 3′ UTR dataset) to determine whether complementary exists in one or more instances. In some embodiments, preferably, the dataset comprises the 3′ UTRs of at least 1500 mRNA sequences, more preferably of at least 2000 mRNA sequences, and even more preferably of at least 3000 mRNA. In some embodiments, the 3′ UTR regions of all known mRNA for a species or cell type are within the dataset (e.g. HeLa cells, or MCF7 cells). Preferably, the dataset is also species specific. In some embodiments, when trying to reduce off-target effects in cells expressing human genes, the dataset comprises a sufficiently large set of 3′ UTR regions of human mRNA, if not all known such regions.

After one examines positions 2-7 or positions 2-8 of the antisense region and/or the sense region of a candidate siRNA or collection of siRNA, one may select desirable siRNA based on the frequency of the seed matches in (i.e. instances of complementarity to) the 3′ UTR of e.g. the mRNA dataset. siRNA, for example, can be selected on the basis of having seed sequences that are complementary to sequences in fewer than about 2000 3′ UTRs, more preferably fewer than about 1500, even more preferably, fewer than about 1000 and even most preferably, fewer than about 500 sequences in 3′ UTR regions. Note that a sequence may appear two or more times within a 3′ UTR of a given gene. In these cases each additional occurrence would not be considered an additional match.

Although not wishing to be bound by any one theory, it is postulated that the advantage of using siRNA that have low frequencies in the 3′ UTR regions is due to the relatively limited amount of RISC in a cell. RISC is an integral part of gene silencing in mammals, and RISC may be guided to a target by at least two means. First, RISC may be guided to a target when there is complementarity of a region of the siRNA to the target sequence, typically a region of at least 18 nucleotides. Second, RISC may be guided to another RNA molecule when there is complementarity between positions 2-7 or 2-8 of the antisense region or positions 2-7 or 2-8 of the sense region of the siRNA and a sequence in the 3′ UTR of another molecule.

When the sequence at positions 2-7 or 2-8 of a candidate siRNA appears relatively infrequently in the 3′ UTRs of the set of mRNA for a species, low levels of off-targeting occurs because there are a limited number or potential off-targeted genes that contain seed molecules.

There are 4096 (4⁶) different sequences for the six nucleotides from positions 2-7, and 16,384 (4⁷) different sequences for the seven nucleotides from positions 2-8 assuming canonical bases, i.e., A, C, G, U. Thus, the method for comparing the candidate siRNA to a dataset comprising 3′ UTRs may be performed most easily by a computer algorithm. The use of computer algorithms to manipulate and to select nucleotide sequences is well known to persons of ordinary skill in the art.

The dataset could be organized by inputting all or a sufficiently large set of mRNA, including their 3′ UTRs. Then one, a plurality, or all candidate siRNAs of a given size or multiple sizes could be compared against the dataset to determine the number of times that the antisense seed sequence and/or the sense seed sequence are complementary to 3′ UTR sequences in the dataset. One could weed out siRNA that do not have low frequency seeds. Alternatively, one could create a dataset of 3′ UTRs, search for the number of times that each stretch of 6 or 7-mers repeat and then for each unique 6 or 7-mer maintain the information for the number of times that it repeats. The result of the frequency of hexamers based on human 3′ UTRs in RefSeq Version 17 from the NCBI database is identified in Table V. The seed sequences of the candidate siRNA could, for example, then be compared against this set of information to look for complementary sequences and thus determine the likelihood of off-target effects.

The datasets of the present invention may be organized into specific libraries. For example, one may create a library of at least 100 different siRNAs that target at least 25 different genes (e.g., an average of four siRNA per target) where at least 25% of the siRNA have a seed sequence selected from Table V. Preferably there are at least 200 different siRNA, more preferably at least 500 different siRNA, even more preferably at least 1000 different siRNA, even more preferably at least 2000 different siRNA, even more preferably at least 5000 different siRNA. Further, preferably the library contains siRNA that target at least 50 different genes, more preferably at least 100 different genes, even more preferably at least 200 different genes, even more preferably at least 400 different genes, even more preferably at least 500 different genes, and even more preferably at least 1000 different genes. A more comprehensive library would contain siRNA that target the entire genome. For example, such a library may contain 100,000 siRNAs for about 25,000 different genes (four siRNAs per gene).

In some embodiments, preferably at least 40%, more preferably at least 50%, even more preferably at least 80%, even more preferably at least 90% and most preferably 100% of the siRNA have a seed sequence that is the reverse complement of a sequence selected from Table V.

The method for selecting siRNA of the present invention may be used in combination with methods for selecting siRNA based on rational design to increase functionality. Rational design is, in simplest terms, the application of a proven set of criteria that enhance the probability of identifying a functional or hyperfunctional siRNA. These methods are for example described in commonly owned WO 2004/045543 A2, published on Jun. 3, 2004, U.S. Patent Publication No. 2005-0255487 A1, published on Nov. 17, 2005, and WO 2006/006948 A2 published on Jan. 19, 2006 the teachings of which are incorporated by reference herein. When selecting siRNA for the aforementioned libraries, one may apply rational design criteria to a set of candidate siRNAs, and then weed out some or all sequences that do not meet the aforementioned seed criteria. Thus, the seed criteria are a filter applied to rational design criteria. Alternatively, one could weed out some or all sequences that do not satisfy the seed criteria, and then apply rational design criteria.

Combining the method of the invention with siRNA selected by rational design as described above may allow users to simplify the application of the method by focusing on the seed sequence of the antisense strand. Rationally designed siRNA are (in part) selected on the basis that the antisense strand of the duplex (i.e. the strand that is complementary to the desired target) is preferentially loaded into RISC. For that reason, off-targets of rationally designed siRNA are predominantly the result of the 3′ UTR matches with the seed sequence of the antisense strand. Therefore, in cases where rationally designed siRNA having an antisense strand bias are being used, it is possible to confine the method of the invention to the antisense strand alone, and ignore possible off-target contributions by the sense strand.

The siRNA selected according to the present invention may be used in both in vitro and in vivo applications.

The siRNA used in connection with the present invention may be synthesized and introduced into a cell. Methods for synthesizing siRNA of desired sequences are well known to persons of ordinary skill in the art. These methods include generating duplexes and/or unimolecular molecules by chemical synthesis, enzymatic synthesis, or expression vectors of siRNA or shRNA.

In another embodiment, the invention provides a method for converting an siRNA having desirable silencing properties, yet undesirable off-targeting effects, into an siRNA that retains the silencing properties, yet has fewer off-targets. The method comprises comparing the sequence of the seed of the siRNA(s) with a database comprising low frequency seeds and identifying one or more single nucleotide changes that could be incorporated into the seed sequence of the siRNA such that the frequency of the seed is converted from a moderate or high frequency, to a low frequency, without losing silencing activity. In one non-limiting example of this method, highly functional siRNA containing an sense seed of 5′-AGGCCG, 5′-ACCCCG, or 5′-ACGCCT (seed frequencies of 2376, 2198, and 2001 based on all human NM 3′ UTRs derived from NCBI RefSeq 15) can be converted to a low frequency seed (5′-ACGCCG, 472 appearances) by altering a single nucleotide, thus generating the new, low frequency seed sequence. A “low frequency seed” refers to a sequence of bases that appears relatively infrequently in the 3′ UTR region of mRNAs, e.g., appears in equal to or fewer than about 2000 3′ UTR regions, more preferably fewer than about 1500 3′ UTR regions, even more preferably, fewer than about 1000 3′ UTR regions, and most preferably fewer than about 500 times in 3′ UTRs.

The present invention also provides a method for designing a library of siRNA sequences. By having a library of siRNA sequences, a person of ordinary skill has readily available a set of siRNA that have been pre-screened to, for example, have a reduced level of off-target effects. In one embodiment the library contains sequences of at least 100 siRNAs that target at least 25 different genes. Larger databases such as those described above are also within the embodiment.

The sequences within the library may be for one or both strands of an siRNA duplex that is 18-30 base pairs in length. Because of standard AU, GC base pairing it is not necessary to have both strands in the database. When a library has a plurality of siRNA for a given gene, a user may use individual sequences from the plurality or use them in a pool. Thus, by way of example, a user may select a highly functional siRNA such as that determined by Formula X of PCT/US04/14885 and filter those sequences by applying a low frequency seed siRNA criterion, which may for example, be any siRNA with a seed sequence that is the reverse complement of a sequence that is identified in Table V, or it may be an siRNA with the lowest seed frequency for the target, or it may an siRNA with the lowest seed frequency that is among the siRNA that have the two, three, four, five, six, seven, eight, nine, or ten highest predicted functionalities (or empirical functionalities, i.e., gene silencing capabilities if known). Alternatively, one may use pools of two, three, four, five, six etc., siRNA that have low if not the lowest seed frequencies. Still further one could combine pools of three, four, five, six, etc. siRNA for a target wherein within each pool one or more are selected based on functionality and one or more are selected based on seed frequency.

In Table V below is a list that represents hexamer nucleotide sequences that occur at least once in fewer than 2000 known human NM 3′ UTRs. There are 1081 hexamer sequences in the list. As noted above, the 4096 possible hexamers are not uniformly distributed in human 3′ UTRs, instead showing a distinct bimodal distribution including a population of low-frequency hexamers (as defined above). The inventors have demonstrated that siRNAs whose seeds occur infrequently in 3′ UTRs produce significantly fewer off-targets than those whose seeds occur at higher frequencies. The use of “T” in the table is by convention in most databases. However, it is understood as referring to a Uracil in any RNA sequence, including any siRNA sequence.

Additionally, it is desirable to create a library with a minimal percentage of siRNA sequences that have low seed frequencies. Although it may be preferable for most or all sequences to have low seed frequencies, that is not always practical for a given target gene, and other considerations such as functionality are important to consider. Thus, preferably on average at least one of every four siRNA sequences has a low frequency seed sequence, more preferably on average at least two of every four siRNA sequences has a low frequency seed sequence, even more preferably on average at least three of every four siRNA sequences has a low frequency seed sequence. In some embodiments at least one siRNA for each target contains a low frequency if not the lowest frequency seed sequence. Table V identifies the 1081 seed sequences that occur in the fewest 3′ UTRs. Also included in the table under the heading “distinctnmutr3” is the number of 3′ UTRs in which a given low frequency seed sequence appears.

Given the presentation of Table V, a person of ordinary skill could create a database by comparing the seed sequences of a plurality of siRNA to the sequences on Table V and inputting those siRNA into a searchable database if those siRNA contain the seed frequency below a requisite level. The person of ordinary skill may also include information about the functionality of the siRNA as well as its targets. Preferably, the library is searchable through computer technology and contains a mechanism for linking the sequence data with e.g., target data and/or seed frequency.

The libraries of the present invention may, for example, be located on a user's hard drive, a LAN (local area network), a portable memory stick, a CD, the worldwide web or a remote server or otherwise, including storage and communication technologies that are developed in the future.

The computer program products of the present invention could be organized in modules including input modules, database mining modules and output modules that are coupled to one another. In some embodiments, the modules may be one or more hardware, software or hybrid residing in or distributed among one or more local or remote computers. The modules may be physically separated or together and may each be a logic routine or part of a logic routine that carries out the embodiments disclosed herein. The modules are preferably accessible through the same user interface.

The software of the present invention may, for example, run on an operating system at least as powerful as Windows 2000.

The computer program may be written in any language that allows for the input of a sequence and searching within a dataset for an siRNA that targets the sequence based on complementarity or identity. For example, the computer program product may be in C#, Pearl or LISP. The program may be run on any standard personal computer or network system. Preferably the computer is of sufficient power to quickly mine large datasets, such as those of the present invention, e.g., 2.33 GHz, 256 RAM and 80 Gb.

The input module will thus be accessible to a user through a user interface and permit a user to select a target gene by for example, name, accession number and/or nucleotide sequence. The input module may offer the user the ability to request the format of the output, and the content of the output, e.g., request the lowest frequency seed to be output and/or the lowest frequency with a set of the highest functional siRNAs, e.g., the siRNA whose functionality is predicted to the highest by a set of rational design criteria.

The input module may then convert the inputted data into a standard syntax that is sent to the database mining module. The database mining module then searches a database containing a set of siRNA that are either complementary to or similar to a region the target depending on whether sense or antisense information is input. The database mining module then transmits the result to the output module, which either saves the results and/or displays them on a user interface. The computer program product may be configured such that the database mining module searches within a database that is part of the computer program product, and/or configured to mine a stand alone database.

The computer program product, as well as the library and methods described herein may be used to assist persons of ordinary skill in the art to identify siRNA with an increased likelihood of having reduced off-target effects.

The computer program product may be run on any standard personal computer that has sufficient power capabilities. As persons of ordinary skill in the art are aware, a more powerful computer may be able to manipulate larger amounts of data at a faster rate. Exemplary computers include but are not limited to personal computers currently sold by IBM, Apple, Dell and Gateway.

Having described the invention with a degree of particularity, examples will now be provided. These examples are not intended to and should not be construed to limit the scope of the claims in any way. Although the invention may be more readily understood through reference to the following examples, they are provided by way of illustration and are not intended to limit the present invention unless specified by and in the claims.

### EXAMPLES

General Methods

siRNA Synthesis. siRNA duplexes targeting human PPIB (NM_{—}000942), MAP2K1 (NM_{—}002755), GAPDH (NM_{—}002046), and PPYLUC (U47295), were synthesized with 3′ UU overhangs using 2′-ACE chemistry Scaringe, S. A. (2000) “Advanced 5′-silyl-2′-orthoester approach to RNA oligonucleotide synthesis,” Methods Enzymol. 317, 3-18; Scaringe, S. A. (2001) “RNA oligonucleotide synthesis via 5′-silyl-2′-orthoester chemistry,” Methods 23, 206-217; Scaringe, S. and Caruthers, M. H. (1999) U.S. Pat. No. 5,889,136; Scaringe, S. and Caruthers, M. H. (1999) U.S. Pat. No. 6,008,400; Scaringe, S. (2000) U.S. Pat. No. 6,111,086; Scaringe, S. (2003) U.S. Pat. No. 6,590,093.

Transfection. HeLa cells were obtained from ATCC (Manassas, Va.). Cells were grown at 37° C. in a humidified atmosphere with 5% CO₂ in DMEM, 10% FBS, and L-Glutamine. All propagation media were further supplemented with penicillin (100 U/mL) and streptomycin (100 μg/mL). For transfection experiments, cells were seeded at 1.0-2.0×10⁴ cells/well in a 96 well plate, 24 hours before the experiment in antibiotic-free media. Cells were transfected with siRNA (100 nM) using Lipofectamine 2000 (0.25 μL/well, Invitrogen). For targeting of PPYLUC (U47295), cotransfections of plasmid and siRNA were performed using Lipofectamine 2000 at 0.5 μL/well in 293 cells at 2.5×10⁴ cells/well in a 96 well plate and harvested at 24 hours.

Gene Knockdown and Cell Viability Assay. Twenty-four to seventy-two hours post-transfection, the level of target knockdown was assessed using a branched DNA assay (Genospectra) specific for the target of interest. In all experiments, GAPDH (a housekeeping gene) was used as a reference. When GAPDH was the target gene, PPIB was used as a reference. All experiments were performed in triplicate and error bars represent standard deviation from the mean. For viability studies, 25 μl of AlamarBlue reagent (Trek Diagnostic Systems) was added to each well, and HEK293 cells were incubated 2 h at 37° C., 5% CO₂. Absorbance was then read at 570 nm using a 600 nm subtraction. The optical density (OD) is proportional to the number of viable cells in culture when the reading is in the linear range (0.6 to 0.9). Transfections resulting in an OD of ≧80% of control were considered nontoxic.

Microarray Experiments. For each sample, 1 μg of total RNA isolated from siRNA-treated cells was amplified and Cy5-labeled (Cy-5 CTP, Perkin Elmer) using Agilent's Low Input RNA Fluorescent Linear Amplification Kit and hybridized against Cy3 labelled material derived from lipid treated (control) samples. Hybridizations were performed using Agilent's Human 1A (V2) Oligo Microarrays (˜21,000 unique probes) according to the published protocol (750 ng each of Cy-3 and Cy-5 labelled sample loaded onto each array). Slides were washed using 6× and 0.06×SSPE (each with 0.025% N-lauroylsarcosine), dried using Agilent's nonaqueous drying and stabilization solution, and scanned on an Agilent Microarray Scanner (model G2505B). The raw image was processed using Feature Extraction software (v7.5.1). Further analysis was performed using Spotfire Decision Site 7.2 software and the Spotfire Functional Genomics Module. Outlier flagging was not used. Off-targets were identified as genes that were down-regulated by two-fold or more (log ratio of more than −0.3) by a given siRNA in at least one experiment, but were not modulated by other functionally equivalent siRNA targeting the same gene.

Computational Analysis. The Smith-Waterman local algorithm was implemented in C# and augmented to extend alignments along the entire length of the shorter aligned sequence. The implementation also allowed the use of either uniform match rewards/mismatch costs or scoring matrices, and either linear or single affine gap costs.

The first stage of analysis used this implementation to align each strand of 12 siRNAs (including one non-rationally designed siRNA) against all GenBank mRNAs represented on the microarray chip. The 1000 highest percent identity alignments (on either strand) for each siRNA were archived. The archived alignments were analyzed to determine their identity distributions and discover alignments with experimentally off-targeted mRNAs, using the validated dataset of 347 off-targets, including all accession numbers that were sequence-specifically down-regulated by 2-fold or more in at least one biological replicate.

The parameter-testing studies defined twelve scoring matrixes designed to reward complementarity rather than identity. Each scoring matrix was combined with at least one linear gap penalty (designed to allow only one gap at a time) and one single affine gap penalty (designed to allow multiple-gap runs) of varying weights to generate the 30 parameter sets. The dataset of experimental off-targets was limited to include only those 180 that were sequence-specifically down-regulated by approximately 2-fold or more in two biological replicates for the 11 rationally designed siRNAs and had well-annotated coding sequences. A control set was chosen at random from those mRNAs that were not significantly down-regulated by any of the test siRNAs, and assigned to the siRNAs in equal numbers as in the off-target set. For each parameter set, the S-W implementation was used to align each strand of the siRNAs with their off-targets' reversed mRNA (due to the complementary nature of the scoring matrices) and the best 20 alignments were archived; the process was repeated for the control set. Analysis identified the highest percent identity archived alignment for each siRNA/mRNA pair (including both strands) and generated histograms of these highest identity distributions for each dataset under each parameter set. Since all distributions except those for sets 29 and 30 were approximately normal, each off-target/control distribution pair except these two was subjected to a two-tailed T-test to determine whether their means were significantly different. The remaining two were subjected to a chi-squared test for independence. The results of all tests were adjusted using the Bonferroni correction to account for multiple comparisons. The analysis was also conducted for each strand individually.

The seed analysis was performed using a stringent subset of the experimentally validated off-targets including only those 84 with well-annotated UTRs that were sequence-specifically down-regulated by at least 2-fold in two biological replicates for 8 siRNAs measured in a single experiment; the control set was correspondingly narrowed. The analysis counted occurrences of exact substrings (identical to positions 13-18 inclusive, hexamer, and 12-18 inclusive, heptamer) of the siRNA sense strand to the 5′ UTR, ORF, and 3′ UTRs of each off-target and control.

### Example 1

### The Relevance of Overall Complementarity, Seeds, and 3′ UTRs

A database of experimentally validated off-targeted genes was generated from the expression signatures of HeLa cells transfected with one of twelve different siRNAs (100 nM) targeting three different genes, PPIB, MAP2K1, and GAPDH. Eleven rationally designed siRNA having a strong antisense (AS) strand bias toward RISC entry and one non-rationally designed siRNA were transfected into cells. Rationally designed siRNA were selected according to the methods disclosed in U.S. Patent Publication No. 2005/0255487 A1.

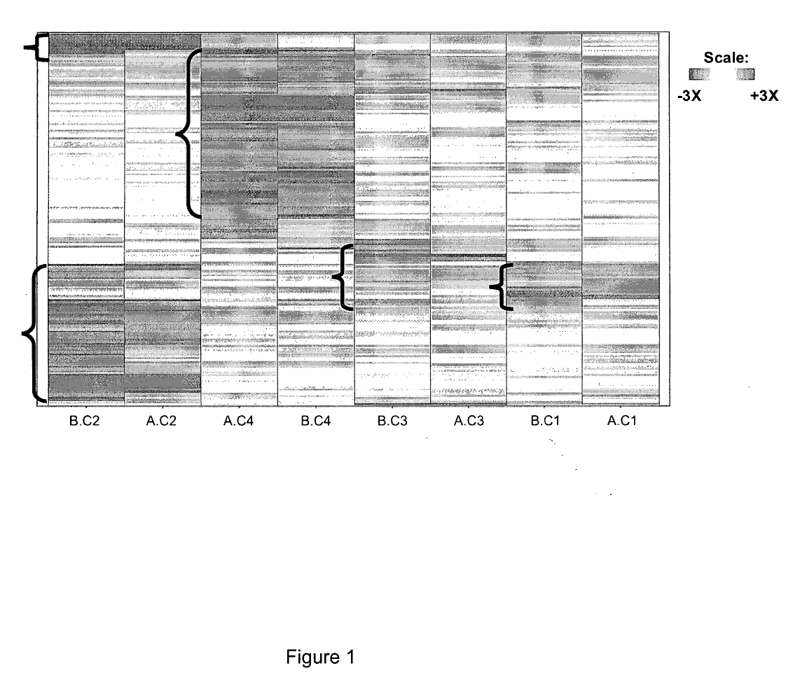

Genes that were down-regulated by two-fold or more (i.e. expression of 50% or less as compared to controls) by a given siRNA in one or more biological replicates, but were not modulated by other functionally equivalent siRNA targeting the same gene were designated as off-targets. Expression signatures of cells transfected with the 12 siRNAs identified 347 off-targeted genes. The expression signatures are shown in FIG. 1, which is a typical heatmap of HeLa cells transfected with four different PPIB-targeting siRNAs (C1, C2, C3, and C4). “A” and “B” represent biological replicates for transfection of each siRNA. Brackets highlight the clusters of sequence-specific off-targets of each siRNA.

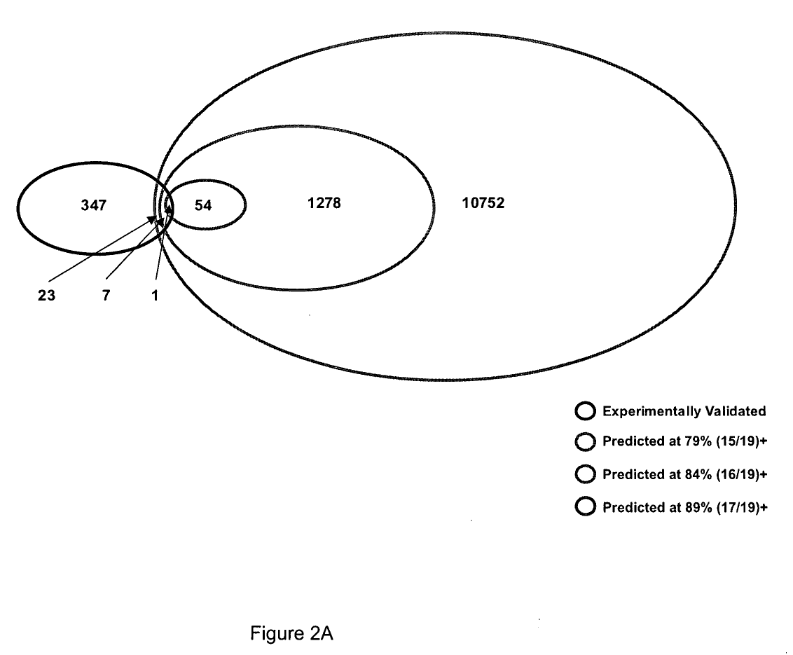

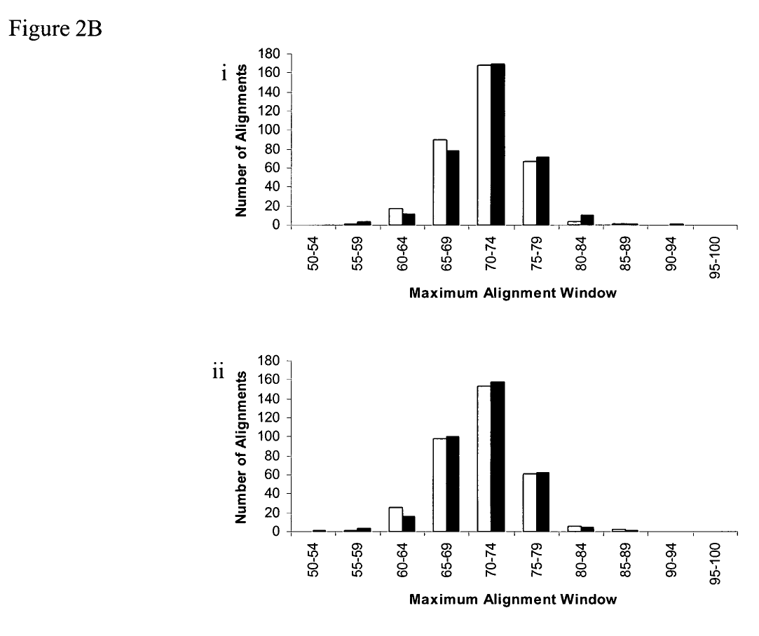

Tables IA-IC provide the siRNA sequence, intended target, list of validated off-targets and subsets of sequences that were used in each analysis. Table IA identifies the sequences used. Table IB provides data for the experimental results. Table IC provides the results for use in the sw1, sw2 and the seed analyses. “sw1” identifies the group of validated off-targets that were used to generate FIG. 2A. “sw2” identifies the group of validated off-targets that were used in the analysis of customized S-W parameter sets. The term “seed” identifies the group of validated off-targets that were used in the hexamer/heptamer seed analysis. Tables IA-IC below identify that the number of off-targets ranged from 5-73 genes per siRNA and the degree of down-regulation of this collection varied between approximately 2 and 5 fold.

Using the Smith Waterman alignment algorithm, the sense and antisense strands for each siRNA were aligned against the more than 20,000 genes represented on Agilent's Human 1A (V2) Oligo Microarray. Gene Sequences that exhibited ≧, 79% identity with either the sense or antisense strands were designated as in silico predicted off-targets. Commonly used reward/penalty parameters (a match reward=2, a mismatch penalty=−2, and a linear gap penalty=−3) were employed and a maximum cutoff of 1000 alignments per siRNA was arbitrarily imposed. (Although multiple alignments between a given siRNA and mRNA were recorded, analyses were done using only the best alignment between each pair). Surprisingly, the number of in silico predicted off-targets typically exceeded the number identified by microarray analysis by 1-2 orders of magnitude, regardless of whether alignments of one or both strands were included in the analysis. Thus, comparison of the validated off-target dataset with in silico predicted off-targets showed that identity cutoffs failed to accurately predict off-targeted genes.

Table II demonstrates the discrepancy between the number of validated off-targets for each siRNA and the predicted number of targets using different identity cutoffs. Predicted numbers are based on identity matches between the sense and antisense strand of the siRNA against the GenBank genes represented on Agilent's Human 1A (V2) Oligo Microarray. Table II below demonstrates a false positive rate of over 99% at the 79% identity cutoff. This number of predicted off-targets represented more than one third of the number of mRNAs in the human genome. Moreover, only 23 of the 347 experimentally validated off-targets were identified by in silico methods using this cutoff, which represents a false negative rate of approximately 93%. Higher cutoffs (>84% and >89%) produced similarly poor overlap between experimental and in silico target predictions (7 and 1 commonly identified targets using the 84%, and 89% identity filter, respectively), as well as gross mis-estimations of the number of off-targets (1278 and 54, respectively). Based on these observations, it was concluded that overall sequence identity was a poor predictor of the number and identity of off-targeted genes.

FIG. 2A is a Venn diagram that shows overlap between 347 experimentally identified off-targets and in silico off-targets predicted by the Smith-Waterman alignment algorithm. Left most set=347 experimentally validated off-targets for 12 separate siRNA. Outer, middle and inner gray right sets represent the number of off-targets predicted by S-W using ≧79% (e.g. 15/19 or better, 10752 off-targets), ≧84% (e.g. 16/19 or better, 1278 off-targets) and ≧89% (e.g. 17/19 or better, 54 off-targets) identity filters, respectively. The associated numbers (23, 7, and 1) represent the number of genes that are common between the experimental and predicted groups at each of the identity filter levels (≧79%, ≧84%, and ≧89%, respectively). The lack of relevance of overall identity in determining off-targets is demonstrated in FIG. 2B. The sense (top) and antisense (bottom) sequences of each siRNA were aligned separately to the sequences of their corresponding 347 experimentally validated off-targets and a comparable number of control untargeted genes to identify the alignments with the maximum percent identity. The number of alignments in each identity window were then plotted for the off-targeted (black) and untargeted (white) populations.

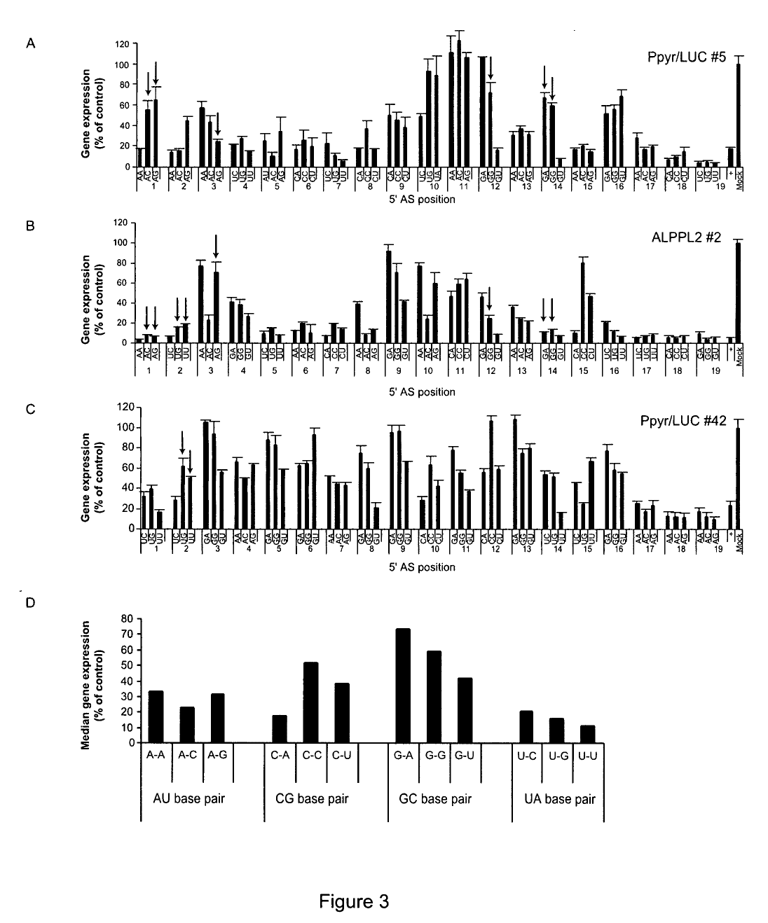

The inventors recognized that alignments are particularly sensitive to the weighting of matches, mismatches, and gaps. With the long term goal of creating a customized S-W parameter set that can distinguish between off-targeted and untargeted populations, individual siRNAs targeting human cyclophilin B (PPIB), firefly luciferase (PPYLUC), and secreted alkaline phosphatase (SEAP) were synthesized in their native state or with one of three base pair mismatches at each of the 19 positions of the duplex (48 variants per siRNA). Subsequently, a systematic single mismatch analysis of siRNA functionality was performed by transfecting each siRNA into HeLa cells and measuring the relative level of target silencing. The results of these experiments are presented in FIG. 3A-C and demonstrate several points.

First, Ppyr/LUC #5 and ALPPL2#2 studies clearly show that the central region of the duplex (positions 9-12) is particularly sensitive to mismatches. In contrast, duplexes with mismatches at positions 18 and 19 exhibit consistent silencing, suggesting that the strength of base pairing in this region is less critical. Outside of positions 9-12 and 18-19, the inventors observed that identical mismatches at any position could have widely disparate impacts on siRNA performance. Thus, for instance, while an A-G mismatch at position 3 of the Ppyr/LUC #5 has little impact on overall duplex functionality, the same mismatch at the same position in the ALPPL2#2 targeting siRNA dramatically alters silencing efficiency.

Second, G-A and G-G mismatches at position 14 of the ALPPL2 #2 siRNA have little or no effect on functionality, but identical mismatches at the same position in the Ppyr/LUC #5 siRNA result in a loss of activity. These findings suggest that with the exceptions of positions 18 and 19 (which appear to be insensitive to base pair mismatches) the complete sequence plays a role in determining the impact of mismatches, thus preventing the development of clear position-dependent mismatch criteria. Nonetheless, analysis of all mismatches in a position independent manner identifies a decided bias (FIG. 3D). In general, when mismatches are incorporated at U-A base pairs (e.g. U-C, U-G, or U-U) little change in functionality is observed. In contrast, when G-C base pairs are altered the overall effect on siRNA silencing is dramatic, with the effects of G-A being greater than those of G-G, which are in turn greater than those of G-U.

FIGS. 3A-3D demonstrate systematic single base pair-mismatch analysis of siRNA functionality. (A-C) Effects of single base pair mismatch in siRNAs targeting Ppyr\LUC #5(A), ALPPL2 #2 (B) and Ppyr\LUC #42 (C). Native forms of all three siRNAs induce >90% gene knockdown. Position 1 refers to the 5′-most position of the antisense strand. The top base represents the antisense mutation, and the bottom base represents the mismatched target site nucleotide. ‘Mock’, lipid-treated cells; ‘+’, native duplex. Arrows point to examples of positions that have equivalent bases with at least one other siRNA in the test group and show differences in functionality when particular base substitutions are made. Experiments were performed in triplicate. Error bars, s.d. from the mean. (D) Bar graph of overall impact of mismatch identity on siRNA function.

These observed biases were incorporated into 30 additional S-W parameter sets to test whether changes in the rewards/costs associated with matches and mismatches could improve the ability to predict off-targeted genes by overall alignment identity. Table III below describes the thirty custom S-W scoring parameters sets tested.

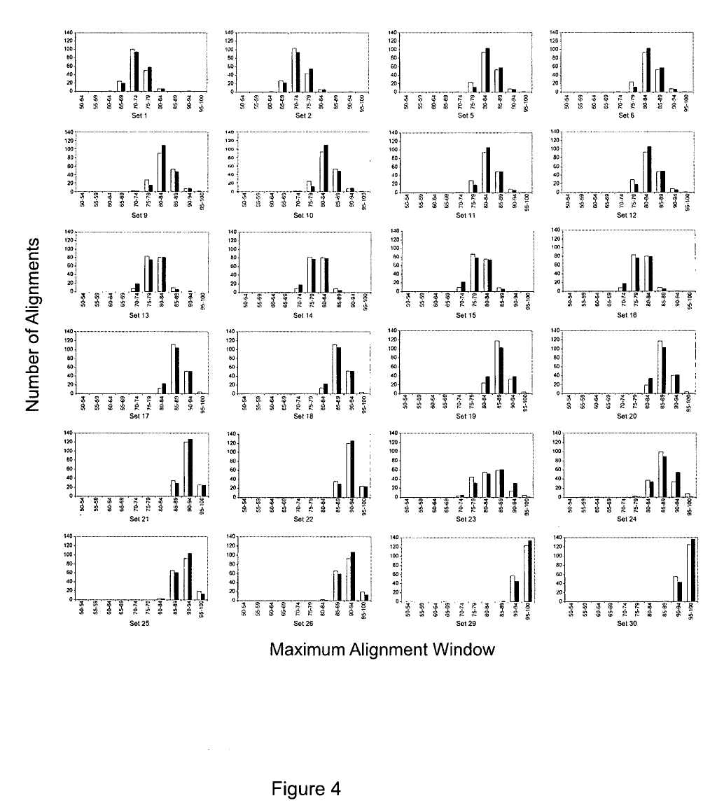

As it is unclear how gaps are tolerated by RNAi, several different gap penalties (both linear and affine) were included in the scoring matrices. Two populations of siRNA/mRNA pairs (180 representing experimentally validated off-target interactions and 180 having no discernable off-target interactions) were analyzed with each of the 30 unique scoring schemes. Analysis of off-targeted and untargeted populations using each of the modified parameter sets failed to distinguish between the two datasets regardless of whether alignments for one or both strands were included. The finding that the distributions of maximum identity in the best alignment for each parameter set for off-targeted and untargeted populations are statistically indistinguishable (p>0.05 after application of Bonferroni correction for multiple comparisons, FIG. 4) supports the previous conclusion that overall sequence identity is a poor predictor of off-targeted genes. Instead, the mechanism by which on-target and off-target gene regulation occurs may be mediated by other sets of factors and/or mechanisms.

FIG. 4 shows twenty-four of the thirty different parameter sets (Table III) that were tested to identify any that accurately distinguish off-targeted from untargeted genes. The sense and antisense sequences of each siRNA were aligned to the sequences (5′ UTR-ORF-3′ UTR) of their corresponding experimental off-targets (180 validated off-target sequences) and a comparable number of control untargeted genes to identify the maximum identity alignment according to each parameter set. The number of alignments (Y-axis) in each identity window (X-axis) were then plotted for the off-targeted (black) and untargeted (white) populations. (5′ UTR refers to the 5′ untranslated region. ORF refers to the open reading frame. 3′ UTR refers to the 3′ untranslated region.)

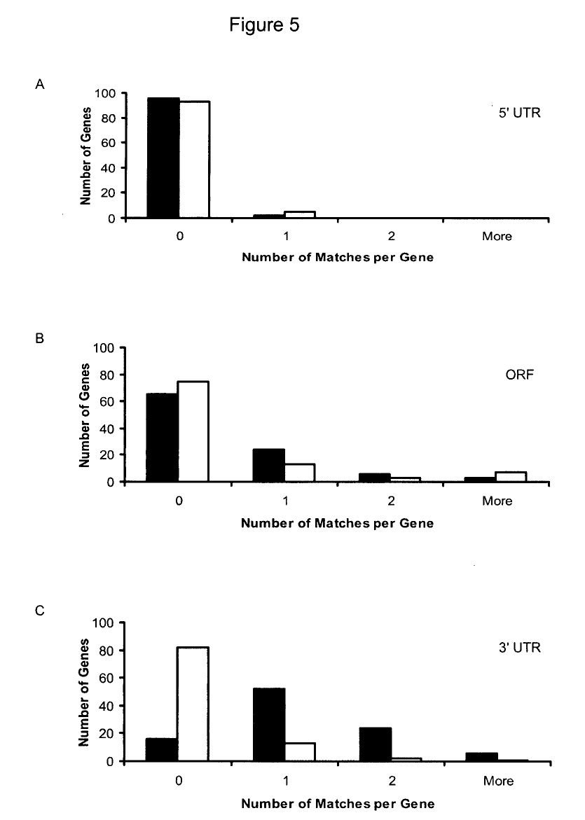

Recent studies on microRNA (miRNA) mediated gene modulation have shown that complementary base pairing between the seed sequence and sequences in the 3′ UTR of mRNA is associated with miRNA-mediated gene knockdown. (Lim et al., Microarray analysis shows that some microRNAs downregulate large numbers of target mRNAs, Nature 433, 769-73 (2005)). As siRNAs and miRNAs are believed to share some portion of the RNAi machinery, the inventors investigated whether complementarity between the seed sequence of the siRNA and any region of the transcript was associated with off-targeting. To accomplish this, the 5′ UTR, ORF, and 3′ UTR of 84 experimentally determined off-target genes were scanned for exact complementary matches to the antisense seed sequence (hexamer, positions 2-7, and heptamer, positions 2-8) of their respective siRNA. This dataset of siRNAs and their off-targeted genes was then compared to a control group (84 siRNA/mRNAs that shared no off-target interactions) to determine whether seed matches in any of the three regions correlated with off-targeting. For 5′ UTR and ORF sequences, the frequency at which one or more hexamer seed matches were present in the experimental and control groups was statistically indistinguishable (at the p>0.05 level using the chi squared test for independence, frequencies were 2.3% and 5.9% for the 5″ UTR, 30.9% and 23.8% for ORF sequences, respectively). In contrast, the incidence at which one or more hexamer matches were found in the 3′ UTR of off-targets was nearly 5-fold higher than that observed in the untargeted populations (84.5% in the experimental group, 17.8% in the control group; significant with p<0.001, FIG. 5). FIGS. 5A-5C show a search for complementarity between the siRNA antisense seed sequence (positions 2-7) and 5A, 5′ UTRs; 5B, ORFs; and 5C, 3′ UTRs of off-targeted (84 genes, black bars) and untargeted (84 genes, white bars) genes was performed. A strong association exists between exact hexamer matches and sequences in the 3′ UTR. Histograms generated for heptamer (2-8) seed matches also show correlation with 3′ UTR of off-targets (data not shown).

Furthermore, the positive predictive value (defined as [true positives]/[true positives+false positives]) of the association between 3′ UTR hexamer seed matches and off-targeted genes increased when multiple matches were required (for two or more 3′ UTR matches: off-targeted genes=29.76%, untargeted genes=3.57%) as shown in Table IV below, for sensitivity, specificity, and positive predictive power of siRNA hexamer and heptamer seed matches.

When four 3′ UTR hexamer seed matches are present, no false positives were detected in this limited sample. As seed matches provide an enhancement over the predictive abilities of blastn and S-W homology based searches, a search tool has been developed to enable identification of all possible human off-targets for any given siRNA based on 3′ UTR hexamer seed matches. The 3′ UTR hexamer identification tool takes the 19 base pair siRNA sense sequence, identifies the corresponding hexamer of the target site, and displays the identity of all genes carrying at least one perfect hexamer seed match in the 3′ UTR. A second column may display a smaller subset of genes that have two or more perfect 3′ UTR seed matches.

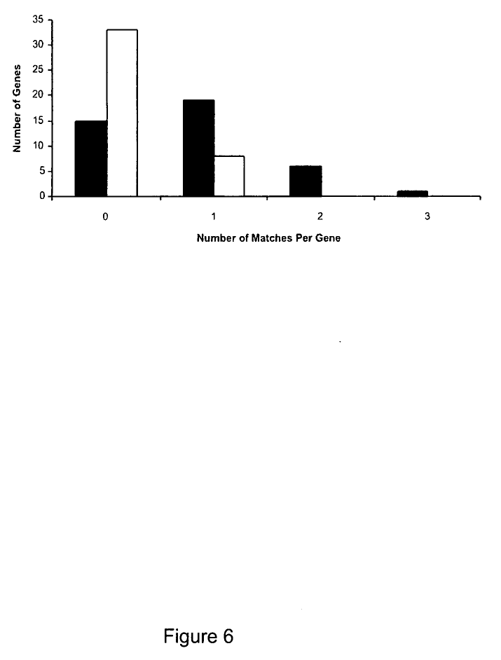

The frequency at which heptamer seed matches were observed in the 5′ UTR, ORF, and 3′ UTR of experimental and control groups was similar to those documented for hexamers (heptamer frequency in experimental and control groups: 5′ UTR: 0% and 1.2%; ORF: 16.6% and 9.5%; 3′ UTR: 69.1% and 8.3%) suggesting that the relevant seed sequence may consist of 7 nucleotides (positions 2-8), and the method of the present invention may be applied by focusing on either size region. As was observed with hexamer seed matches, increases in the numbers of 3′ UTR heptamer seed matches were associated with improvements in the specificity of the association. The observed associations remain after 3′ UTR length is controlled for by examining paired off-targeted and non-targeted control 3′ UTRs with lengths equal to within thirty bases (FIG. 6), thus suggesting that 3′ UTR-siRNA seed matches are an important parameter of off-targeting.

FIG. 6 demonstrates that seed sequence association with off-targeting is not due to 3′ UTR length. A search for complementarity between the siRNA antisense seed sequence (positions 2-7) and 3′ UTRs of off-targeted (41 genes, black bars) and untargeted (41 genes, white bars) genes with comparable 3′ UTR lengths was performed. The same association between exact hexamer matches and sequences in the 3′ UTR seen earlier is observed.

The work presented here demonstrates that with the exception of instances of near-perfect complementarity, the level of overall complementarity between an siRNA and any given mRNA is not associated with off-target identity. Both S-W and BLAST sequence alignment algorithms grossly overestimate the number of off-targeted genes when common thresholds are employed, suggesting that siRNA designed algorithms employing these methods may be discarding significant numbers of functional siRNAs due to unfounded specificity concerns. Moreover, the overlap between predicted and validated off-targets is minimal (0.2 to 5%) when identity thresholds ranging between >79% and >89% are employed. In addition, custom S-W parameters informed by base pair mismatch studies fail to produce alignments that distinguish between off-targeted and untargeted populations. These findings reveal that current protocols used to minimize off-target effects (e.g. BLAST and S-W) have little merit aside from eliminating the most obvious off-targets (i.e. sequences that have identical or near-identical target sites).

### Example 2

### Seed Frequencies in Human 3′ UTRs

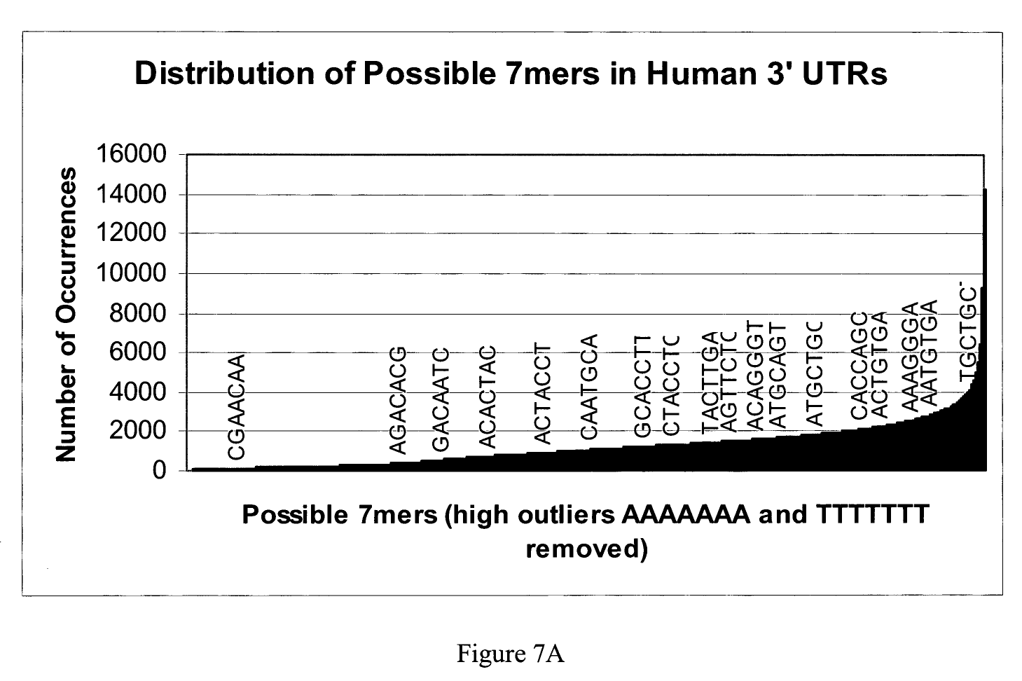

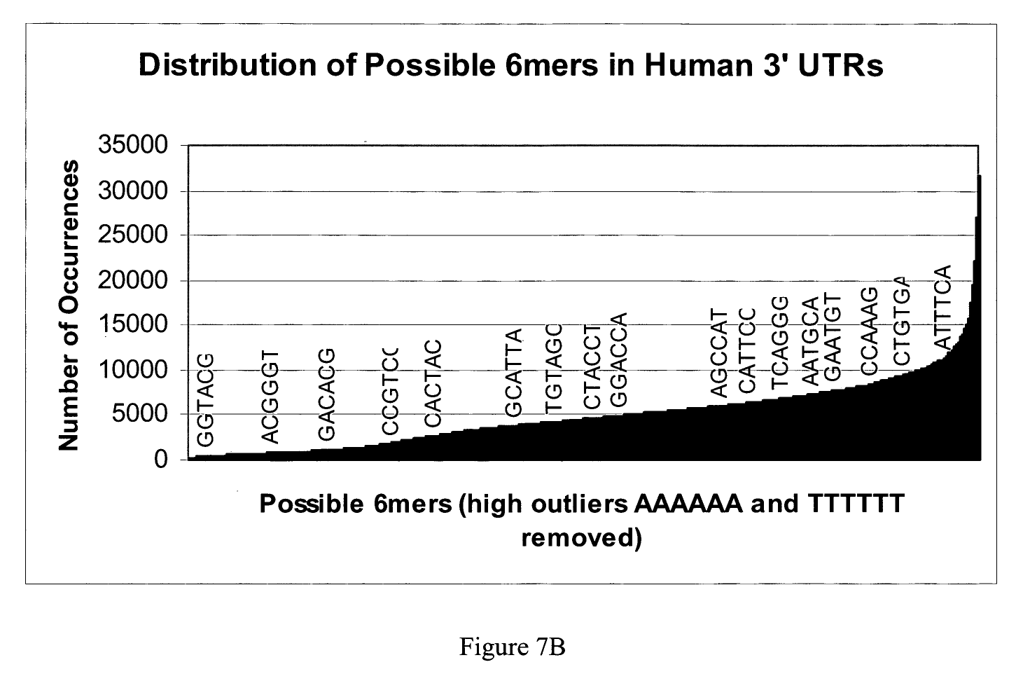

The sequences of human NM 3′ UTRs for RefSeq Version 17 were down loaded from NCBI (http://www.ncbi.nlm.nih.gov/). Subsequently, a comparison was made between these sequences and all 6 and 7 nt seeds (Lewis, B. P., C. B. Burge and D. P. Bartel. (2005) “Conserved seed pairing, often flanked by adenosines, indicates that thousands of human genes are microRNA targets,” Cell 120(1):15-20) to determine the frequency at which each possible hexamer/heptamer seed obtain was observed. The results, presented in FIG. 7, shows that the frequency of all seeds (hexamers or heptamers) is not equivalent.

### Example 3—Prophetic Example

### Application of Methods of the Present Invention

1. Identify Target Gene: The NCBI Entrez Gene database may be used to select a target gene and the corresponding sequence of record. Although it is possible to target individual transcripts or custom sequences, these gene records provide valuable information about known transcript variants. Whenever possible, one should use a gene's RefSeq mRNA variant rather than other related mRNA sequences, since the former have a greater likelihood to be complete and have well-annotated UTRs. In the course of this process, one must decide whether the designed siRNAs will target all known variants of the gene or only a specific subset, as well as which regions of the transcript(s) (5′ UTR, ORF, and/or 3′ UTR) may be targeted. In general, it is preferable to target the ORF; if suitable siRNAs cannot be designed for this region, the 3′ UTR may be included since the fraction of functional siRNAs in this region is similar to that for ORFs.
2. Build Candidate siRNA List: Based on the selected gene and the specified transcript variants to target, identify the regions that are common or unique to the specified variant(s) to define the target sequence space. Subsequently, generate all 21-base sequences within the selected region, discarding any that overlap with known SNPs or other polymorphisms that are annotated in any transcript's record. The remaining list represents the sense sequences of potential siRNA candidates for this gene; the final 19 bases (i.e. 3′ most 19 bases on the sense strand, which are opposite positions 1-19 of the antisense region) of each sense sequence, which participate in the siRNA duplex, are used in all subsequent steps. Reference is made to the sense strand because most publicly available databases contain sense strand information. However, unless otherwise specified reference to the sense strand includes methods and systems that work on principles of reverse complementarity and use data and information that has been input based on the antisense sequences.
3. Filter Candidates: Remove candidates with known functionality or specificity issues. These include duplexes containing (1) noncannonical bases; (2) more than 6 Gs and/or Cs in a row; (3) more than 4 of any single base in a row; (4) internal complementary stretches more than 3 bases long; (5) GC content less than 30%; (6) GC content greater than 64%; (7) toxic motifs such as GTCCTTCAA (Hornung, V., et al., Sequence-specific potent induction of IFN-alpha by short interfering RNA in plasmacytoid dendritic cells through TLR7. Nat. Med., 2005. 11(3): p. 263-270); or (8) seed complements found in miRNAs occurring across human, mouse, and rat.
4. Score Candidates: For each remaining candidate, calculate its functionality score based on thermodynamics and its base composition at each position. A wide selection of such scoring algorithms derived by a variety of means such as direct examination, decision trees, support vector machines, and neural networks are available. Higher scores indicate siRNAs with a greater chance of functionality.
5. Crop Candidate List: Sort the candidates in descending order of score and select the top 100; because sequence alignment is time-consuming, only these high scorers should be analyzed by blastn. This number may need to be increased in the case of hard-to-target genes. Note: Smith-Waterman can be substituted for blastn, with virtually the same outcome.
6. BLAST Candidates: Identify transcripts that may be unintentionally targeted for cleavage by the candidate siRNAs by running NCBI's blastn against a database such as RefSeq's mRNA entries. Because default blastn settings are inappropriate for very short sequences, the word size should be reduced to its minimum of 7 and the expect threshold should be increased to 1000. One should also consider reducing the default gap open and mismatch penalties to ensure that short, inexact matches, including those with small bulges, are correctly detected. Both the sense and antisense sequences can cause off-target cleavage, so a candidate with BLAST results for either strand indicating fewer than two mismatches with an unintended target should be considered undesirable.
7. Pick siRNAs: Examine the siRNAs analyzed by blastn and select at least four that balance high scores with short BLAST matches. Because siRNAs can also produce off-targets by translational repression, it is advisable to ensure that these final picks have a low frequency of seed matches to 3′ UTRs in the genome being targeted; for human and mouse, frequencies below 2000 are considered low. Multiple siRNAs should be picked in order to allow pooling (which can further reduce off-target effects) or independent confirmation of the phenotype produced by siRNA delivery.
8. Synthesize siRNAs: The picked siRNAs can be synthesized with a variety of chemical modifications to combat further possible off-target effects and enhance stability.

### Example 4

### Analyses of 3′ UTRs

When the 4096 possible hexamer seeds are binned by the number of human NM 3′ UTRs in which they appear, the resulting histogram shows a distinct bimodal distribution. The sharp secondary peak at the left of the histogram represents a distinct population of low-frequency seeds. (As shown in FIG. 8A, it appears that this low frequency is due to the ubiquitous presence of the CG dinucleotide in these seeds, as the CG dinucleotide is rare in mammals.)

The low frequency threshold of 2000 3′ UTRs was arrived at by determining the uppermost frequency limit of this rare-seed peak. In other animals (notably rat, in which the number of available NM RefSeq 3′ UTRs is only about ⅓ of that available for human) the 2000 threshold would not apply, but the bimodal distribution is still evident in FIG. 8B.

Thus, the threshold used for a particular organism (or for the human organism when designing against a later—and therefore larger—RefSeq database) should preferably be redetermined by plotting the above sort of histogram and selecting the upper limit of the rare seed peak. If this is not possible, then a percentage threshold may be applied (although it is not proven that the percentage of seeds in the low frequency peak is completely comparable between organisms); 2000 3′ UTRs represent approximately 8.5% of the currently known human transcriptome, so a reasonable percentage-based threshold would be to designate as low-frequency any seed that occurs in 8.5% or less of known transcripts for the genome in question. However, because the number of mRNAs for a given species and variability among the 3′ UTRs for those species, a cut off between 5% and 15% would generally be appropriate.

### Tables

TABLE IA

IDENTIFICATION OF SEQUENCES

| | | | |
|---|---|---|---|
| | | | target |
| siRNA id | siRNA Sense Seq | (SEQ ID NO.) | accession |

| | | | |
|---|---|---|---|
| C1 | GAAAGAGCAUCUACGGUGA | 1 | NM_000942 |
| C14 | GGCCUUAGCUACAGGAGAG | 2 | NM_000942 |
| C2 | GAAAGGAUUUGGCUACAAA | 3 | NM_000942 |
| C3 | ACAGCAAAUUCCAUCGUGU | 4 | NM_000942 |
| C4 | GGAAAGACUGUUCCAAAAA | 5 | NM_000942 |
| C52 | CAGGGCGGAGACUUCACCA | 6 | NM_000942 |
| G4 | UGGUUUACAUGUUCCAAUA | 7 | NM_002046 |
| G41 | GUAUGACAACAGCCUCAAG | 8 | NM_002046 |
| M1 | GCACAUGGAUGGAGGUUCU | 9 | NM_002755 |
| M2 | GCAGAGAGAGCAGAUUUGA | 10 | NM_002755 |
| M3 | GAGGUUCUCUGGAUCAAGU | 11 | NM_002755 |
| M4 | GAGCAGAUUUGAAGCAACU | 12 | NM_002755 |

TABLE IB
EXPERIMENTAL RESULTS

| | target | | | | |
|---|---|---|---|---|---|
| siRNA id | accession | new accession | GeneName | experiment 1 | experiment 2 |
| C1 | NM_000942 | NM_014686 | I_962629 | −0.33 | −0.12 |
| | | AL080111 | NEK7 | −0.33 | −0.31 |
| | | NM_012238 | SIRT1 | 0.11 | −0.33 |
| | | NM_005000 | NDUFA5 | −0.37 | −0.41 |
| | | NM_006868 | RAB31 | −0.30 | −0.35 |
| | | BC002461 | BNIP2 | −0.16 | −0.31 |
| | | NM_002628 | PFN2 | −0.38 | −0.24 |
| | | NM_002296 | LBR | −0.43 | −0.41 |
| | | NM_006805 | HNRPA0 | −0.26 | −0.31 |
| | | NM_006579 | EBP | −0.31 | −0.36 |
| | | ENST00000199168 | B4GALT1 | −0.41 | −0.41 |
| | | NM_024420 | PLA2G4A | −0.43 | −0.38 |
| | | NM_001497 | NM_001497.2 | −0.36 | −0.33 |
| | | NM_003574 | VAPA | −0.28 | −0.40 |
| | | NM_006216 | SERPINE2 | −0.35 | −0.37 |
| | | NM_013233 | STK39 | −0.42 | −0.46 |
| | | AK000313 | FLJ20306 | −0.31 | 0.02 |
| | | NM_022725 | FANCF | −0.34 | −0.32 |
| | | NM_022780 | FLJ13910 | −0.34 | −0.36 |
| | | NM_032012 | C9orf5 | −0.41 | −0.42 |
| | | NM_152780 | NM_152780.1 | −0.31 | −0.24 |
| | | NM_153812 | NM_153812.1 | −0.10 | −0.30 |
| | | NM_002078 | GOLGA4 | −0.35 | −0.36 |
| | | NM_003089 | SNRP70 | −0.32 | −0.14 |
| | | NM_004396 | DDX5 | −0.26 | −0.37 |
| | | NM_001698 | AUH | −0.33 | −0.31 |
| | | NM_004568 | SERPINB6 | −0.37 | −0.16 |
| C14 | NM_000942 | NM_003677 | DENR | −0.315 | −0.323 |
| | | NM_018371 | ChGn | −0.338 | −0.247 |
| | | NM_006587 | PRSC | −0.306 | −0.239 |
| | | NM_016097 | HSPC039 | −0.357 | −0.415 |
| | | NM_015224 | RAP140 | −0.202 | −0.325 |
| | | NM_020726 | NLN | −0.188 | −0.309 |
| | | NM_004436 | ENSA | −0.29 | −0.252 |
| | | NM_021158 | C20orf97 | −0.504 | −0.601 |
| | | AK056178 | I_961477 | −0.162 | −0.257 |
| | | NM_015134 | I_1109594 | −0.161 | −0.325 |
| | | NM_016059 | PPIL1 | −0.276 | −0.337 |
| | | NM_006600 | NUDC | −0.52 | −0.553 |
| | | ENST00000307767 | I_958489 | −0.325 | −0.378 |
| | | NM_004550 | NDUFS2 | −0.341 | −0.345 |
| | | NM_024329 | MGC4342 | −0.274 | −0.328 |
| | | NM_017845 | FLJ20502 | −0.358 | −0.406 |
| | | BC039726 | GTF2H3 | −0.317 | −0.408 |
| | | NM_001554 | CYR61 | −0.355 | −0.309 |
| | | AK057783 | I_958429 | −0.267 | −0.388 |
| | | NM_007222 | ZHX1 | −0.361 | −0.245 |
| | | NM_199133 | I_958324 | −0.304 | −0.372 |
| | | Z24727 | I_960077 | −0.253 | −0.307 |
| | | NM_001765 | CD1C | −0.0637 | −0.392 |
| | | NM_005012 | ROR1 | −0.35 | −0.342 |
| | | NM_000092 | COL4A4 | −0.18 | −0.312 |
| | | NM_000356 | TCOF1 | −0.362 | −0.406 |
| | | NM_001516 | NM_001516.3 | −0.348 | −0.378 |
| | | NM_002816 | I_964302 | −0.296 | −0.333 |
| | | NM_002826 | QSCN6 | −0.466 | −0.543 |
| | | NM_002840 | I_931679 | −0.334 | −0.357 |
| | | NM_004287 | GOSR2 | −0.311 | −0.257 |
| | | NM_005414 | NM_005414.1 | −0.0676 | −0.327 |
| | | NM_015532 | GRINL1A | −0.443 | −0.425 |
| | | NM_015650 | MIP-T3 | −0.201 | −0.308 |
| | | NM_016341 | PLCE1 | −0.0259 | −0.364 |
| | | NM_181354 | OXR1 | −0.34 | −0.329 |
| | | NM_018979 | NM_018979.1 | −0.368 | −0.244 |
| | | NM_022121 | NM_022121.1 | −0.621 | −0.651 |
| | | NM_024699 | FLJ14007 | −0.303 | −0.167 |
| | | NM_032690 | MGC13198 | −0.272 | −0.325 |
| | | NM_134428 | RFX3 | −0.0828 | −0.309 |
| | | NM_152437 | NM_152437.1 | −0.349 | −0.389 |
| | | NM_001168 | BIRC5 | −0.307 | −0.303 |
| | | ENST00000269463 | MAPK4 | −0.253 | −0.358 |
| | | NM_005647 | TBL1X | −0.271 | −0.341 |
| | | NM_016441 | CRIM1 | −0.34 | −0.42 |
| C2 | NM_000942 | NM_014342 | MTCH2 | −0.30 | −0.25 |
| | | NM_014517 | UBP1 | −0.31 | −0.27 |
| | | BX538238 | RPLP1 | −0.18 | −0.36 |
| | | NM_001755 | CBFB | −0.30 | −0.35 |
| | | NM_004433 | ELF3 | −0.27 | −0.35 |
| | | NM_016131 | RAB10 | −0.45 | −0.55 |
| | | NM_024054 | MGC2821 | −0.31 | −0.33 |
| | | NM_145808 | V-1 | −0.31 | −0.34 |
| | | A_23_P60699 | I_1109406 | −0.70 | −0.64 |
| | | AL832848 | I_958969 | −0.32 | −0.32 |
| | | NM_032783 | FLJ14431 | −0.30 | −0.34 |
| | | NM_000117 | EMD | −0.03 | −0.31 |
| | | NM_001412 | EIF1A | −0.37 | −0.35 |
| | | NM_001933 | DLST | 0.15 | −0.32 |
| | | NM_012106 | BART1 | −0.49 | −0.50 |
| | | NM_014316 | CARHSP1 | 0.00 | −0.30 |
| | | NM_001710 | BF | −0.14 | −0.31 |
| | | NM_006457 | LIM | −0.20 | −0.31 |
| | | NM_006016 | CD164 | −0.42 | −0.33 |
| | | NM_145058 | MGC7036 | −0.29 | −0.33 |
| | | NM_018471 | HT010 | −0.35 | −0.26 |
| | | NM_003211 | TDG | −0.33 | −0.18 |
| | | NM_002901 | RCN1 | −0.51 | −0.56 |
| | | NM_014888 | FAM3C | −0.31 | −0.16 |
| | | NM_005629 | SLC6A8 | −0.20 | −0.32 |
| | | NM_001549 | IFIT4 | −0.20 | −0.42 |
| | | NM_013354 | CNOT7 | −0.41 | −0.37 |
| | | NM_013994 | DDR1 | −0.19 | −0.32 |
| | | AB020721 | FAM13A1 | −0.14 | −0.31 |
| | | NM_014891 | PDAP1 | −0.31 | −0.27 |
| | | NM_016090 | RBM7 | −0.21 | −0.32 |
| | | AK098212 | FLJ10359 | −0.30 | −0.35 |
| | | NM_022469 | NM_022469.1 | −0.21 | −0.31 |
| | | NM_002136 | HNRPA1 | −0.41 | −0.34 |
| | | NM_080655 | MGC17337 | −0.26 | −0.36 |
| | | NM_138358 | NM_138358.1 | −0.43 | −0.40 |
| | | BC021238 | NM_144975.1 | −0.07 | −0.40 |
| | | NM_173705 | MTCO2 | −0.21 | −0.36 |
| | | NM_173714 | MTND6 | −0.21 | −0.32 |
| | | NM_004318 | ASPH | −0.11 | −0.40 |
| | | NM_005079 | TPD52 | −0.60 | −0.50 |
| | | NM_021990 | GABRE | −0.16 | −0.35 |
| | | NM_002245 | KCNK1 | −0.27 | −0.38 |
| | | U79751 | BLZF1 | −0.29 | −0.38 |
| | | NM_002273 | KRT8 | −0.30 | −0.41 |
| C3 | NM_000942 | NM_005467 | NAALAD2 | −0.17 | −0.34 |
| | | NM_007219 | RNF24 | −0.04 | −0.31 |
| | | NM_005359 | MADH4 | −0.16 | −0.38 |
| | | NM_018464 | MDS029 | −0.27 | −0.30 |
| | | THC1978535 | SPC18 | −0.46 | −0.42 |
| | | BC035054 | I_1152453 | −0.12 | −0.36 |
| | | NM_014300 | NM_014300.1 | −0.39 | −0.44 |
| | | AB014585 | I_962909 | −0.19 | −0.34 |
| | | NM_017798 | C20orf21 | −0.29 | −0.35 |
| | | BC007917 | I_1110079 | −0.32 | 0.08 |
| | | NM_033503 | NM_033503.2 | −0.08 | −0.31 |
| | | NM_152898 | FERD3L | −0.43 | −0.35 |
| C4 | NM_000942 | NM_015927 | TGFB1I1 | −0.32 | −0.38 |
| | | NM_018492 | TOPK | −0.38 | −0.30 |
| | | NM_016639 | TNFRSF12A | −0.30 | −0.10 |
| | | NM_002815 | PSMD11 | −0.30 | −0.25 |
| | | NM_004386 | CSPG3 | −0.36 | −0.32 |
| | | NM_006464 | TGOLN2 | −0.26 | −0.35 |
| | | NM_001047 | SRD5A1 | −0.31 | −0.23 |
| | | NM_012428 | SDFR1 | −0.41 | −0.34 |
| | | BC033809 | SNX12 | −0.33 | −0.26 |
| | | NM_032026 | CDA11 | −0.32 | −0.07 |
| | | NM_016436 | C20orf104 | −0.33 | −0.36 |
| | | NM_022083 | C1orf24 | −0.17 | −0.33 |
| | | NM_018018 | SLC38A4 | −0.32 | −0.24 |
| | | A_23_P67028 | I_1151840 | −0.37 | −0.30 |
| | | BC013629 | PRKWNK1 | −0.32 | −0.23 |
| | | NM_013397 | I_966759 | −0.43 | −0.46 |
| | | NM_012091 | ADAT1 | −0.31 | −0.28 |
| | | NM_030980 | FLJ12671 | −0.34 | −0.24 |
| | | NM_020898 | KIAA1536 | −0.31 | −0.15 |
| | | THC1990950 | FLJ30663 | −0.22 | −0.32 |
| | | NM_006818 | AF1Q | −0.36 | −0.31 |
| | | NM_012388 | PLDN | −0.37 | −0.15 |
| | | NM_001753 | CAV1 | −0.31 | −0.37 |
| | | NM_178129 | I_1000556 | −0.30 | −0.21 |
| | | NM_020374 | C12orf4 | −0.43 | −0.35 |
| | | NM_003739 | AKR1C3 | −0.49 | −0.45 |
| | | NM_000691 | ALDH3A1 | −0.25 | −0.31 |
| | | NM_006835 | CCNI | −0.21 | −0.31 |
| | | NM_206858 | PPP1R2 | −0.52 | −0.39 |
| | | NM_022145 | FKSG14 | −0.24 | −0.37 |
| | | NM_000104 | CYP1B1 | −0.43 | −0.54 |
| | | NM_005168 | ARHE | −0.31 | −0.29 |
| | | A_23_P84016 | ARF4 | −0.47 | −0.44 |
| | | NM_002444 | MSN | −0.28 | −0.31 |
| | | NM_016302 | LOC51185 | −0.30 | −0.30 |
| | | BC025376 | I_950244 | −0.31 | −0.10 |
| | | NM_021258 | IL22RA1 | −0.17 | −0.30 |
| | | NM_003472 | DEK | −0.29 | −0.37 |
| | | NM_000088 | COL1A1 | −0.25 | −0.49 |
| | | NM_174887 | LOC90410 | −0.34 | −0.28 |
| | | NM_031954 | MSTP028 | −0.42 | −0.35 |
| | | NM_002061 | GCLM | −0.37 | −0.43 |
| | | NM_004788 | UBE4A | −0.30 | −0.23 |
| | | NM_001387 | DPYSL3 | −0.42 | −0.48 |
| | | NM_001086 | AADAC | −0.34 | −0.29 |
| | | NM_004470 | FKBP2 | −0.54 | −0.60 |
| | | NM_005231 | EMS1 | −0.36 | −0.20 |
| | | NM_000189 | HK2 | −0.25 | −0.34 |
| | | NM_001535 | HRMT1L1 | −0.34 | −0.20 |
| | | NM_001660 | NM_001660.2 | −0.43 | −0.43 |
| | | NM_001754 | RUNX1 | −0.23 | −0.32 |
| | | NM_002094 | GSPT1 | −0.31 | −0.17 |
| | | NM_003286 | NM_003286.2 | −0.37 | −0.07 |
| | | NM_016823 | I_1109823 | −0.34 | −0.11 |
| | | NM_006764 | IFRD2 | −0.50 | −0.47 |
| | | NM_012383 | OSTF1 | −0.21 | −0.32 |
| | | AK000796 | C14orf129 | −0.32 | −0.17 |
| | | NM_018132 | FLJ10545 | −0.40 | −0.31 |
| | | NM_018390 | I_964018 | −0.32 | −0.30 |
| | | NM_020314 | MGC16824 | −0.33 | −0.20 |
| | | NM_021156 | DJ971N18.2 | −0.33 | −0.31 |
| | | NM_022074 | FLJ22794 | −0.34 | −0.18 |
| | | NM_032132 | NM_032132.1 | −0.27 | −0.32 |
| | | NM_080546 | CDW92 | −0.41 | −0.38 |
| | | NM_080725 | C20orf139 | −0.38 | −0.31 |
| | | NM_080927 | ESDN | −0.29 | −0.32 |
| | | NM_152344 | NM_152344.1 | −0.33 | −0.27 |
| | | NM_152523 | FLJ40432 | −0.26 | −0.44 |
| | | NM_000408 | GPD2 | −0.37 | −0.41 |
| | | NM_003675 | PRPF18 | −0.40 | −0.33 |
| | | NM_001425 | EMP3 | −0.33 | −0.25 |
| | | NM_006825 | CKAP4 | −0.31 | −0.36 |
| | | NM_022360 | FAM12B | −0.35 | −0.08 |
| C52 | NM_000942 | AB011134 | KIAA0562 | −0.39 | −0.38 |
| | | NM_002705 | PPL | 0.18 | −0.31 |
| | | NM_002317 | LOX | 0.33 | −0.32 |
| | | NM_006594 | AP4B1 | −0.32 | −0.05 |
| | | NM_018004 | FLJ10134 | 0.18 | −0.49 |
| | | AL137442 | C20orf177 | −0.32 | −0.26 |
| | | NM_024071 | MGC2550 | −0.40 | −0.40 |
| | | NM_002925 | RGS10 | −0.28 | −0.30 |
| | | NM_006773 | DDX18 | −0.32 | −0.11 |
| | | NM_003370 | VASP | −0.32 | −0.33 |
| | | NM_052859 | RFT1 | −0.35 | −0.12 |
| | | NM_014344 | FJX1 | −0.31 | −0.16 |
| | | NM_006285 | TESK1 | −0.22 | −0.35 |
| | | NM_000303 | PMM2 | −0.40 | −0.43 |
| | | NM_000723 | CACNB1 | −0.31 | −0.05 |
| | | NM_003731 | I_962660 | −0.41 | −0.30 |
| | | NM_004042 | ARSF | −0.31 | −0.26 |
| | | NM_004354 | CCNG2 | 0.11 | −0.30 |
| | | NM_005417 | SRC | −0.37 | −0.25 |
| | | NM_012207 | HNRPH3 | −0.31 | −0.14 |
| | | NM_014298 | QPRT | −0.39 | −0.33 |
| | | NM_015947 | CGI-18 | −0.33 | −0.51 |
| | | NM_016479 | I_951081 | −0.52 | −0.56 |
| | | NM_017590 | RoXaN | −0.32 | −0.31 |
| | | NM_018685 | NM_018685.1 | −0.33 | −0.23 |
| | | NM_020188 | DC13 | −0.44 | −0.43 |
| | | NM_025147 | FLJ13448 | −0.33 | −0.15 |
| | | NM_025198 | LOC80298 | −0.30 | −0.08 |
| | | NM_032620 | GTPBG3 | −0.33 | −0.21 |
| | | NM_033502 | TReP-132 | −0.35 | −0.17 |
| | | NM_145110 | MAP2K3 | −0.35 | −0.30 |
| | | THC1943229 | I_1110140 | −0.30 | −0.27 |
| | | NM_173607 | C14orf24 | −0.31 | −0.31 |
| | | NM_000389 | CDKN1A | 0.02 | −0.30 |
| | | THC1961572 | NOG | 0.15 | −0.33 |
| | | NM_004380 | CREBBP | −0.40 | −0.19 |
| | | NM_002857 | PXF | −0.32 | −0.04 |
| G4 | NM_002046 | NM_198278 | I_1201835 | −0.419 | −0.43 |
| | | NM_015584 | DKFZP586F1524 | −0.264 | −0.31 |
| | | NM_002720 | PPP4C | −0.381 | −0.392 |
| | | AY359048 | I_1891255.FL1 | −0.278 | −0.381 |
| | | NM_005349 | I_957839 | −0.277 | −0.316 |
| | | D14041 | KBF2 | −0.236 | −0.326 |
| G41 | NM_002046 | NM_033520 | I_966130 | −0.208 | −0.382 |
| | | NM_006554 | MTX2 | −0.336 | −0.35 |
| | | NM_016441 | CRIM1 | −0.391 | −0.398 |
| | | NM_022163 | MRPL46 | −0.282 | −0.357 |
| | | NM_020381 | LOC57107 | −0.339 | −0.335 |
| | | NM_002109 | HARS | −0.38 | −0.401 |
| | | NM_013402 | FADS1 | −0.336 | −0.209 |
| | | NM_033515 | MacGAP | −0.284 | −0.397 |
| | | NM_004060 | CCNG1 | −0.293 | −0.469 |
| | | NM_004096 | EIF4EBP2 | −0.34 | −0.336 |
| | | NM_017946 | FKBP14 | −0.305 | −0.369 |
| | | NM_002524 | NRAS | −0.393 | −0.361 |
| | | NM_002834 | I_1000320 | −0.481 | −0.443 |
| | | A_23_P165819 | CALM2 | −0.321 | −0.453 |
| | | BC029424 | I_1204326 | −0.317 | −0.258 |
| | | D31887 | KIAA0062 | −0.292 | −0.348 |
| | | NM_001387 | DPYSL3 | −0.315 | −0.394 |
| | | NM_001921 | DCTD | −0.53 | −0.531 |
| | | NM_007096 | CLTA | −0.399 | −0.406 |
| | | NM_001349 | DARS | −0.379 | −0.376 |
| | | NM_001743 | NM_001743.3 | −0.505 | −0.458 |
| | | NM_001943 | DSG2 | −0.319 | −0.328 |
| | | NM_002721 | NM_002721.3 | −0.315 | −0.377 |
| | | NM_003501 | ACOX3 | −0.361 | −0.329 |
| | | NM_004261 | SEP15 | −0.3 | −0.346 |
| | | NM_006759 | UGP2 | −0.363 | −0.361 |
| | | NM_018046 | FLJ10283 | −0.378 | −0.334 |
| | | NM_018192 | MLAT4 | −0.35 | −0.35 |
| | | NM_032132 | NM_032132.1 | −0.256 | −0.331 |
| | | NM_052839 | PANX2 | −0.335 | −0.00303 |
| | | NM_002190 | I_957599 | −0.322 | −0.157 |
| | | ENST00000328742 | I_929270 | −0.348 | −0.387 |
| | | NM_002346 | LY6E | −0.443 | −0.421 |
| | | NM_002133 | HMOX1 | −0.486 | −0.401 |
| | | NM_001628 | AKR1B1 | −0.347 | −0.385 |
| | | NM_000138 | FBN1 | −0.294 | −0.311 |
| M1 | NM_002755 | NM_015055 | SWAP70 | −0.31 | −0.13 |
| | | NM_016047 | CGI−110 | −0.56 | −0.48 |
| | | NM_018250 | FLJ10871 | −0.50 | −0.30 |
| | | NM_138467 | I_1000003 | −0.35 | −0.36 |
| | | NM_017845 | FLJ20502 | −0.39 | −0.29 |
| | | NM_005567 | LGALS3BP | −0.33 | −0.33 |
| | | NM_006345 | C4orf1 | −0.36 | −0.25 |
| | | NM_001724 | BPGM | −0.33 | −0.14 |
| | | NM_021913 | AXL | −0.41 | −0.54 |
| | | NM_005895 | GOLGA3 | −0.32 | −0.23 |
| | | NM_005349 | I_957839 | −0.31 | −0.23 |
| | | NM_006711 | RNPS1 | −0.40 | −0.41 |
| | | NM_001087 | AAMP | −0.40 | −0.58 |
| | | NM_002185 | IL7R | −0.43 | −0.41 |
| | | NM_012347 | FBXO9 | −0.30 | −0.21 |
| | | NM_014033 | NM_014033.1 | −0.31 | −0.16 |
| | | NM_014889 | PITRM1 | −0.39 | −0.33 |
| | | NM_001981 | PRO1866 | −0.38 | −0.27 |
| | | NM_032122 | DTNBP1 | −0.42 | −0.40 |
| | | NM_005877 | I_1110043 | −0.33 | −0.45 |
| | | NM_153812 | NM_153812.1 | −0.33 | −0.22 |
| | | NM_004311 | ARL3 | −0.40 | −0.43 |
| | | NM_001379 | DNMT1 | −0.43 | −0.37 |
| | | NM_001494 | GDI2 | −0.35 | −0.29 |
| M2 | NM_002755 | NM_014908 | KIAA1094 | −0.34 | −0.35 |
| | | NM_020062 | SLC2A4RG | −0.49 | −0.36 |
| | | NM_018686 | CMAS | −0.34 | −0.25 |
| | | NM_021238 | TERA | −0.34 | −0.18 |
| | | NM_004965 | HMGN1 | −0.36 | −0.36 |
| | | NM_014374 | RIP60 | −0.41 | −0.40 |
| | | NM_014670 | BZW1 | −0.31 | −0.25 |
| | | NM_018429 | BDP1 | −0.39 | −0.29 |
| | | NM_020470 | YIF1P | −0.29 | −0.34 |
| | | NM_020820 | NM_020820.1 | −0.34 | −0.15 |
| | | NM_004731 | SLC16A7 | −0.31 | −0.22 |
| M3 | NM_002755 | NM_078470 | COX15 | −0.40 | −0.33 |
| | | NM_032574 | LOG84661 | −0.37 | −0.35 |
| | | NM_001948 | DUT | −0.30 | −0.20 |
| | | NM_002657 | PLAGL2 | −0.31 | −0.14 |
| | | NM_012249 | TC10 | −0.56 | −0.19 |
| | | NM_152344 | NM_152344.1 | −0.31 | −0.25 |
| M4 | NM_002755 | AB002370 | KIAA0372 | −0.33 | −0.23 |
| | | NM_004844 | SH3BP5 | −0.32 | −0.22 |
| | | NM_015455 | I_957034 | −0.38 | −0.35 |
| | | NM_016542 | MST4 | −0.31 | −0.27 |
| | | NM_001262 | CDKN2C | −0.33 | −0.29 |
| | | NM_198969 | AES | −0.31 | −0.23 |
| | | NM_012428 | SDFR1 | −0.33 | −0.39 |
| | | NM_013372 | I_1876431.FL1 | −0.39 | −0.41 |
| | | NM_013237 | PX19 | −0.36 | −0.37 |
| | | NM_014071 | NCOA6 | −0.39 | −0.29 |
| | | NM_014112 | TRPS1 | −0.34 | −0.29 |
| | | NM_022740 | I_1201825 | −0.41 | −0.32 |
| | | NM_138444 | LOC115207 | −0.40 | −0.41 |
| | | BC032468 | I_1000199 | −0.33 | −0.34 |
| | | NM_015134 | I_1109594 | −0.43 | −0.38 |
| | | NM_000691 | ALDH3A1 | −0.24 | −0.39 |
| | | NM_002902 | RCN2 | −0.50 | −0.42 |
| | | NM_022149 | MAGEF1 | −0.33 | −0.14 |
| | | NM_016619 | PLAC8 | −0.21 | −0.33 |
| | | NM_002960 | S100A3 | −0.41 | −0.33 |
| | | NM_031286 | SH3BGRL3 | −0.40 | −0.42 |
| | | NM_003472 | DEK | −0.43 | −0.34 |
| | | NM_032124 | DKFZP564D1378 | −0.33 | −0.37 |
| | | NM_014615 | KIAA0182 | −0.34 | −0.21 |
| | | NM_003200 | TCF3 | −0.42 | −0.35 |
| | | NM_004120 | GBP2 | −0.32 | −0.24 |
| | | NM_021137 | TNFAIP1 | −0.30 | −0.20 |
| | | NM_006756 | TCEA1 | −0.35 | −0.30 |
| | | NM_002224 | ITPR3 | −0.33 | −0.20 |
| | | NM_005120 | TNRC11 | −0.33 | −0.24 |
| | | NM_006628 | ARPP−19 | −0.37 | −0.40 |
| | | NM_012207 | HNRPH3 | −0.37 | −0.35 |
| | | NM_016516 | HCC8 | −0.32 | −0.18 |
| | | NM_025075 | FLJ23445 | −0.32 | −0.26 |
| | | NM_031427 | MGC12435 | −0.26 | −0.31 |
| | | NM_004176 | SREBF1 | −0.41 | −0.27 |
| | | THC1811009 | TMPO | −0.31 | −0.23 |
| | | NM_002522 | NPTX1 | −0.39 | −0.27 |
| | | NM_139045 | SMARCA2 | −0.38 | −0.35 |

TABLE IC
RESULTS FOR USE IN SW1, SW2 and SEED

| siRNA | | | | |
|---|---|---|---|---|
| id | new accession | used in sw1 | used in sw2 | used in seed |
| C1 | NM_014686 | TRUE | FALSE | FALSE |
| | AL080111 | FALSE | TRUE | FALSE |
| | NM_012238 | TRUE | FALSE | FALSE |
| | NM_005000 | TRUE | TRUE | TRUE |
| | NM_006868 | FALSE | TRUE | FALSE |
| | BC002461 | TRUE | FALSE | FALSE |
| | NM_002628 | FALSE | TRUE | FALSE |
| | NM_002296 | FALSE | TRUE | FALSE |
| | NM_006805 | TRUE | TRUE | FALSE |
| | NM_006579 | FALSE | TRUE | FALSE |
| | ENST00000199168 | FALSE | TRUE | FALSE |
| | NM_024420 | FALSE | TRUE | FALSE |
| | NM_001497 | FALSE | TRUE | FALSE |
| | NM_003574 | TRUE | TRUE | FALSE |
| | NM_006216 | TRUE | TRUE | TRUE |
| | NM_013233 | FALSE | TRUE | FALSE |
| | AK000313 | TRUE | FALSE | FALSE |
| | NM_022725 | FALSE | TRUE | FALSE |
| | NM_022780 | FALSE | TRUE | FALSE |
| | NM_032012 | FALSE | TRUE | FALSE |
| | NM_152780 | TRUE | TRUE | FALSE |
| | NM_153812 | TRUE | FALSE | FALSE |
| | NM_002078 | FALSE | TRUE | FALSE |
| | NM_003089 | TRUE | FALSE | FALSE |
| | NM_004396 | TRUE | TRUE | FALSE |
| | NM_001698 | TRUE | TRUE | TRUE |
| | NM_004568 | FALSE | TRUE | FALSE |
| C14 | NM_003677 | TRUE | TRUE | FALSE |
| | NM_018371 | TRUE | FALSE | FALSE |
| | NM_006587 | TRUE | FALSE | FALSE |
| | NM_016097 | TRUE | TRUE | FALSE |
| | NM_015224 | TRUE | FALSE | FALSE |
| | NM_020726 | TRUE | FALSE | FALSE |
| | NM_004436 | TRUE | FALSE | FALSE |
| | NM_021158 | TRUE | TRUE | FALSE |
| | AK056178 | TRUE | FALSE | FALSE |
| | NM_015134 | TRUE | FALSE | FALSE |
| | NM_016059 | TRUE | FALSE | FALSE |
| | NM_006600 | TRUE | TRUE | FALSE |
| | ENST00000307767 | TRUE | TRUE | FALSE |
| | NM_004550 | TRUE | TRUE | FALSE |
| | NM_024329 | TRUE | FALSE | FALSE |
| | NM_017845 | TRUE | TRUE | FALSE |
| | BC039726 | TRUE | FALSE | FALSE |
| | NM_001554 | TRUE | TRUE | FALSE |
| | AK057783 | TRUE | FALSE | FALSE |
| | NM_007222 | TRUE | FALSE | FALSE |
| | NM_199133 | TRUE | FALSE | FALSE |
| | Z24727 | TRUE | FALSE | FALSE |
| | NM_001765 | TRUE | FALSE | FALSE |
| | NM_005012 | TRUE | TRUE | FALSE |
| | NM_000092 | TRUE | FALSE | FALSE |
| | NM_000356 | TRUE | FALSE | FALSE |
| | NM_001516 | TRUE | FALSE | FALSE |
| | NM_002816 | TRUE | FALSE | FALSE |
| | NM_002826 | TRUE | TRUE | FALSE |
| | NM_002840 | TRUE | TRUE | FALSE |
| | NM_004287 | TRUE | FALSE | FALSE |
| | NM_005414 | TRUE | FALSE | FALSE |
| | NM_015532 | TRUE | FALSE | FALSE |
| | NM_015650 | TRUE | FALSE | FALSE |
| | NM_016341 | TRUE | FALSE | FALSE |
| | NM_181354 | TRUE | FALSE | FALSE |
| | NM_018979 | TRUE | FALSE | FALSE |
| | NM_022121 | TRUE | TRUE | FALSE |
| | NM_024699 | TRUE | FALSE | FALSE |
| | NM_032690 | TRUE | FALSE | FALSE |
| | NM_134428 | TRUE | FALSE | FALSE |
| | NM_152437 | TRUE | TRUE | FALSE |
| | NM_001168 | TRUE | FALSE | FALSE |
| | ENST00000269463 | TRUE | FALSE | FALSE |
| | NM_005647 | TRUE | FALSE | FALSE |
| | NM_016441 | TRUE | TRUE | TRUE |
| C2 | NM_014342 | TRUE | FALSE | FALSE |
| | NM_014517 | TRUE | TRUE | FALSE |
| | BX538238 | TRUE | FALSE | FALSE |
| | NM_001755 | TRUE | TRUE | TRUE |
| | NM_004433 | TRUE | FALSE | FALSE |
| | NM_016131 | TRUE | TRUE | TRUE |
| | NM_024054 | TRUE | TRUE | TRUE |
| | NM_145808 | TRUE | FALSE | TRUE |
| | A_23_P60699 | TRUE | TRUE | FALSE |
| | AL832848 | TRUE | FALSE | FALSE |
| | NM_032783 | TRUE | TRUE | TRUE |
| | NM_000117 | TRUE | FALSE | FALSE |
| | NM_001412 | TRUE | TRUE | TRUE |
| | NM_001933 | TRUE | FALSE | FALSE |
| | NM_012106 | TRUE | TRUE | TRUE |
| | NM_014316 | TRUE | FALSE | FALSE |
| | NM_001710 | TRUE | FALSE | FALSE |
| | NM_006457 | TRUE | FALSE | FALSE |
| | NM_006016 | TRUE | TRUE | TRUE |
| | NM_145058 | TRUE | TRUE | FALSE |
| | NM_018471 | TRUE | FALSE | FALSE |
| | NM_003211 | TRUE | FALSE | FALSE |
| | NM_002901 | TRUE | TRUE | TRUE |
| | NM_014888 | TRUE | FALSE | FALSE |
| | NM_005629 | TRUE | FALSE | FALSE |
| | NM_001549 | TRUE | FALSE | FALSE |
| | NM_013354 | TRUE | TRUE | TRUE |
| | NM_013994 | TRUE | FALSE | FALSE |
| | AB020721 | TRUE | FALSE | FALSE |
| | NM_014891 | TRUE | TRUE | FALSE |
| | NM_016090 | TRUE | FALSE | FALSE |
| | AK098212 | TRUE | TRUE | TRUE |
| | NM_022469 | TRUE | FALSE | FALSE |
| | NM_002136 | TRUE | TRUE | TRUE |
| | NM_080655 | TRUE | FALSE | FALSE |
| | NM_138358 | TRUE | TRUE | TRUE |
| | BC021238 | TRUE | FALSE | FALSE |
| | NM_173705 | TRUE | FALSE | FALSE |
| | NM_173714 | TRUE | FALSE | FALSE |
| | NM_004318 | TRUE | FALSE | FALSE |
| | NM_005079 | TRUE | TRUE | TRUE |
| | NM_021990 | TRUE | FALSE | FALSE |
| | NM_002245 | TRUE | TRUE | FALSE |
| | U79751 | TRUE | TRUE | FALSE |
| | NM_002273 | TRUE | TRUE | TRUE |
| C3 | NM_005467 | TRUE | FALSE | FALSE |
| | NM_007219 | TRUE | FALSE | FALSE |
| | NM_005359 | TRUE | FALSE | FALSE |
| | NM_018464 | TRUE | TRUE | FALSE |
| | THC1978535 | TRUE | TRUE | FALSE |
| | BC035054 | TRUE | FALSE | FALSE |
| | NM_014300 | TRUE | TRUE | TRUE |
| | AB014585 | TRUE | FALSE | FALSE |
| | NM_017798 | TRUE | TRUE | FALSE |
| | BC007917 | TRUE | FALSE | FALSE |
| | NM_033503 | TRUE | FALSE | FALSE |
| | NM_152898 | TRUE | TRUE | TRUE |
| C4 | NM_015927 | TRUE | FALSE | TRUE |
| | NM_018492 | TRUE | TRUE | TRUE |
| | NM_016639 | TRUE | FALSE | FALSE |
| | NM_002815 | TRUE | FALSE | FALSE |
| | NM_004386 | TRUE | TRUE | TRUE |
| | NM_006464 | TRUE | TRUE | FALSE |
| | NM_001047 | TRUE | FALSE | FALSE |
| | NM_012428 | TRUE | TRUE | TRUE |
| | BC033809 | TRUE | TRUE | FALSE |
| | NM_032026 | TRUE | FALSE | FALSE |
| | NM_016436 | TRUE | FALSE | TRUE |
| | NM_022083 | TRUE | FALSE | FALSE |
| | NM_018018 | TRUE | TRUE | FALSE |
| | A_23_P67028 | TRUE | TRUE | FALSE |
| | BC013629 | TRUE | FALSE | FALSE |
| | NM_013397 | TRUE | FALSE | TRUE |
| | NM_012091 | TRUE | TRUE | FALSE |
| | NM_030980 | TRUE | FALSE | FALSE |
| | NM_020898 | TRUE | FALSE | FALSE |
| | THC1990950 | TRUE | FALSE | FALSE |
| | NM_006818 | TRUE | FALSE | TRUE |
| | NM_012388 | TRUE | FALSE | FALSE |
| | NM_001753 | TRUE | FALSE | TRUE |
| | NM_178129 | TRUE | FALSE | FALSE |
| | NM_020374 | TRUE | TRUE | TRUE |
| | NM_003739 | TRUE | TRUE | TRUE |
| | NM_000691 | TRUE | TRUE | FALSE |
| | NM_006835 | TRUE | FALSE | FALSE |
| | NM_206858 | TRUE | TRUE | TRUE |
| | NM_022145 | TRUE | FALSE | FALSE |
| | NM_000104 | TRUE | TRUE | TRUE |
| | NM_005168 | TRUE | TRUE | FALSE |
| | A_23_P84016 | TRUE | TRUE | FALSE |
| | NM_002444 | TRUE | TRUE | FALSE |
| | NM_016302 | TRUE | TRUE | TRUE |
| | BC025376 | TRUE | FALSE | FALSE |
| | NM_021258 | TRUE | FALSE | FALSE |
| | NM_003472 | TRUE | TRUE | FALSE |
| | NM_000088 | TRUE | TRUE | FALSE |
| | NM_174887 | TRUE | TRUE | FALSE |
| | NM_031954 | TRUE | TRUE | TRUE |
| | NM_002061 | TRUE | TRUE | TRUE |
| | NM_004788 | TRUE | FALSE | FALSE |
| | NM_001387 | TRUE | TRUE | TRUE |
| | NM_001086 | TRUE | TRUE | FALSE |
| | NM_004470 | TRUE | TRUE | TRUE |
| | NM_005231 | TRUE | FALSE | FALSE |
| | NM_000189 | TRUE | TRUE | FALSE |
| | NM_001535 | TRUE | TRUE | FALSE |
| | NM_001660 | TRUE | TRUE | TRUE |
| | NM_001754 | TRUE | FALSE | FALSE |
| | NM_002094 | TRUE | FALSE | FALSE |
| | NM_003286 | TRUE | FALSE | FALSE |
| | NM_016823 | TRUE | TRUE | FALSE |
| | NM_006764 | TRUE | TRUE | TRUE |
| | NM_012383 | TRUE | FALSE | FALSE |
| | AK000796 | TRUE | FALSE | FALSE |
| | NM_018132 | TRUE | TRUE | TRUE |
| | NM_018390 | TRUE | TRUE | TRUE |
| | NM_020314 | TRUE | FALSE | FALSE |
| | NM_021156 | TRUE | TRUE | TRUE |
| | NM_022074 | TRUE | FALSE | FALSE |
| | NM_032132 | TRUE | FALSE | FALSE |
| | NM_080546 | TRUE | TRUE | TRUE |
| | NM_080725 | TRUE | TRUE | TRUE |
| | NM_080927 | TRUE | TRUE | FALSE |
| | NM_152344 | TRUE | TRUE | FALSE |
| | NM_152523 | TRUE | TRUE | FALSE |
| | NM_000408 | TRUE | TRUE | TRUE |
| | NM_003675 | TRUE | TRUE | TRUE |
| | NM_001425 | TRUE | TRUE | FALSE |
| | NM_006825 | TRUE | TRUE | TRUE |
| | NM_022360 | TRUE | FALSE | FALSE |
| C52 | AB011134 | TRUE | FALSE | FALSE |
| | NM_002705 | TRUE | FALSE | FALSE |
| | NM_002317 | TRUE | FALSE | FALSE |
| | NM_006594 | TRUE | FALSE | FALSE |
| | NM_018004 | TRUE | FALSE | FALSE |
| | AL137442 | TRUE | FALSE | FALSE |
| | NM_024071 | TRUE | FALSE | FALSE |
| | NM_002925 | TRUE | FALSE | FALSE |
| | NM_006773 | TRUE | FALSE | FALSE |
| | NM_003370 | TRUE | FALSE | FALSE |
| | NM_052859 | TRUE | FALSE | FALSE |
| | NM_014344 | TRUE | FALSE | FALSE |
| | NM_006285 | TRUE | FALSE | FALSE |
| | NM_000303 | TRUE | FALSE | FALSE |
| | NM_000723 | TRUE | FALSE | FALSE |
| | NM_003731 | TRUE | FALSE | FALSE |
| | NM_004042 | TRUE | FALSE | FALSE |
| | NM_004354 | TRUE | FALSE | FALSE |
| | NM_005417 | TRUE | FALSE | FALSE |
| | NM_012207 | TRUE | FALSE | FALSE |
| | NM_014298 | TRUE | FALSE | FALSE |
| | NM_015947 | TRUE | FALSE | FALSE |
| | NM_016479 | TRUE | FALSE | FALSE |
| | NM_017590 | TRUE | FALSE | FALSE |
| | NM_018685 | TRUE | FALSE | FALSE |
| | NM_020188 | TRUE | FALSE | FALSE |
| | NM_025147 | TRUE | FALSE | FALSE |
| | NM_025198 | TRUE | FALSE | FALSE |
| | NM_032620 | TRUE | FALSE | FALSE |
| | NM_033502 | TRUE | FALSE | FALSE |
| | NM_145110 | TRUE | FALSE | FALSE |
| | THC1943229 | TRUE | FALSE | FALSE |
| | NM_173607 | TRUE | FALSE | FALSE |
| | NM_000389 | TRUE | FALSE | FALSE |
| | THC1961572 | TRUE | FALSE | FALSE |
| | NM_004380 | TRUE | FALSE | FALSE |
| | NM_002857 | TRUE | FALSE | FALSE |
| G4 | NM_198278 | TRUE | FALSE | FALSE |
| | NM_015584 | TRUE | TRUE | FALSE |
| | NM_002720 | TRUE | TRUE | FALSE |
| | AY359048 | FALSE | TRUE | FALSE |
| | NM_005349 | TRUE | FALSE | FALSE |
| | D14041 | TRUE | TRUE | FALSE |
| G41 | NM_033520 | TRUE | FALSE | FALSE |
| | NM_006554 | TRUE | TRUE | FALSE |
| | NM_016441 | TRUE | TRUE | FALSE |
| | NM_022163 | TRUE | FALSE | FALSE |
| | NM_020381 | TRUE | TRUE | FALSE |
| | NM_002109 | TRUE | FALSE | FALSE |
| | NM_013402 | TRUE | FALSE | FALSE |
| | NM_033515 | TRUE | FALSE | FALSE |
| | NM_004060 | TRUE | FALSE | FALSE |
| | NM_004096 | TRUE | TRUE | FALSE |
| | NM_017946 | TRUE | FALSE | FALSE |
| | NM_002524 | TRUE | TRUE | FALSE |
| | NM_002834 | TRUE | FALSE | FALSE |
| | A_23_P165819 | TRUE | TRUE | FALSE |
| | BC029424 | TRUE | TRUE | FALSE |
| | D31887 | TRUE | FALSE | FALSE |
| | NM_001387 | TRUE | TRUE | FALSE |
| | NM_001921 | TRUE | TRUE | FALSE |
| | NM_007096 | TRUE | TRUE | FALSE |
| | NM_001349 | TRUE | TRUE | FALSE |
| | NM_001743 | TRUE | TRUE | FALSE |
| | NM_001943 | TRUE | TRUE | FALSE |
| | NM_002721 | TRUE | TRUE | FALSE |
| | NM_003501 | TRUE | TRUE | FALSE |
| | NM_004261 | TRUE | FALSE | FALSE |
| | NM_006759 | TRUE | TRUE | FALSE |
| | NM_018046 | TRUE | TRUE | FALSE |
| | NM_018192 | TRUE | TRUE | FALSE |
| | NM_032132 | TRUE | FALSE | FALSE |
| | NM_052839 | TRUE | FALSE | FALSE |
| | NM_002190 | TRUE | FALSE | FALSE |
| | ENST00000328742 | TRUE | TRUE | FALSE |
| | NM_002346 | TRUE | TRUE | FALSE |
| | NM_002133 | TRUE | TRUE | FALSE |
| | NM_001628 | TRUE | TRUE | FALSE |
| | NM_000138 | TRUE | FALSE | FALSE |
| M1 | NM_015055 | TRUE | FALSE | FALSE |
| | NM_016047 | TRUE | TRUE | TRUE |
| | NM_018250 | TRUE | TRUE | TRUE |
| | NM_138467 | TRUE | TRUE | TRUE |
| | NM_017845 | TRUE | TRUE | FALSE |
| | NM_005567 | TRUE | TRUE | TRUE |
| | NM_006345 | TRUE | TRUE | FALSE |
| | NM_001724 | TRUE | FALSE | FALSE |
| | NM_021913 | TRUE | TRUE | TRUE |
| | NM_005895 | TRUE | TRUE | FALSE |
| | NM_005349 | TRUE | FALSE | FALSE |
| | NM_006711 | TRUE | FALSE | TRUE |
| | NM_001087 | TRUE | TRUE | TRUE |
| | NM_002185 | TRUE | TRUE | TRUE |
| | NM_012347 | TRUE | FALSE | FALSE |
| | NM_014033 | TRUE | FALSE | FALSE |
| | NM_014889 | TRUE | TRUE | TRUE |
| | NM_001981 | TRUE | TRUE | FALSE |
| | NM_032122 | TRUE | TRUE | TRUE |
| | NM_005877 | TRUE | TRUE | TRUE |
| | NM_153812 | TRUE | FALSE | FALSE |
| | NM_004311 | TRUE | TRUE | TRUE |
| | NM_001379 | TRUE | TRUE | TRUE |
| | NM_001494 | TRUE | TRUE | FALSE |
| M2 | NM_014908 | TRUE | FALSE | TRUE |
| | NM_020062 | TRUE | TRUE | TRUE |
| | NM_018686 | TRUE | TRUE | FALSE |
| | NM_021238 | TRUE | FALSE | FALSE |
| | NM_004965 | TRUE | TRUE | TRUE |
| | NM_014374 | TRUE | TRUE | TRUE |
| | NM_014670 | TRUE | FALSE | FALSE |
| | NM_018429 | TRUE | TRUE | FALSE |
| | NM_020470 | TRUE | TRUE | FALSE |
| | NM_020820 | TRUE | FALSE | FALSE |
| | NM_004731 | TRUE | TRUE | FALSE |
| M3 | NM_078470 | TRUE | TRUE | TRUE |
| | NM_032574 | TRUE | TRUE | TRUE |
| | NM_001948 | TRUE | FALSE | FALSE |
| | NM_002657 | TRUE | FALSE | FALSE |
| | NM_012249 | TRUE | TRUE | FALSE |
| | NM_152344 | TRUE | TRUE | FALSE |
| M4 | AB002370 | TRUE | FALSE | FALSE |
| | NM_004844 | TRUE | TRUE | FALSE |
| | NM_015455 | TRUE | FALSE | TRUE |
| | NM_016542 | TRUE | TRUE | FALSE |
| | NM_001262 | TRUE | TRUE | FALSE |
| | NM_198969 | TRUE | FALSE | FALSE |
| | NM_012428 | TRUE | TRUE | TRUE |
| | NM_013372 | TRUE | TRUE | TRUE |
| | NM_013237 | TRUE | TRUE | TRUE |
| | NM_014071 | TRUE | TRUE | FALSE |
| | NM_014112 | TRUE | FALSE | FALSE |
| | NM_022740 | TRUE | TRUE | TRUE |
| | NM_138444 | TRUE | FALSE | TRUE |
| | BC032468 | TRUE | FALSE | FALSE |
| | NM_015134 | TRUE | FALSE | TRUE |
| | NM_000691 | TRUE | FALSE | FALSE |
| | NM_002902 | TRUE | TRUE | TRUE |
| | NM_022149 | TRUE | TRUE | FALSE |
| | NM_016619 | TRUE | FALSE | FALSE |
| | NM_002960 | TRUE | TRUE | TRUE |
| | NM_031286 | TRUE | TRUE | TRUE |
| | NM_003472 | TRUE | TRUE | TRUE |
| | NM_032124 | TRUE | TRUE | TRUE |
| | NM_014615 | TRUE | FALSE | FALSE |
| | NM_003200 | TRUE | TRUE | TRUE |
| | NM_004120 | TRUE | TRUE | FALSE |
| | NM_021137 | TRUE | FALSE | FALSE |
| | NM_006756 | TRUE | TRUE | TRUE |
| | NM_002224 | TRUE | FALSE | FALSE |
| | NM_005120 | TRUE | TRUE | FALSE |
| | NM_006628 | TRUE | TRUE | TRUE |
| | NM_012207 | TRUE | TRUE | TRUE |
| | NM_016516 | TRUE | TRUE | FALSE |
| | NM_025075 | TRUE | TRUE | FALSE |
| | NM_031427 | TRUE | FALSE | FALSE |
| | NM_004176 | TRUE | TRUE | FALSE |
| | THC1811009 | TRUE | FALSE | FALSE |
| | NM_002522 | TRUE | FALSE | FALSE |
| | NM_139045 | TRUE | TRUE | TRUE |

TABLE II

| Validated | | Predicted* | | | |
|---|---|---|---|---|---|
| siRNA | Off-Targets | ≧79% | ≧84% | ≧89% | ≧95% but <100% |
| c1 | 13 | 917 | 66 | 2 | 0 |
| c2 | 46 | 831 | 105 | 3 | 0 |
| c3 | 12 | 890 | 64 | 1 | 0 |
| c4 | 73 | 806 | 147 | 8 | 0 |
| c14 | 45 | 920 | 84 | 2 | 0 |
| c52 | 37 | 913 | 102 | 9 | 0 |
| g4 | 5 | 896 | 74 | 2 | 0 |
| g41 | 36 | 899 | 88 | 5 | 1 |
| m1 | 24 | 933 | 123 | 9 | 1 |
| m2 | 10 | 935 | 180 | 8 | 0 |
| m3 | 7 | 920 | 112 | 3 | 0 |
| m4 | 39 | 892 | 133 | 2 | 0 |

*Predicted target number based on overall percentage identity

TABLE III

| | | | Gap | |
|---|---|---|---|---|
| Id | Matches | Mismatches | Open | Gap Extend |
| 1 | Watson-Crick = 1 | All = −1 | 0 | −1 |
| 2 | Watson-Crick = 1 | All = −1 | 9 | −10 |
| 3 | Watson-Crick = 1 | All = −1 | 0 | −3 |
| 4 | Watson-Crick = 1 | All = −1 | 9 | −12 |
| 5 | Watson-Crick = 1 | All = −1 | 0 | −1 |
| | GU/UG = 1 | | | |
| 6 | Watson-Crick = 1 | All = −1 | 9 | −10 |
| | GU/UG = 1 | | | |
| 7 | Watson-Crick = 1 | All = −1 | 0 | −3 |
| | GU/UG = 1 | | | |
| 8 | Watson-Crick = 1 | All = −1 | 9 | −12 |
| | GU/UG = 1 | | | |
| 9 | Watson-Crick = 2 | All = −1 | 0 | −1 |
| | GU/UG = 1 | | | |
| 10 | Watson-Crick = 2 | All = −1 | 9 | −10 |
| | GU/UG = 1 | | | |
| 11 | Watson-Crick = 2 | All but GA = −1 | 0 | −2 |
| | GU/UG = 1 | GA = −2 | | |
| 12 | Watson-Crick = 2 | All but GA = −1 | 9 | −11 |
| | GU/UG = 1 | GA = −2 | | |
| 13 | Watson-Crick = 1 | All = −1 | 0 | −1 |
| | AC = 1 | | | |
| 14 | Watson-Crick = 1 | All = −1 | 9 | −10 |
| | AC = 1 | | | |
| 15 | Watson-Crick = 2 | All = −1 | 0 | −1 |
| | AC = 1 | | | |
| 16 | Watson-Crick = 2 | All = −1 | 9 | −10 |
| | AC = 1 | | | |
| 17 | Watson-Crick = 1 | All = −1 | 0 | −1 |
| | GU/UG/AC = 1 | | | |
| 18 | Watson-Crick = 1 | All = −1 | 9 | −10 |
| | GU/UG/AC = 1 | | | |
| 19 | Watson-Crick = 2 | All = −1 | 0 | −1 |
| | GU/UG/AC = 1 | | | |
| 20 | Watson-Crick = 2 | All = −1 | 9 | −10 |
| | GU/UG/AC = 1 | | | |
| 21 | Watson-Crick = 1 | All = −1 | 0 | −1 |
| | GU/UG/AC/CA = 1 | | | |
| 22 | Watson-Crick = 1 | All = −1 | 9 | −10 |
| | GU/UG/AC/CA = 1 | | | |
| 23 | Watson-Crick = 4 | All = −1 | 0 | −1 |
| | GU/UG = 2 | | | |
| | AC/CA = 1 | | | |
| 24 | Watson-Crick = 4 | All = −1 | 9 | −10 |
| | GU/UG = 2 | | | |
| | AC/CA = 1 | | | |
| 25 | Watson-Crick = 4 | GA = −4 | 0 | −4 |
| | GU/UG = 2 | AA/AG/CC/GG = −2 | | |
| | AC/CA = 1 | CU/UC/UU = −1 | | |
| 26 | Watson-Crick = 4 | GA = −4 | 9 | −13 |
| | GU/UG = 2 | AA/AG/CC/GG = −2 | | |
| | AC/CA = 1 | CU/UC/UU = −1 | | |
| 27 | Watson-Crick = 4 | GA = −4 | 0 | −6 |
| | GU/UG = 2 | AA/AG/CC/GG = −2 | | |
| | AC/CA = 1 | CU/UC/UU = −1 | | |
| 28 | Watson-Crick = 4 | GA = −4 | 9 | −15 |
| | GU/UG = 2 | AA/AG/CC/GG = −2 | | |
| | AC/CA = 1 | CU/UC/UU = −1 | | |
| 29 | Watson-Crick = 4 | GA = −4 | 0 | −4 |
| | GU/UG = 2 | AA/AG/CC/GG = −2 | | |
| | AC/CA/CU/UC = 1 | UU = −1 | | |
| 30 | Watson-Crick = 4 | GA = −4 | 9 | −13 |
| | GU/UG = 2 | AA/AG/CC/GG = −2 | | |
| | AC/CA/CU/UC = 1 | UU = −1 | | |

TABLE IV

| | | | | | | | Positive |
|---|---|---|---|---|---|---|---|
| | | | | False | | | Predictive Power |
| Criteria | True Positives | False Positives | True Negatives | Negatives | Specificity (%) | Specificity (%) | (%) |
| At least 1 | 71 | 15 | 69 | 13 | 85 | 82 | 83 |
| hexamer in | | | | | | | |
| 3′ UTR | | | | | | | |
| At least 2 | 25 | 3 | 81 | 59 | 30 | 96 | 89 |
| hexamer in | | | | | | | |
| 3′ UTR | | | | | | | |
| At least 3 | 6 | 1 | 83 | 78 | 7 | 99 | 86 |
| hexamer in | | | | | | | |
| 3′ UTR | | | | | | | |
| At least 4 | 4 | 0 | 84 | 80 | 5 | 100 | 100 |
| hexamer in | | | | | | | |
| 3′ UTR | | | | | | | |
| At least 1 | 58 | 7 | 77 | 26 | 69 | 92 | 89 |
| heptamer in | | | | | | | |
| 3′ UTR | | | | | | | |
| At least 2 | 8 | 0 | 84 | 76 | 10 | 100 | 100 |
| heptamer in | | | | | | | |
| 3′ UTR | | | | | | | |
| At least 3 | 1 | 0 | 84 | 83 | 1 | 100 | 100 |
| heptamer in | | | | | | | |
| 3′ UTR | | | | | | | |
| At least 4 | 0 | 0 | 84 | 84 | 0 | 0 | NA |
| heptamer in | | | | | | | |
| 3′ UTR | | | | | | | |

TABLE V

1081 low frequency hexamer sequences
distinctnmutr3s: number of 3′ UTRs
in which the sequence appears at least
once

motif

| | |
|---|---|
| GCAGCG | 1966 |
| ATATCG | 621 |
| CAATCG | 562 |
| TCGGAT | 678 |
| GTGACG | 1241 |
| CCGCAT | 1058 |
| CACGAT | 1036 |
| GACGCT | 1069 |
| CGTCCG | 465 |
| CGAAGG | 1136 |
| GTTGCG | 720 |
| GCCGTT | 1097 |
| ACGCGC | 456 |
| ACCGAC | 743 |
| TGTGCG | 1673 |
| TCGTTA | 761 |
| TTTCGA | 1013 |
| TAATCG | 652 |
| GCGCCT | 1875 |
| GCCGAT | 662 |
| TCGGTT | 1046 |
| TACGAT | 665 |
| GTCCGC | 756 |
| AGCTCG | 1102 |
| TCGATG | 908 |
| TCACCG | 1516 |
| TTCGGA | 995 |
| CAAGCG | 1239 |
| CACGTT | 1798 |
| AACGGC | 736 |
| ATAGCG | 615 |
| GGTCGC | 662 |
| TCTCGC | 1306 |
| AGTTCG | 1047 |
| CGACCT | 1063 |
| TGCCGG | 1636 |
| TTGGCG | 1029 |
| GAGTCG | 908 |
| AGCCCG | 1833 |
| CCGCTT | 1366 |
| AACACG | 1404 |
| AGGAGA | 1050 |
| CCACGA | 1396 |
| AGCGGA | 1135 |
| CGCTCC | 1682 |
| CTTCGA | 986 |
| AGGGCG | 1598 |
| ATCCGT | 903 |
| TGCGCC | 1556 |
| TCGCAA | 547 |
| TTCTCG | 1385 |
| AGACGC | 1165 |
| GCGATT | 989 |
| AGGCGA | 1105 |
| AGCGAA | 957 |
| CATCGT | 1250 |
| GACCGA | 917 |
| CGTTCC | 1364 |
| TTCCCG | 1846 |
| CGGGCC | 1926 |
| GCGGAA | 1004 |
| CTCTCG | 1542 |
| CGATTA | 555 |
| CGTCAC | 1073 |
| CGCAGT | 1229 |
| CATTCG | 884 |
| TACGTT | 1265 |
| CGAGAA | 1248 |
| CGTACA | 704 |
| CCATCG | 1240 |
| ACCGCG | 599 |
| GCCGCT | 1582 |
| GATCGG | 582 |
| GAAACG | 1523 |
| ACGTGC | 1765 |
| CTCGGA | 1329 |
| TAAGCG | 606 |
| TCGACC | 611 |
| TATCGT | 774 |
| CGCGGG | 896 |
| AGTCGT | 937 |
| GGACCG | 1148 |
| CGCACA | 1444 |
| CTGGCG | 1788 |
| CGGATA | 462 |
| CGTAGC | 756 |
| TCGGCC | 1828 |
| GCGTCG | 350 |
| ACCGGC | 1040 |
| CGGCAG | 1914 |
| TACGCC | 556 |
| ACCACG | 1808 |
| ACGCTA | 572 |
| TCGCTG | 1754 |
| CGCGCA | 513 |
| GTATCG | 549 |
| CGTGAA | 1584 |
| GACGCG | 398 |
| GCCCGA | 1271 |
| AACGTA | 1029 |
| AGTCGG | 1003 |
| GCGGGA | 1648 |
| AAGCGT | 1105 |
| CCGAGT | 1553 |
| CGAAAG | 1005 |
| CGAGTG | 1262 |
| ACTACG | 580 |
| GCGCCG | 670 |
| AATCGA | 838 |
| TTCGAA | 962 |
| TTGCGA | 679 |
| CCGACA | 1049 |
| GCGCAC | 914 |
| TCGTTC | 1045 |
| TAACGA | 675 |
| CGACTT | 953 |
| ACGCTC | 987 |
| CGCGGT | 584 |
| ACGTAT | 1155 |
| GCAACG | 792 |
| ATAACG | 722 |
| TTACGG | 757 |
| AACGTC | 1000 |
| TCCGTG | 1911 |
| CAACGA | 742 |
| CGACAT | 796 |
| CTGCGA | 1188 |
| TGTCGA | 736 |
| TCCGGG | 1531 |
| ATCCGG | 737 |
| CGCGAG | 366 |
| CGGCGG | 855 |
| CGATTC | 1067 |
| GCGAAA | 843 |
| CTCGAA | 1276 |
| GTACGA | 502 |
| GAGCGC | 1098 |
| CGGTAC | 501 |
| CCGAAG | 1359 |
| CTACGG | 651 |
| GACGAC | 654 |
| CCGGTG | 1457 |
| AGTCGC | 688 |
| CGTCTT | 1642 |
| TCGTGG | 1525 |
| CGTAAC | 588 |
| ACGGAA | 1292 |
| AACCGA | 908 |
| CGCGTC | 457 |
| CCGGGT | 1721 |
| TCGTAC | 519 |
| AAGCCG | 1388 |
| GGCGAA | 841 |
| GGGCGA | 1269 |
| ACGATT | 981 |
| GGACGC | 1179 |
| CGCAAC | 557 |
| TCCGCA | 1122 |
| TGACGG | 1176 |
| CGGTGT | 1248 |
| AGACCG | 1089 |
| GCGTGC | 1477 |
| CCGGAG | 1806 |
| GGTCGT | 762 |
| TCCGGT | 795 |
| CGGTCA | 913 |
| AATCGG | 756 |
| GCCGCG | 862 |
| ACCGCT | 1043 |
| CGCGTA | 140 |
| TATCGC | 463 |
| ACATCG | 925 |
| TACCGG | 585 |
| CGGCGT | 465 |
| TGCCGT | 1728 |
| GTAGCG | 562 |
| GACGGC | 1086 |
| ATCCGC | 913 |
| TCTCCG | 1638 |
| CGTTAA | 928 |
| GGCTCG | 1174 |
| ACCGAT | 701 |
| ACGCCT | 1991 |
| CGATGG | 1102 |
| CACCGG | 1413 |
| CGACCC | 1065 |
| CGGATC | 986 |
| GCGCGC | 578 |
| GCCGAC | 906 |
| CGGCCA | 1790 |
| ATTGCG | 716 |
| ACCGTT | 1050 |
| CGATAC | 384 |
| CATCGC | 1042 |
| AACGCT | 1122 |
| CGCTAA | 621 |
| ATGACG | 980 |
| CGTCCT | 1817 |
| ACAGCG | 1437 |
| CGAAGT | 922 |
| GTCCGT | 1065 |
| AGCGTG | 1691 |
| TCGCGG | 357 |
| CGCAGC | 1815 |
| TCCGAG | 1362 |
| GGCGGA | 1751 |
| GCGAGA | 1258 |
| GACACG | 1284 |
| CCTCGA | 1298 |
| CGAACA | 737 |
| AAGTCG | 876 |
| CCGTCC | 1812 |
| TTACGT | 1285 |
| CGAGGG | 1739 |
| GGTTCG | 652 |
| AACGCG | 231 |
| TCCGTA | 896 |
| CTTCGG | 1427 |
| CCGGTA | 504 |
| TCGCGT | 293 |
| CTCGTG | 1777 |
| CGGCTC | 1992 |
| CGATGT | 943 |
| CACCGT | 1859 |
| GACGTC | 952 |
| CGGTAT | 567 |
| TTCGTG | 1455 |
| TACCGT | 851 |
| ACAACG | 820 |
| GTAACG | 602 |
| CGTTTG | 1684 |
| GCGTAT | 646 |
| CGATCA | 652 |
| GCGCTC | 1206 |
| TTTCGG | 1141 |
| CCGTAA | 814 |
| CTACGT | 903 |
| TCGTGT | 1588 |
| ACGCAC | 1132 |
| TGGACG | 1420 |
| CGAGGT | 1398 |
| CCGAGC | 1583 |
| AACGAC | 665 |
| AAGCGC | 877 |
| TCGATC | 627 |
| TCGCCA | 1217 |
| ATACGA | 754 |
| CGAGCA | 1170 |
| GTCCGG | 932 |
| CGGTTT | 1344 |
| ACGAAA | 1226 |
| GCGTTT | 1494 |
| CATCCG | 1073 |
| TCGATA | 518 |
| CGCACG | 482 |
| GCGCTA | 542 |
| TTCGGG | 1177 |
| GCCGGC | 1823 |
| CGCGGC | 763 |
| ACGTCG | 306 |
| GCCGTC | 1233 |
| CGAGAG | 1404 |
| TATCCG | 510 |
| CCGGCA | 1596 |
| CGTACG | 163 |
| CGTCAT | 1127 |
| GATCGA | 675 |
| ACGCCG | 466 |
| TCGCAG | 1067 |
| GCTACG | 632 |
| CGGCTA | 753 |
| GAGCGT | 1090 |
| ACGGGA | 1284 |
| GGTCGG | 1021 |
| GACGTA | 607 |
| ACCCGA | 846 |
| GCGTCA | 888 |
| CGATTT | 1344 |
| TTAACG | 942 |
| TCGAAC | 794 |
| AACGTG | 1881 |
| CTTTCG | 1237 |
| CCGACG | 415 |
| TGCGAC | 620 |
| ACGGCC | 1304 |
| TACGTC | 608 |
| CGATAT | 565 |
| CGAAAC | 914 |
| TGGCGC | 1562 |
| GGCCGC | 1947 |
| GGACGT | 1284 |
| GCGATC | 737 |
| TGCGCG | 512 |
| CGCACT | 978 |
| CAACGG | 780 |
| ACCGGG | 1221 |
| TACACG | 879 |
| GCGCCA | 1473 |
| CGGTGC | 1369 |
| GCGTGT | 1775 |
| AGTCGA | 619 |
| TCGGTC | 780 |
| CGCGCG | 384 |
| CGTGAG | 1935 |
| ATCGCT | 1333 |
| GGGACG | 1532 |
| CGGCGC | 683 |
| CGCGAC | 243 |
| TCGTAA | 806 |
| TCGGTA | 603 |
| AGCCGT | 1421 |
| GACGGT | 964 |
| AACGGG | 1066 |
| GCCGTA | 562 |
| CCGGTC | 886 |
| ATGTCG | 866 |
| CTACGC | 563 |
| TAGCGT | 726 |
| CGAGTA | 888 |
| ACTCCG | 1356 |
| TCACGG | 1342 |
| GACGCA | 985 |
| GCGCGT | 416 |
| CGTACT | 683 |
| CCGAAC | 633 |
| CGAAGC | 1085 |
| CGGAGA | 1403 |
| GTCGCC | 1119 |
| GCGCAG | 1548 |
| CTTCGT | 1442 |
| CGTCCC | 1679 |
| ATGCCG | 1113 |
| ATCCGA | 684 |
| ACGCTG | 1759 |
| CTCGAG | 1333 |
| CGCTTG | 1386 |
| GATGCG | 885 |
| CCGGAC | 1152 |
| CAACGT | 1155 |
| CGCTGA | 1289 |
| CGGTCG | 214 |
| GTCGTT | 859 |
| GCGATA | 403 |
| GACGAG | 1051 |
| CGTGTA | 1251 |
| GCTAGC | 1865 |
| TCTCGG | 1932 |
| ACGGAT | 796 |
| CGCGCT | 536 |
| TGAACG | 1157 |
| GAGCGG | 1355 |
| CGGCCG | 949 |
| CTCGGT | 1329 |
| GCCGGT | 1011 |
| TCGTTG | 956 |
| TAGCGC | 506 |
| ACGATG | 1087 |
| ACACCG | 1149 |
| ACGGTT | 1036 |
| TACGAC | 434 |
| ACGTTA | 1088 |
| AGTGCG | 1040 |
| CGTTGA | 896 |
| CGCAAT | 649 |
| CGCTAG | 531 |
| CGCCGA | 416 |
| CAGACG | 1552 |
| GGACGG | 1527 |
| CTCGCA | 1061 |
| GCCGCA | 1440 |
| TGCCGA | 1208 |
| GTTACG | 636 |
| CGATGC | 923 |
| CACCGC | 1899 |
| CCGTTG | 1090 |
| TTCCGT | 1540 |
| TCGGGC | 1186 |
| GCGTAC | 359 |
| AAACCG | 1201 |
| CGTTAG | 739 |
| CGTAAT | 795 |
| CGAACG | 204 |
| CTCGTA | 655 |
| TTAGCG | 629 |
| ACGTTC | 1152 |
| CTGCGT | 1970 |
| TCGACG | 229 |
| TACGGC | 482 |
| ACCGTG | 1872 |
| GTCGAT | 469 |
| ATCGCG | 321 |
| CGAGTC | 842 |
| CGGAAA | 1349 |
| GCGCGG | 835 |
| CGTGCA | 1762 |
| CGGCAC | 1276 |
| TCACGT | 1663 |
| ACTCGC | 907 |
| TCCCGC | 1825 |
| TTATCG | 721 |
| TCCTCG | 1720 |
| ACGATC | 649 |
| AACGCA | 1051 |
| ACGCGT | 345 |
| GCTCCG | 1638 |
| CGCTTA | 631 |
| TCTTCG | 1224 |
| GTGTCG | 970 |
| CGATCG | 164 |
| ACCGTA | 708 |
| CACCCG | 1980 |
| AACGGT | 826 |
| GACGGG | 1731 |
| CGCGAT | 284 |
| CACGGA | 1497 |
| GGCCGT | 1442 |
| TAAACG | 1326 |
| GACGTG | 1622 |
| TTACGA | 797 |
| CGTATG | 875 |
| CGTGTC | 1654 |
| CCTCGT | 1771 |
| CGCACC | 1403 |
| TATCGG | 476 |
| AATGCG | 860 |
| TCTCGT | 1291 |
| GCGCTG | 1751 |
| GTCCGA | 642 |
| CGAGCG | 402 |
| GTGCCG | 1439 |
| CGCGTT | 328 |
| CGCATG | 1177 |
| CTACCG | 702 |
| CGTTTA | 1257 |
| CGAACT | 1022 |
| ATCGCC | 836 |
| ACCGTC | 1031 |
| TCGGAC | 691 |
| CCTTCG | 1473 |
| AGACGT | 1394 |
| AGCCGC | 1705 |
| CGCCAA | 973 |
| TGGTCG | 803 |
| CGAGAC | 1671 |
| CGTACC | 534 |
| CGGGAA | 1563 |
| GCGGCC | 1808 |
| CTCGTC | 1141 |
| CCGACT | 1098 |
| TCGGCG | 382 |
| GAACCG | 944 |
| ACGTCA | 1204 |
| CCCGGA | 1736 |
| AGGACG | 1562 |
| CATACG | 724 |
| TCGACT | 742 |
| CTTCGC | 1045 |
| GTCGCT | 858 |
| TCCGGA | 1147 |
| GGTCGA | 508 |
| GGGATT | 759 |
| ACGCCA | 1308 |
| TGCGCT | 1258 |
| CCGGCG | 825 |
| TACGCG | 170 |
| GTCGCG | 278 |
| CAGCGA | 1430 |
| CACGAA | 1129 |
| TTTGCG | 1057 |
| ACCGGT | 594 |
| TACGCT | 642 |
| CAACGC | 691 |
| CGGCAT | 968 |
| CCGCAA | 892 |
| CGCGCC | 964 |
| CGTGAC | 1195 |
| GCGTTC | 922 |
| TCGTGA | 1279 |
| TTGACG | 826 |
| CGACGA | 258 |
| ACGTAC | 700 |
| TGACGA | 902 |
| TATTCG | 682 |
| CGAAAT | 936 |
| GCTCGC | 991 |
| TTCCGC | 1080 |
| CGGCTT | 1362 |
| TCGGCT | 1630 |
| ACGCGG | 493 |
| ACCGAG | 1387 |
| ACGCAG | 1492 |
| TGCGAT | 887 |
| GGTGCG | 1249 |
| GCGTTA | 643 |
| TAGCCG | 962 |
| ATCGAT | 768 |
| GCACCG | 1349 |
| GCGATG | 913 |
| CCGTGA | 1634 |
| CGTTTC | 1813 |
| TACCGA | 684 |
| CTTCCG | 1608 |
| AAGCGG | 1178 |
| GCGGAT | 981 |
| CTGCGC | 1733 |
| CTCGAC | 826 |
| ACGATA | 571 |
| CCGGCT | 1993 |
| AACGAG | 982 |
| TGAGCG | 1293 |
| TGCGTT | 1340 |
| CGCTTC | 1377 |
| ATCGTT | 1058 |
| GCGACC | 725 |
| CGGTCT | 987 |
| CCGAAT | 869 |
| CCGTAG | 820 |
| CCGCGA | 341 |
| CCCGAA | 1180 |
| TAGTCG | 467 |
| ATTACG | 769 |
| CACTCG | 1230 |
| TCGCGA | 165 |
| TCCGAA | 971 |
| AGACGG | 1922 |
| ACCGCA | 1157 |
| GCGGTT | 811 |
| TGATCG | 814 |
| TCACGC | 1796 |
| TCGAAT | 820 |
| TCGTAG | 654 |
| GAACGC | 869 |
| CTCGCG | 414 |
| AGCCGA | 1636 |
| CGAGTT | 1010 |
| CGCTAC | 513 |
| GACGAA | 781 |
| GAGCGA | 1256 |
| CGAATG | 967 |
| ATGCGT | 1061 |
| ATCGTA | 696 |
| TTCGCG | 230 |
| CGAGAT | 1293 |
| AGAACG | 1316 |
| GCGCAA | 624 |
| CCGTTC | 1136 |
| TCGAGG | 1316 |
| GGCGCC | 1921 |
| GTCGGC | 813 |
| TCACGA | 1085 |
| CCTCGC | 1843 |
| ACTCGG | 1506 |
| CGCCGG | 734 |
| CGAACC | 610 |
| GCGGCT | 1497 |
| CGGACA | 1101 |
| GGACGA | 1000 |
| TAACCG | 614 |
| CGTTAC | 624 |
| CGTTGG | 1132 |
| AGCGCT | 1345 |
| GCGTGA | 1648 |
| AATACG | 1083 |
| GTTCCG | 902 |
| CGTGCG | 549 |
| CCGTTA | 704 |
| CGATCT | 1063 |
| TCAGCG | 1445 |
| GTCGAC | 374 |
| TCCGTT | 1250 |
| GTGCGC | 1056 |
| CGGAGT | 1216 |
| CGACAA | 707 |
| ACGGAC | 919 |
| CCGGAT | 857 |
| GCGCGA | 356 |
| GCCGAA | 946 |
| TTCCGA | 1044 |
| CGGAAG | 1522 |
| AACCGC | 751 |
| CGGGTG | 1954 |
| GCGAAT | 628 |
| AGGTCG | 930 |
| GCACGC | 1317 |
| GCGTAG | 574 |
| TCGTCT | 1251 |
| CCGACC | 1134 |
| CGAGCT | 1152 |
| TGCGGG | 1653 |
| TTGCCG | 1140 |
| ACGTTG | 1311 |
| ATCGCA | 837 |
| TCATCG | 1005 |
| CCGGTT | 895 |
| CCGATG | 985 |
| TCGCCT | 1424 |
| GACTCG | 1099 |
| TCCGAT | 628 |
| AAGACG | 1342 |
| TTGTCG | 834 |
| AAACGG | 1302 |
| GTACCG | 561 |
| ATCGGT | 624 |
| GGCGTT | 1058 |
| ATACGC | 548 |
| CGTATC | 680 |
| ACGAAC | 629 |
| TCTGCG | 1507 |
| ACGGTC | 775 |
| GGCGAT | 688 |
| GACGGA | 1255 |
| CACGGG | 1816 |
| CTGTCG | 1544 |
| CGAGCC | 1420 |
| AGCGAC | 791 |
| AGGCGC | 1532 |
| GACCCG | 1172 |
| GGATCG | 805 |
| CGGGGT | 1833 |
| CGCCGT | 577 |
| TCGACA | 709 |
| CGTGCT | 1775 |
| CTCCGA | 1249 |
| TGCGCA | 1051 |
| CGCCAG | 1817 |
| TCGGGG | 1804 |
| GCTCGT | 885 |
| ATGCGG | 903 |
| ATCGAG | 943 |
| TCGAGT | 800 |
| GGAGCG | 1536 |
| TGCGGT | 1305 |
| TTCGCT | 1067 |
| TACGGG | 609 |
| ATTCGT | 968 |
| ACACGT | 1725 |
| GCTTCG | 1148 |
| ACCCGC | 1395 |
| CGTATA | 738 |
| GTCACG | 1115 |
| TCGCAT | 737 |
| ACGGGC | 1160 |
| TCGCTT | 1476 |
| CGCATA | 484 |
| TGTCCG | 1311 |
| ACGACG | 271 |
| CGGTCC | 1022 |
| GATACG | 710 |
| TCGAAG | 963 |
| TCGGTG | 1210 |
| GGCGCT | 1428 |
| ATTTCG | 976 |
| GTTCGC | 494 |
| GCGACT | 688 |
| GTCGTC | 751 |
| CTCGCT | 1846 |
| CAACCG | 670 |
| TTTACG | 1103 |
| TACGTG | 1340 |
| GCGGCG | 760 |
| TGGCGG | 1796 |
| GCCGGA | 1350 |
| AGCGCG | 451 |
| TGCGAG | 1016 |
| CGTCGA | 212 |
| TCCGCC | 1944 |
| GGGTCG | 970 |
| ACGGCT | 1206 |
| GACCGC | 933 |
| CGGTAA | 592 |
| GAACGT | 1181 |
| TGCGTA | 799 |
| CGGGTA | 636 |
| TGGCGT | 1585 |
| CTCGTT | 1278 |
| CGCCTA | 702 |
| TAGCGG | 545 |
| TACGAG | 621 |
| GCGGAC | 799 |
| ATGCGC | 769 |
| ATCGAC | 502 |
| CTCGAT | 864 |
| TTCGTT | 1520 |
| CACGAG | 1480 |
| TCTCGA | 1389 |
| CAGCGG | 1971 |
| CCGATA | 432 |
| ATTCCG | 910 |
| ACGTGA | 1640 |
| GGCCGA | 1910 |
| GAGACG | 1877 |
| GTACGC | 354 |
| TATGCG | 603 |
| GTCGGT | 715 |
| CCCGGT | 1351 |
| CGTGAT | 1480 |
| AACTCG | 983 |
| CTTACG | 929 |
| TCGGAG | 1289 |
| TTCGAT | 796 |
| GCGTTG | 972 |
| GTCGCA | 604 |
| CGACGG | 295 |
| CCCGCA | 1751 |
| GCTCGG | 1346 |
| TCGCCC | 1538 |
| ACGACC | 651 |
| CGTGTT | 1985 |
| CGATCC | 649 |
| ACGCAA | 818 |
| AGCGCC | 1468 |
| CCGTAC | 531 |
| CGCTCA | 1184 |
| GGAACG | 1154 |
| CGGAGC | 1632 |
| AAGCGA | 1314 |
| AACGAA | 1232 |
| GTCGTA | 536 |
| GTGCGT | 1360 |
| TCGTCC | 1012 |
| CGTCAA | 780 |
| GCACGT | 1569 |
| AAACGC | 1216 |
| CCGCGG | 987 |
| CGTTGT | 1279 |
| CGGGCA | 1984 |
| CGCATC | 872 |
| CGACTG | 1026 |
| CGTTCA | 1163 |
| AGACGA | 1066 |
| CGCTGT | 1839 |
| GTTTCG | 1020 |
| TGCGGC | 1333 |
| ATCGGC | 671 |
| GCGACG | 328 |
| ACCTCG | 1653 |
| CGTCTG | 1855 |
| CCGTCA | 1225 |
| TGCACG | 1737 |
| GCGGGC | 1837 |
| CGTTGC | 1015 |
| CGACGT | 335 |
| CGCCGC | 886 |
| ATCACG | 1282 |
| ACTTCG | 1072 |
| CGACAG | 1221 |
| TACGTA | 1084 |
| GAACGG | 905 |
| CCGATC | 577 |
| TCGAGC | 773 |
| CGGACG | 451 |
| GGCGCG | 877 |
| ACCGGA | 857 |
| ACGGCG | 418 |
| TATCGA | 626 |
| ATTCGC | 566 |
| CGCAGA | 1412 |
| TTCGCC | 947 |
| ACGACT | 747 |
| ACGAAT | 1003 |
| ACGTAG | 965 |
| CACGGT | 1636 |
| ATCGTC | 763 |
| ACACGC | 1298 |
| AACCCG | 1203 |
| TACGCA | 649 |
| ACGCGA | 207 |
| CGCTAT | 530 |
| CGGAAC | 787 |
| ACCGAA | 941 |
| AAGGCG | 1204 |
| AGATCG | 1145 |
| GGGCGC | 1730 |
| GGCGAC | 1013 |
| CACGCA | 1659 |
| CGAATA | 700 |
| GCGAAC | 525 |
| AACGGA | 984 |
| TACGGT | 715 |
| CGTAGA | 824 |
| AGCGAT | 1161 |
| CCCGTA | 796 |
| CGGGTC | 1131 |
| GCGGTC | 707 |
| CCGCGT | 620 |
| CTCGCC | 1677 |
| AGCGTT | 1270 |
| TCGGCA | 1056 |
| TGTACG | 933 |
| ATACCG | 618 |
| TTCCGG | 1186 |
| AGAGCG | 1522 |
| GTGCGG | 1370 |
| GTCGAG | 744 |
| CGCTTT | 1526 |
| ACTCGT | 957 |
| GTTCGT | 836 |
| CGTTAT | 910 |
| CATGCG | 1096 |
| TCGGGT | 973 |
| TGCGTC | 1195 |
| TCCCGT | 1631 |
| GTCGTG | 1087 |
| CACGTC | 1540 |
| GACCGT | 940 |
| CGACTA | 353 |
| GTTCGG | 684 |
| CCGTAT | 807 |
| GCGGTA | 488 |
| TCCACG | 1775 |
| CGGGAC | 1501 |
| CTAACG | 695 |
| AAACGA | 1458 |
| CGCCAC | 1951 |
| AGCGGT | 930 |
| TTTTCG | 1405 |
| TCGCTA | 536 |
| GCGTAA | 549 |
| TGTCGG | 1125 |
| ACTGCG | 1241 |
| CCGCTC | 1549 |
| CGGTTG | 836 |
| TTCGAG | 1329 |
| CGCAAA | 913 |
| TTGCGG | 946 |
| TTTCGT | 1594 |
| GTACGT | 896 |
| GCGAGC | 937 |
| ATACGG | 699 |
| CCGTTT | 1776 |
| ACGGTG | 1663 |
| ACGAAG | 1140 |
| GCACGG | 1594 |
| TCCGGC | 1214 |
| ATCGAA | 788 |
| GATCCG | 846 |
| CTCCGG | 1797 |
| TGCCGC | 1683 |
| ATGCGA | 734 |
| GGCACG | 1737 |
| CCGCTA | 543 |
| TCGTCA | 985 |
| GGCGGC | 1783 |
| ACGCCC | 1697 |
| CGTAAA | 1045 |
| CATCGA | 844 |
| CGAATC | 712 |
| AACGCC | 893 |
| CGACCA | 766 |
| TCTACG | 746 |
| GCCCGT | 1458 |
| GCGGCA | 1219 |
| GGTACG | 510 |
| ACGACA | 888 |
| TTCGCA | 741 |
| CGATAA | 558 |
| CACGTA | 1097 |
| ACGGGG | 1910 |
| TCCGTC | 1531 |
| TTACGC | 553 |
| CGTCGG | 392 |
| ACCCGG | 1823 |
| CAGCGT | 1924 |
| ACGAGT | 780 |
| TAACGG | 616 |
| CCTACG | 720 |
| TGACGT | 1395 |
| TTCGGT | 991 |
| GTCGGG | 1295 |
| AGCGCA | 1074 |
| CGCATT | 973 |
| TCCGAC | 650 |
| CGATTG | 578 |
| TGCTCG | 1227 |
| AATCGT | 980 |
| ATCTCG | 1355 |
| TCGCGC | 422 |
| CGGAAT | 913 |
| CGGTAG | 574 |
| CGGCGA | 396 |
| CGCGAA | 184 |
| TAACGT | 1151 |
| TGTTCG | 1037 |
| GCGGGT | 1376 |
| GGCGTC | 1042 |
| TACCGC | 543 |
| CGACGC | 352 |
| GCGGAG | 1805 |
| CCGTGC | 1827 |
| ATCCCG | 1127 |
| ACGTCT | 1404 |
| ATGGCG | 1309 |
| ACGAGG | 1464 |
| TCGTGC | 1294 |
| CGTCGT | 344 |
| AGCGGG | 1740 |
| AATTCG | 821 |
| CGAAGA | 1198 |
| CCCGCG | 917 |
| ATCGGA | 688 |
| TGTCGT | 1210 |
| CGTATT | 1193 |
| TATACG | 681 |
| CGTCCA | 1346 |
| ACCGCC | 1385 |
| TCGCTC | 1342 |
| CTAGCG | 489 |
| AGCGAG | 1599 |
| CGCTCG | 449 |
| GGCGTA | 504 |
| TTGCGT | 1071 |
| CACGGC | 1725 |
| TTCGTA | 983 |
| TCGTAT | 894 |
| ACGCAT | 918 |
| CGACTC | 936 |
| GGGCGT | 1576 |
| CCGCGC | 907 |
| TCGTTT | 1930 |
| GACCGG | 946 |
| CCCGAC | 1387 |
| GATCGC | 870 |
| AAATCG | 1144 |
| AGTCCG | 788 |
| AACGAT | 861 |
| TCGAGA | 1461 |
| CGGGCG | 1234 |
| CACACG | 1946 |
| ATTCGA | 746 |
| CGGACT | 940 |
| CGCGGA | 482 |
| ACGCTT | 1103 |
| CGTTCG | 224 |
| TAGACG | 650 |
| TGCGGA | 1150 |
| ACACGA | 1022 |
| GCGTCC | 1314 |
| CGCCCG | 1158 |
| AAAGCG | 1296 |
| GCTCGA | 777 |
| CCGAGA | 1934 |
| CGTCAG | 1284 |
| AACGTT | 1676 |
| ACGAGC | 849 |
| TACGGA | 744 |
| GACGCC | 1152 |
| CCGTCG | 411 |
| CGACAC | 842 |
| TAGGCG | 632 |
| TCAACG | 811 |
| GCGCCC | 1896 |
| TCGCAC | 851 |
| CGGACC | 1054 |
| TTACCG | 767 |
| AGCGGC | 1325 |
| CGGCAA | 871 |
| CGTAGG | 725 |
| AGCACG | 1424 |
| CTATCG | 455 |
| CCCCGA | 1963 |
| CGAAAA | 1347 |
| ATCGGG | 824 |
| GGCGCA | 1317 |
| TCCCGA | 1673 |
| CACGCG | 683 |
| CGTTCT | 1458 |
| GCGAGT | 812 |
| TCGCCG | 426 |
| CGCTCT | 1732 |
| TCGGGA | 1711 |
| CGCAGG | 1787 |
| TTTCGC | 879 |
| CCGCCG | 1031 |
| TACCCG | 757 |
| TTCGTC | 828 |
| AGTACG | 583 |
| GCGACA | 940 |
| ACGGCA | 1177 |
| TTCACG | 1487 |
| TGACGC | 874 |
| GCTGCG | 1963 |
| ACGTAA | 1019 |
| CCGCAC | 1408 |
| GGCGGT | 1030 |
| CCAACG | 978 |
| TCCGCG | 476 |
| GAACGA | 877 |
| ACGGTA | 645 |
| CGGGCT | 1705 |
| CGTCTA | 628 |
| ATTCGG | 748 |
| CCGAAA | 1154 |
| GGCGAG | 1434 |
| AACCGT | 1020 |
| ATCGTG | 1336 |
| GTCGAA | 481 |
| AATCCG | 794 |
| GTGCGA | 776 |
| ACACGG | 1486 |
| CGGTGA | 1309 |
| TTCGGC | 869 |
| GCGGTG | 1816 |
| GCGAAG | 884 |
| TCGAAA | 981 |
| CTACGA | 568 |
| TGGCGA | 1177 |
| TGCGAA | 878 |
| GTACGG | 445 |
| CACGAC | 796 |
| CAGCGC | 1780 |
| CTGACG | 1282 |
| ATACGT | 1210 |
| ACGGAG | 1530 |
| CACGCT | 1588 |
| CGGTTC | 974 |
| GACGAT | 720 |
| GGTCCG | 808 |
| CGAATT | 911 |
| AATCGC | 952 |
| CTTGCG | 920 |
| CCCGTT | 1345 |
| GAATCG | 1139 |
| AACCGG | 728 |
| TAACGC | 519 |
| CCCGAT | 816 |
| AGGCGT | 1710 |
| TACGAA | 883 |
| TAGCGA | 526 |
| GCGCAT | 805 |
| TCGATT | 834 |
| CGTAGT | 727 |
| AGCGTA | 674 |
| GACGTT | 1193 |
| CGTCGC | 348 |
| GAAGCG | 1291 |
| ACTCGA | 806 |
| ACGTCC | 1187 |
| TGTCGC | 1164 |
| GCACGA | 953 |
| GCGCTT | 1017 |
| TCGGAA | 1039 |
| CGCAAG | 763 |
| CAGTCG | 1011 |
| GTTCGA | 975 |
| CGCGTG | 737 |
| ACCCGT | 1130 |
| CGGGAT | 1040 |
| CGATGA | 929 |
| TCGTCG | 229 |
| TTCGAC | 583 |
| CCGATT | 781 |
| ACGGGT | 891 |
| AGCGTC | 1205 |
| TTGCGC | 712 |
| CCGGAA | 1274 |
| CGTAAG | 748 |
| GTCTCG | 1514 |
| TACTCG | 860 |
| CGCCAT | 1318 |
| CACCGA | 1244 |
| TTTCCG | 1378 |
| GATCGT | 849 |
| GCATCG | 932 |
| CGAGGA | 1679 |
| CGATAG | 432 |
| TGACCG | 1175 |
| CCCGCT | 1988 |
| CGCCTT | 1673 |
| CGGTTA | 581 |
| TCCGCT | 1181 |
| GATTCG | 637 |
| GTCGGA | 712 |
| GCGAGG | 1438 |
| CATCGG | 987 |
| GTGGCG | 1963 |
| GTCCCG | 1397 |
| CAAACG | 1140 |
| GCGTCT | 1348 |
| CGGATG | 1100 |
| CGGGTT | 1208 |
| CGACCG | 255 |

We claim:

1. A method of designing a library of siRNA sequences, said method comprising collecting a set of siRNA sequences of at least 100 siRNAs that target at least 25 different genes, wherein said siRNA sequences comprise 18-30 bases, and at least 25% of the siRNA sequences have at positions 2-7 of an antisense sequence a hexamer sequence that is the reverse complement of a sequence selected from the group consisting of:
GCAGCG, ATATCG, CAATCG, TCGGAT, GTGACG, CCGCAT,
CACGAT, GACGCT, CGTCCG, CGAAGG, GTTGCG, GCCGTT,
ACGCGC, ACCGAC, TGTGCG, TCGTTA, TTTCGA, TAATCG,
GCGCCT, GCCGAT, TCGGTT, TACGAT, GTCCGC, AGCTCG,
TCGATG, TCACCG, TTCGGA, CAAGCG, CACGTT, AACGGC,
ATAGCG, GGTCGC, TCTCGC, AGTTCG, CGACCT, TGCCGG,
TTGGCG, GAGTCG, AGCCCG, CCGCTT, AACACG, ACGAGA,
CCACGA, AGCGGA, CGCTCC, CTTCGA, AGGGCG, ATCCGT,
TGCGCC, TCGCAA, TTCTCG, AGACGC, GCGATT, AGGCGA,
AGCGAA, CATCGT, GACCGA, CGTTCC, TTCCCG, CGGGCC,
GCGGAA, CTCTCG, CGATTA, CGTCAC, CGCAGT, CATTCG,
TACGTT, CGAGAA, CGTACA, CCATCG, ACCGCG, GCCGCT,
GATCGG, GAAACG, ACGTGC, CTCGGA, TAAGCG, TCGACC,
TATCGT, CGCGGG, AGTCGT, GGACCG, CGCACA, CTGGCG,
CGGATA, CGTAGC, TCGGCC, GCGTCG, ACCGGC, CGGCAG,
TACGCC, ACCACG, ACGCTA, TCGCTG, CGCGCA, GTATCG,
CGTGAA, GACGCG, GCCCGA, AACGTA, AGTCGG, GCGGGA,
AAGCGT, CCGAGT, CGAAAG, CGAGTG, ACTACG, GCGCCG,
AATCGA, TTCGAA, TTGCGA, CCGACA, GCGCAC, TCGTTC,
TAACGA, CGACTT, ACGCTC, CGCGGT, ACGTAT, GCAACG,
ATAACG, TTACGG, AACGTC, TCCGTG, CAACGA, CGACAT,
CTGCGA, TGTCGA, TCCGGG, ATCCGG, CGCGAG, CGGCGG,
CGATTC, GCGAAA, CTCGAA, GTACGA, GAGCGC, CGGTAC,
CCGAAG, CTACGG, GACGAC, CCGGTG, AGTCGC, CGTCTT,
TCGTGG, CGTAAC, ACGGAA, AACCGA, CGCGTC, CCGGGT,
TCGTAC, AAGCCG, GGCGAA, GGGCGA, ACGATT, GGACGC,
CGCAAC, TCCGCA, TGACGG, CGGTGT, AGACCG, GCGTGC,
CCGGAG, GGTCGT, TCCGGT, CGGTCA, AATCGG, GCCGCG,
ACCGCT, CGCGTA, TATCGC, ACATCG, TACCGG, CGGCGT,
TGCCGT, GTAGCG, GACGGC, ATCCGC, TCTCCG, CGTTAA,
GGCTCG, ACCGAT, ACGCCT, CGATGG, CACCGG, CGACCC,
CGGATC, GCGCGC, GCCGAC, CGGCCA, ATTGCG, ACCGTT,
CGATAC, CATCGC, AACGCT, CGCTAA, ATGACG, CGTCCT,
ACAGCG, CGAAGT, GTCCGT, AGCGTG, TCGCGG, CGCAGC,
TCCGAG, GGCGGA, GCGAGA, GACACG, CCTCGA, CGAACA,
AAGTCG, CCGTCC, TTACGT, CGAGGG, GGTTCG, AACGCG,
TCCGTA, CTTCGG, CCGGTA, TCGCGT, CTCGTG, CGGCTC,
CGATGT, CACCGT, GACGTC, CGGTAT, TTCGTG, TACCGT,
ACAACG, GTAACG, CGTTTG, GCGTAT, CGATCA, GCGCTC,
TTTCGG, CCGTAA, CTACGT, TCGTGT, ACGCAC, TGGACG,
CGAGGT, CCGAGC, AACGAC, AAGCGC, TCGATC, TCGCCA,
ATACGA, CGAGCA, GTCCGG, CGGTTT, ACGAAA, GCGTTT,
CATCCG, TCGATA, CGCACG, GCGCTA, TTCGGG, GCCGGC,
CGCGGC, ACGTCG, GCCGTC, CGAGAG, TATCCG, CCGGCA,
CGTACG, CGTCAT, GATCGA, ACGCCG, TCCCAG, GCTACG,
CGGCTA, GAGCGT, ACGGGA, GGTCGG, GACGTA, ACCCGA,
GCGTCA, CGATTT, TTAACG, TCGAAC, AACGTG, CTTTCG,
CCGACG, TGCGAC, ACGGCC, TACGTC, CGATAT, CGAAAC,
TGGCGC, GGCCGC, GGACGT, GCGATC, TGCGCG, CGCACT,
CAACGG, ACCGGG, TACACG, GCGCCA, CGGTGC, GCGTGT,
ACTCGA, TCGGTC, CGCGCG, CGTGAG, ATCGCT, GGGACG,
CGGCGC, CGCGAC, TCGTAA, TCGGTA, AGCCGT, GACGGT,
AACGGG, GCCGTA, CCGGTC, ATGTCG, CTACGC, TAGCGT,
CGAGTA, ACTCCG, TCACGG, GACGCA, GCGCGT, CGTACT,
CCGAAC, CGAAGC, CGGAGA, GTCGCC, GCGCAG, CTTCGT,
CGTCCC, ATGCCG, ATCCGA, ACGCTG, CTCGAG, CGCTTG,
GATGCG, CCGGAC, CAACGT, CGCTGA, CGGTCG, GTCGTT,
GCGATA, GACGAG, CGTGTA, GCTAGC, TCTCGG, ACGGAT,
CGCGCT, TGAACG, GAGCGG, CGGCCG, CTCGGT, GCCGGT,
TCGTTG, TAGCGC, ACGATG, ACACCG, ACGGTT, TACGAC,
ACGTTA, AGTGCG, CGTTGA, CGCAAT, CGCTAG, CGCCGA,
CAGACG, GGACGG, CTCGCA, GCCGCA, TGCCGA, GTTACG,
CGATGC, CACCGC, CCGTTG, TTCCGT, TCGGGC, GCGTAC,
AAACCG, CGTTAG, CGTAAT, CGAACG, CTCGTA, TTAGCG,
ACGTTC, CTGCGT, TCGACG, TACGGC, ACCGTG, GTCGAT,
ATCGCG, CGAGTC, CGGAAA, GCGCGG, CGTGCA, CGGCAC,
TCACGT, ACTCGC, TCCCGC, TTATCG, TCCTCG, ACGATC,
AACGCA, ACGCGT, GCTCCG, CGCTTA, TCTTCG, GTGTCG,
CGATCG, ACCGTA, CACCCG, AACGGT, GACGGG, CGCGAT,
CACGGA, GGCCGT, TAAACG, GACGTG, TTACGA, CGTATG,
CGTGTC, CCTCGT, CGCACC, TATCGG, AATGCG, TCTCGT,
GCGCTG, GTCCGA, CGAGCG, GTGCCG, CGCGTT, CGCATG,
CTACCG, CGTTTA, CGAACT, ATCGCC, ACCGTC, TCGGAC,
CCTTCG, AGACGT, AGCCGC, CGCCAA, TGGTCG, CGAGAC,
CGTACC, CGGGAA, GCGGCC, CTCGTC, CCGACT, TCGGCG,
GAACCG, ACGTCA, CCCGGA, AGGACG, CATACG, TCGACT,
CTTCGC, GTCGCT, TCCGGA, GGTCGA, CGGATT, ACGCCA,
TGCGCT, CCGGCG, TACGCG, GTCGCG, CAGCGA, CACGAA,
TTTGCG, ACCGGT, TACGCT, CAACGC, CGGCAT, CCGCAA,
CGCGCC, CGTGAC, GCGTTC, TCGTGA, TTGACG, CGACGA,
ACGTAC, TGACGA, TATTCG, CGAAAT, GCTCGC, TTCCGC,
CGGCTT, TCGGCT, ACGCGG, ACCGAG, ACGCAG, TGCGAT,
GGTGCG, GCGTTA, TAGCCG, ATCGAT, GCACCG, GCGATG,
CCGTGA, CGTTTC, TACCGA, CTTCCG, AAGCGG, GCGGAT,
CTGCGC, CTCGAC, ACGATA, CCGGCT, AACGAG, TGAGCG,
TGCGTT, CGCTTC, ATCGTT, GCGACC, CGGTCT, CCGAAT,
CCGTAG, CCGCGA, CCCGAA, TAGTCG, ATTACG, CACTCG,
TCGCGA, TCCGAA, AGACGG, ACCGCA, GCGGTT, TGATCG,
TCACGC, TCGAAT, TCGTAG, GAACGC, CTCGCG, AGCCGA,
CGAGTT, CGCTAC, GACGAA, GAGCGA, CGAATG, ATGCGT,
ATCGTA, TTCGCG, CGAGAT, AGAACG, GCGCAA, CCGTTC,
TCGAGG, GGCGCC, GTCGGC, TCACGA, CCTCGC, ACTCGG,
CGCCGG, CGAACC, GCGGCT, CGGACA, GGACGA, TAACCG,
CGTTAC, CGTTGG, AGCGCT, GCGTGA, AATACG, GTTCCG,
CGTGCG, CCGTTA, CGATCT, TCAGCG, GTCGAC, TCCGTT,
GTGCGC, CGGAGT, CGACAA, ACGGAC, CCGGAT, GCGCGA,
GCCGAA, TTCCGA, CGGAAG, AACCGC, CGGGTG, GCGAAT,
AGGTCG, GCACGC, GCGTAG, TCGTCT, CCGACC, CGAGCT,
TGCGGG, TTGCCG, ACGTTG, ATCGCA, TCATCG, CCGGTT,
CCGATG, TCGCCT, GACTCG, TCCGAT, AAGACG, TTGTCG,
AAACGG, GTACCG, ATCGGT, GGCGTT, ATACGC, CGTATC,
ACGAAC, TCTGCG, ACGGTC, GGCGAT, GACGGA, CACGGG,
CTGTCG, CGAGCC, AGCGAC, AGGCGC, GACCCG, GGATCG,
CGGGGT, CGCCGT, TCGACA, CGTGCT, CTCCGA, TGCGCA,
CGCCAG, TCGGGG, GCTCGT, ATGCGG, ATCGAG, TCGAGT,
GGAGCG, TGCGGT, TTCGCT, TACGGG, ATTCGT, ACACGT,
GCTTCG, ACCCGC, CGTATA, GTCACG, TCGCAT, ACGGGC,
TCGCTT, CGCATA, TGTCCG, ACGACG, CGGTCC, GATACG,
TCGAAG, TCGGTG, GGCGCT, ATTTCG, GTTCGC, GCGACT,
GTCGTC, CTCGCT, CAACCG, TTTACG, TACGTG, GCGGCG,
TGGCGG, GCCGGA, AGCGCG, TGCGAG, CGTCGA, TCCGCC,
GGGTCG, ACGGCT, GACCGC, CGGTAA, GAACGT, TGCGTA,
CGGGTA, TGGCGT, CTCGTT, CGCCTA, TAGCGG, TACGAG,
GCGGAC, ATGCGC, ATCGAC, CTCGAT, TTCGTT, CACGAG,
TCTCGA, CAGCGG, CCGATA, ATTCCG, ACGTGA, GGCCGA,
GAGACG, GTACGC, TATGCG, GTCGGT, CCCGGT, CGTGAT,
AACTCG, CTTACG, TCGGAG, TTCGAT, GCGTTG, GTCGCA,
CGACGG, CCCGCA, GCTCGG, TCGCCC, ACGACC, CGTGTT,
CGATCC, ACGCAA, AGCGCC, CCGTAC, CGCTCA, GGAACG,
CGGAGC, AAGCGA, AACGAA, GTCGTA, GTGCGT, TCGTCC,
CGTCAA, GCACGT, AAACGC, CCGCGG, CGTTGT, CGGGCA,
CGCATC, CGACTG, CGTTCA, AGACGA, CGCTGT, GTTTCG,
TGCGGC, ATCGGC, GCGACG, ACCTCG, CGTCTG, CCGTCA,
TGCACG, GCGGGC, CGTTGC, CGACGT, CGCCGC, ATCACG,
ACTTCG, CGACAG, TACGTA, GAACGG, CCGATC, TCGAGC,
CGGACG, GGCGCG, ACCGGA, ACGGCG, TATCGA, ATTCGC,
CGCAGA, TTCGCC, ACGACT, ACGAAT, ACGTAG, CACGGT,
ATCGTC, ACACGC, AACCCG, TACGCA, ACGCGA, CGCTAT,
CGGAAC, ACCGAA, AAGGCG, AGATCG, GGGCGC, GGCGAC,
CACGCA, CGAATA, GCGAAC, AACGGA, TACGGT, CGTAGA,
AGCGAT, CCCGTA, CGGGTC, GCGGTC, CCGCGT, CTCGCC,
AGCGTT, TCGGCA, TGTACG, ATACCG, TTCCGG, AGAGCG,
GTGCGG, GTCGAG, CGCTTT, ACTCGT, GTTCGT, CGTTAT,
CATGCG, TCGGGT, TGCGTC, TCCCGT, GTCGTG, CACGTC,
GACCGT, CGACTA, GTTCGG, CCGTAT, GCGGTA, TCCACG,
CGGGAC, CTAACG, AAACGA, CGCCAC, AGCGGT, TTTTCG,
TCGCTA, GCGTAA, TGTCGG, ACTGCG, CCGCTC, CGGTTG,
TTCGAG, CGCAAA, TTGCGG, TTTCGT, GTACGT, GCGAGC,
ATACGG, CCGTTT, ACGGTG, ACGAAG, GCACGG, TCCGGC,
ATCGAA, GATCCG, CTCCGG, TGCCGC, ATGCGA, GGCACG,
CCGCTA, TCGTCA, GGCGGC, ACGCCC, CGTAAA, CATCGA,
CGAATC, AACGCC, CGACCA, TCTACG, GCCCGT, GCGGCA,
GGTACG, ACGACA, TTCGCA, CGATAA, CACGTA, ACGGGG,
TCCGTC, TTACGC, CGTCGG, ACCCGG, CAGCGT, ACGAGT,
TAACGG, CCTACG, TGACGT, TTCGGT, GTCGGG, AGCGCA,
CGCATT, TCCGAC, CGATTG, TGCTCG, AATCGT, ATCTCG,
TCGCGC, CGGAAT, CGGTAG, CGGCGA, CGCGAA, TAACGT,
TGTTCG, GCGGGT, GGCGTC, TACCGC, CGACGC, GCGGAG,
CCGTGC, ATCCCG, ACGTCT, ATGGCG, ACGAGG, TCGTGC,
CGTCGT, AGCGGG, AATTCG, CGAAGA, CCCGCG, ATCGGA,
TGTCGT, CGTATT, TATACG, CGTCCA, ACCGCC, TCGCTC,
CTAGCG, AGCGAG, CGCTCG, GGCGTA, TTGCGT, CACGGC,
TTCGTA, TCGTAT, ACGCAT, CGACTC, GGGCGT, CCGCGC,
TCGTTT, GACCGG, CCCGAC, GATCGC, AAATCG, AGTCCG,
AACGAT, TCGAGA, CGGGCG, CACACG, ATTCGA, CGGACT,
CGCGGA, ACGCTT, CGTTCG, TAGACG, TGCGGA, ACACGA,
GCGTCC, CGCCCG, AAAGCG, GCTCGA, CCGAGA, CGTCAG,
AACGTT, ACGAGC, TACGGA, GACGCC, CCGTCG, CGACAC,
TAGGCG, TCAACG, GCGCCC, TCGCAC, CGGACC, TTACCG,
AGCGGC, CGGCAA, CGTAGG, AGCACG, CTATCG, CCCCGA,
CGAAAA, ATCGGG, GGCGCA, TCCCGA, CACGCG, CGTTCT,
GCGAGT, TCGCCG, CGCTCT, TCGGGA, CGCAGG, TTTCGC,
CCGCCG, TACCCG, TTCGTC, AGTACG, GCGACA, ACGGCA,
TTCACG, TGACGC, GCTGCG, ACGTAA, CCGCAC, GGCGGT,
CCAACG, TCCGCG, GAACGA, ACGGTA, CGGGCT, CGTCTA,
ATTCGG, CCGAAA, GGCGAG, AACCGT, ATCGTG, GTCGAA,
AATCCG, GTGCGA, ACACGG, CGGTGA, TTCGGC, GCGGTG,
GCGAAG, TCGAAA, CTACGA, TGGCGA, TGCGAA, GTACGG,
CACGAC, CAGCGC, CTGACG, ATACGT, ACGGAG, CACGCT,
CGGTTC, GACGAT, GGTCCG, CGAATT, AATCGC, CTTGCG,
CCCGTT, GAATCG, AACCGG, TAACGC, CCCGAT, AGGCGT,
TACGAA, TAGCGA, GCGCAT, TCGATT, CGTAGT, AGCGTA,
GACGTT, CGTCGC, GAAGCG, ACTCGA, ACGTCC, TGTCGC,
GCACGA, GCGCTT, TCGGAA, CGCAAG, CAGTCG, GTTCGA,
CGCGTG, ACCCGT, CGGGAT, CGATGA, TCGTCG, TTCGAC,
CCGATT, ACGGGT, AGCGTC, TTGCGC, CCGGAA, CGTAAG,
GTCTCG, TACTCG, CGCCAT, CACCGA, TTTCCG, GATCGT,
GCATCG, CGAGGA, CGATAG, TGACCG, CCCGCT, CGCCTT,
CGGTTA, TCCGCT, GATTCG, GTCGGA, GCGAGG, CATCGG,
GTGGCG, GTCCCG, CAAACG, GCGTCT, CGGATG, CGGGTT,
and CGACCG.

2. The method according to claim 1, wherein said set of siRNA sequences comprises sequences of at least 200 siRNAs.

3. The method according to claim 1, wherein said set of siRNA sequences comprises sequences of at least 500 siRNAs.

4. The method according to claim 1, wherein said set of siRNA sequences comprises sequences of at least 1000 siRNAs.

5. The method according to claim 1, wherein said set of siRNA targets at least 50 different genes.

6. The method according to claim 1, wherein said set of siRNA targets at least 100 different genes.

7. The method according to claim 1, wherein said set of siRNA sequences that target at least 25,000 different genes wherein at least 25% of the siRNA sequences have at positions 2-7 of an antisense sequence a hexamer sequence that is the reverse complement of a sequence selected from the group consisting of
GCAGCG, ATATCG, CAATCG, TCGGAT, GTGACG, CCGCAT,
CACGAT, GACGCT, CGTCCG, CGAAGG, GTTGCG, GCCGTT,
ACGCGC, ACCGAC, TGTGCG, TCGTTA, TTTCGA, TAATCG,
GCGCCT, GCCGAT, TCGGTT, TACGAT, GTCCGC, AGCTCG,
TCGATG, TCACCG, TTCGGA, CAAGCG, CACGTT, AACGGC,
ATAGCG, GGTCGC, TCTCGC, AGTTCG, CGACCT, TGCCGG,
TTGGCG, GAGTCG, AGCCCG, CCGCTT, AACACG, ACGAGA,
CCACGA, AGCGGA, CGCTCC, CTTCGA, AGGGCG, ATCCGT,
TGCGCC, TCGCAA, TTCTCG, AGACGC, GCGATT, AGGCGA,
AGCGAA, CATCGT, GACCGA, CGTTCC, TTCCCG, CGGGCC,
GCGGAA, CTCTCG, CGATTA, CGTCAC, CGCAGT, CATTCG,
TACGTT, CGAGAA, CGTACA, CCATCG, ACCGCG, GCCGCT,
GATCGG, GAAACG, ACGTGC, CTCGGA, TAAGCG, TCGACC,
TATCGT, CGCGGG, AGTCGT, GGACCG, CGCACA, CTGGCG,
CGGATA, CGTAGC, TCGGCC, GCGTCG, ACCGGC, CGGCAG,
TACGCC, ACCACG, ACGCTA, TCGCTG, CGCGCA, GTATCG,
CGTGAA, GACGCG, GCCCGA, AACGTA, AGTCGG, GCGGGA,
AAGCGT, CCGACT, CGAAAG, CGAGTG, ACTACG, GCGCCG,
AATCGA, TTCGAA, TTGCGA, CCGACA, GCGCAC, TCGTTC,
TAACGA, CGACTT, ACGCTC, CGCGGT, ACGTAT, GCAACG,
ATAACG, TTACGG, AACGTC, TCCGTG, CAACGA, CGACAT,
CTGCGA, TGTCGA, TCCGGG, ATCCGG, CGCGAG, CGGCGG,
CGATTC, GCGAAA, CTCGAA, GTACGA, GAGCGC, CGGTAC,
CCGAAG, CTACGG, GACGAC, CCGGTG, AGTCGC, CGTCTT,
TCGTGG, CGTAAC, ACGGAA, AACCGA, CGCGTC, CCGGGT,
TCGTAC, AAGCCG, GGCGAA, GGGCGA, ACGATT, GGACGC,
CGCAAC, TCCGCA, TGACGG, CGGTGT, AGACCG, GCGTGC,
CCGGAG, GGTCGT, TCCGGT, CGGTCA, AATCGG, GCCGCG,
ACCGCT, CGCGTA, TATCGC, ACATCG, TACCGG, CGGCGT,
TGCCGT, GTAGCG, GACGGC, ATCCGC, TCTCCG, CGTTAA,
GGCTCG, ACCGAT, ACGCCT, CGATGG, CACCGG, CGACCC,
CGGATC, GCGCGC, GCCGAC, CGGCCA, ATTGCG, ACCGTT,
CGATAC, CATCGC, AACGCT, CGCTAA, ATGACG, CGTCCT,
ACAGCG, CGAAGT, GTCCGT, AGCGTG, TCGCGG, CGCAGC,
TCCGAG, GGCGGA, GCGAGA, GACACG, CCTCGA, CGAACA,
AAGTCG, CCGTCC, TTACGT, CGAGGG, GGTTCG, AACGCG,
TCCGTA, CTTCGG, CCGGTA, TCGCGT, CTCGTG, CGGCTC,
CGATGT, CACCGT, GACGTC, CGGTAT, TTCGTG, TACCGT,
ACAACG, GTAACG, CGTTTG, GCGTAT, CGATCA, GCGCTC,
TTTCGG, CCGTAA, CTACGT, TCGTGT, ACGCAC, TGGACG,
CGAGGT, CCGAGC, AACGAC, AAGCGC, TCGATC, TCGCCA,
ATACGA, CGAGCA, GTCCGG, CGGTTT, ACGAAA, GCGTTT,
CATCCG, TCGATA, CGCACG, GCGCTA, TTCGGG, GCCGGC,
CGCGGC, ACGTCG, GCCGTC, CGAGAG, TATCCG, CCGGCA,
CGTACG, CGTCAT, GATCGA, ACGCCG, TCGCAG, GCTACG,
CGGCTA, GAGCGT, ACGGGA, GGTCGG, GACGTA, ACCCGA,
GCGTCA, CGATTT, TTAACG, TCGAAC, AACGTG, CTTTCG,
CCGACG, TGCGAC, ACGGCC, TACGTC, CGATAT, CGAAAC,
TGGCGC, GGCCGC, GGACGT, GCGATC, TGCGCG, CGCACT,
CAACGG, ACCGGG, TACACG, GCGCCA, CGGTGC, GCGTGT,
AGTCGA, TCGGTC, CGCGCG, CGTGAG, ATCGCT, GGGACG,
CGGCGC, CGCGAC, TCGTAA, TCGGTA, AGCCGT, GACGGT,
AACGGG, GCCGTA, CCGGTC, ATGTCG, CTACGC, TAGCGT,
CGAGTA, ACTCCG, TCACGG, GACGCA, GCGCGT, CGTACT,
CCGAAC, CGAAGC, CGGAGA, GTCGCC, GCGCAG, CTTCGT,
CGTCCC, ATGCCG, ATCCGA, ACGCTG, CTCGAG, CGCTTG,
GATGCG, CCGGAC, CAACGT, CGCTGA, CGGTCG, GTCGTT,
GCGATA, GACGAG, CGTGTA, GCTAGC, TCTCGG, ACGGAT,
CGCGCT, TGAACG, GAGCGG, CGGCCG, CTCGGT, GCCGGT,
TCGTTG, TAGCGC, ACGATG, ACACCG, ACGGTT, TACGAC,
ACGTTA, AGTGCG, CGTTGA, CGCAAT, CGCTAG, CGCCGA,
CAGACG, GGACGG, CTCGCA, GCCGCA, TGCCGA, GTTACG,
CGATGC, CACCGC, CCGTTG, TTCCGT, TCGGGC, GCGTAC,
AAACCG, CGTTAG, CGTAAT, CGAACG, CTCGTA, TTAGCG,
ACGTTC, CTGCGT, TCGACG, TACGGC, ACCGTG, GTCGAT,
ATCGCG, CGAGTC, CGGAAA, GCGCGG, CGTGCA, CGGCAC,
TCACGT, ACTCGC, TCCCGC, TTATCG, TCCTCG, ACGATC,
AACGCA, ACGCGT, GCTCCG, CGCTTA, TCTTCG, GTGTCG,
CGATCG, ACCGTA, CACCCG, AACGGT, GACGGG, CGCGAT,
CACGGA, GGCCGT, TAAACG, GACGTG, TTACGA, CGTATG,
CGTGTC, CCTCGT, CGCACC, TATCGG, AATGCG, TCTCGT,
GCGCTG, GTCCGA, CGAGCG, GTGCCG, CGCGTT, CGCATG,
CTACCG, CGTTTA, CGAACT, ATCGCC, ACCGTC, TCGGAC,
CCTTCG, AGACGT, AGCCGC, CGCCAA, TGGTCG, CGAGAC,
CGTACC, CGGGAA, GCGGCC, CTCGTC, CCGACT, TCGGCG,
GAACCG, ACGTCA, CCCGGA, AGGACG, CATACG, TCGACT,
CTTCGC, GTCGCT, TCCGGA, GGTCGA, CGGATT, ACGCCA,
TGCGCT, CCGGCG, TACGCG, GTCGCG, CAGCGA, CACGAA,
TTTGCG, ACCGGT, TACGCT, CAACGC, CGGCAT, CCGCAA,
CGCGCC, CGTGAC, GCGTTC, TCGTGA, TTGACG, CGACGA,
ACGTAC, TGACGA, TATTCG, CGAAAT, GCTCGC, TTCCGC,
CGGCTT, TCGGCT, ACGCGG, ACCGAG, ACGCAG, TGCGAT,
GGTGCG, GCGTTA, TAGCCG, ATCGAT, GCACCG, GCGATG,
CCGTGA, CGTTTC, TACCGA, CTTCCG, AAGCGG, GCGGAT,
CTGCGC, CTCGAC, ACGATA, CCGGCT, AACGAG, TGAGCG,
TGCGTT, CGCTTC, ATCGTT, GCGACC, CGGTCT, CCGAAT,
CCGTAG, CCGCGA, CCCGAA, TAGTCG, ATTACG, CACTCG,
TCGCGA, TCCGAA, AGACGG, ACCGCA, GCGGTT, TGATCG,
TCACGC, TCGAAT, TCGTAG, GAACGC, CTCGCG, AGCCGA,
CGAGTT, CGCTAC, GACGAA, GAGCGA, CGAATG, ATGCGT,
ATCGTA, TTCGCG, CGAGAT, AGAACG, GCGCAA, CCGTTC,
TCGAGG, GGCGCC, GTCGGC, TCACGA, CCTCGC, ACTCGG,
CGCCGG, CGAACC, GCGGCT, CGGACA, GGACGA, TAACCG,
CGTTAC, CGTTGG, AGCGCT, GCGTGA, AATACG, GTTCCG,
CGTGCG, CCGTTA, CGATCT, TCAGCG, GTCGAC, TCCGTT,
GTGCGC, CGGAGT, CGACAA, ACGGAC, CCGGAT, GCGCGA,
GCCGAA, TTCCGA, CGGAAG, AACCGC, CGGGTG, GCGAAT,
AGGTCG, GCACGC, GCGTAG, TCGTCT, CCGACC, CGAGCT,
TGCGGG, TTGCCG, ACGTTG, ATCGCA, TCATCG, CCGGTT,
CCGATG, TCGCCT, GACTCG, TCCGAT, AAGACG, TTGTCG,
AAACGG, GTACCG, ATCGGT, GGCGTT, ATACGC, CGTATC,
ACGAAC, TCTGCG, ACGGTC, GGCGAT, GACGGA, CACGGG,
CTGTCG, CGAGCC, AGCGAC, AGGCGC, GACCCG, GGATCG,
CGGGGT, CGCCGT, TCGACA, CGTGCT, CTCCGA, TGCGCA,
CGCCAG, TCGGGG, GCTCGT, ATGCGG, ATCGAG, TCGAGT,
GGAGCG, TGCGGT, TTCGCT, TACGGG, ATTCGT, ACACGT,
GCTTCG, ACCCGC, CGTATA, GTCACG, TCGCAT, ACGGGC,
TCGCTT, CGCATA, TGTCCG, ACGACG, CGGTCC, GATACG,
TCGAAG, TCGGTG, GGCGCT, ATTTCG, GTTCGC, GCGACT,
GTCGTC, CTCGCT, CAACCG, TTTACG, TACGTG, GCGGCG,
TGGCGG, GCCGGA, AGCGCG, TGCGAG, CGTCGA, TCCGCC,
GGGTCG, ACGGCT, GACCGC, CGGTAA, GAACGT, TGCGTA,
CGGGTA, TGGCGT, CTCGTT, CGCCTA, TAGCGG, TACGAG,
GCGGAC, ATGCGC, ATCGAC, CTCGAT, TTCGTT, CACGAG,
TCTCGA, CAGCGG, CCGATA, ATTCCG, ACGTGA, GGCCGA,
GAGACG, GTACGC, TATGCG, GTCGGT, CCCGGT, CGTGAT,
AACTCG, CTTACG, TCGGAG, TTCGAT, GCGTTG, GTCGCA,
CGACGG, CCCGCA, GCTCGG, TCGCCC, ACGACC, CGTGTT,
CGATCC, ACGCAA, AGCGCC, CCGTAC, CGCTCA, GGAACG,
CGGAGC, AAGCGA, AACGAA, GTCGTA, GTGCGT, TCGTCC,
CGTCAA, GCACGT, AAACGC, CCGCGG, CGTTGT, CGGGCA,
CGCATC, CGACTG, CGTTCA, AGACGA, CGCTGT, GTTTCG,
TGCGGC, ATCGGC, GCGACG, ACCTCG, CGTCTG, CCGTCA,
TGCACG, GCGGGC, CGTTGC, CGACGT, CGCCGC, ATCACG,
ACTTCG, CGACAG, TACGTA, GAACGG, CCGATC, TCGAGC,
CGGACG, GGCGCG, ACCGGA, ACGGCG, TATCGA, ATTCGC,
CGCAGA, TTCGCC, ACGACT, ACGAAT, ACGTAG, CACGGT,
ATCGTC, ACACGC, AACCCG, TACGCA, ACGCGA, CGCTAT,
CGGAAC, ACCGAA, AAGGCG, AGATCG, GGGCGC, GGCGAC,
CACGCA, CGAATA, GCGAAC, AACGGA, TACGGT, CGTAGA,
AGCGAT, CCCGTA, CGGGTC, GCGGTC, CCGCGT, CTCGCC,
AGCGTT, TCGGCA, TGTACG, ATACCG, TTCCGG, AGAGCG,
GTGCGG, GTCGAG, CGCTTT, ACTCGT, GTTCGT, CGTTAT,
CATGCG, TCGGGT, TGCGTC, TCCCGT, GTCGTG, CACGTC,
GACCGT, CGACTA, GTTCGG, CCGTAT, GCGGTA, TCCACG,
CGGGAC, CTAACG, AAACGA, CGCCAC, AGCGGT, TTTTCG,
TCGCTA, GCGTAA, TGTCGG, ACTGCG, CCGCTC, CGGTTG,
TTCGAG, CGCAAA, TTGCGG, TTTCGT, GTACGT, GCGAGC,
ATACGG, CCGTTT, ACGGTG, ACGAAG, GCACGG, TCCGGC,
ATCGAA, GATCCG, CTCCGG, TGCCGC, ATGCGA, GGCACG,
CCGCTA, TCGTCA, GGCGGC, ACGCCC, CGTAAA, CATCGA,
CGAATC, AACGCC, CGACCA, TCTACG, GCCCGT, GCGGCA,
GGTACG, ACGACA, TTCGCA, CGATAA, CACGTA, ACGGGG,
TCCGTC, TTACGC, CGTCGG, ACCCGG, CAGCGT, ACGAGT,
TAACGG, CCTACG, TGACGT, TTCGGT, GTCGGG, AGCGCA,
CGCATT, TCCGAC, CGATTG, TGCTCG, AATCGT, ATCTCG,
TCGCGC, CGGAAT, CGGTAG, CGGCGA, CGCGAA, TAACGT,
TGTTCG, GCGGGT, GGCGTC, TACCGC, CGACGC, GCGGAG,
CCGTGC, ATCCCG, ACGTCT, ATGGCG, ACGAGG, TCGTGC,
CGTCGT, AGCGGG, AATTCG, CGAAGA, CCCGCG, ATCGGA,
TGTCGT, CGTATT, TATACG, CGTCCA, ACCGCC, TCGCTC,
CTAGCG, AGCGAG, CGCTCG, GGCGTA, TTGCGT, CACGGC,
TTCGTA, TCGTAT, ACGCAT, CGACTC, GGGCGT, CCGCGC,
TCGTTT, GACCGG, CCCGAC, GATCGC, AAATCG, AGTCCG,
AACGAT, TCGAGA, CGGGCG, CACACG, ATTCGA, CGGACT,
CGCGGA, ACGCTT, CGTTCG, TAGACG, TGCGGA, ACACGA,
GCGTCC, CGCCCG, AAAGCG, GCTCGA, CCGAGA, CCTCAG,
AACGTT, ACGAGC, TACGGA, GACGCC, CCGTCG, CGACAC,
TAGGCG, TCAACG, GCGCCC, TCGCAC, CGGACC, TTACCG,
AGCGGC, CGGCAA, CGTAGG, AGCACG, CTATCG, CCCCGA,
CGAAAA, ATCGGG, GGCGCA, TCCCGA, CACGCG, CGTTCT,
GCGAGT, TCGCCG, CGCTCT, TCGGGA, CGCAGG, TTTCGC,
CCGCCG, TACCCG, TTCGTC, AGTACG, GCGACA, ACGGCA,
TTCACG, TGACGC, GCTGCG, ACGTAA, CCGCAC, GGCGGT,
CCAACG, TCCGCG, GAACGA, ACGGTA, CGGGCT, CGTCTA,
ATTCGG, CCGAAA, GGCGAG, AACCGT, ATCGTG, GTCGAA,
AATCCG, GTGCGA, ACACGG, CGGTGA, TTCGGC, GCGGTG,
GCGAAG, TCGAAA, CTACGA, TGGCGA, TGCGAA, GTACGG,
CACGAC, CAGCGC, CTGACG, ATACGT, ACGGAG, CACGCT,
CGGTTC, GACGAT, GGTCCG, CGAATT, AATCGC, CTTGCG,
CCCGTT, GAATCG, AACCGG, TAACGC, CCCGAT, AGGCGT,
TACGAA, TAGCGA, GCGCAT, TCGATT, CGTAGT, AGCGTA,
GACGTT, CGTCGC, GAAGCG, ACTCGA, ACGTCC, TGTCGC,
GCACGA, GCGCTT, TCGGAA, CGCAAG, CAGTCG, GTTCGA,
CGCGTG, ACCCGT, CGGGAT, CGATGA, TCGTCG, TTCGAC,
CCGATT, ACGGGT, AGCGTC, TTGCGC, CCGGAA, CGTAAG,
GTCTCG, TACTCG, CGCCAT, CACCGA, TTTCCG, GATCGT,
GCATCG, CGAGGA, CGATAG, TGACCG, CCCGCT, CGCCTT,
CGGTTA, TCCGCT, GATTCG, GTCGGA, GCGAGG, CATCGG,
GTGGCG, GTCCCG, CAAACG, GCGTCT, CGGATG, CGGGTT,
and CGACCG.

8. The method according to claim 1, wherein at least 50% of the siRNA sequences have a hexamer sequence at positions 2-7 of said antisense sequence that is the reverse complement of a sequence selected from group consisting of
CCAGCG, ATATCG, CAATCG, TCGGAT, GTGACG, CCGCAT,
CACGAT, GACGCT, CGTCCG, CGAAGG, GTTGCG, GCCGTT,
ACGCGC, ACCGAC, TGTGCG, TCGTTA, TTTCGA, TAATCG,
GCGCCT, GCCGAT, TCGGTT, TACGAT, GTCCGC, AGCTCG,
TCGATG, TCACCG, TTCGGA, CAAGCG, CACGTT, AACGGC,
ATAGCG, GGTCGC, TCTCGC, AGTTCG, CGACCT, TGCCGG,
TTGGCG, GACTCG, AGCCCG, CCGCTT, AACACG, ACGAGA,
CCACGA, AGCGGA, CGCTCC, CTTCGA, AGGGCG, ATCCGT,
TGCGCC, TCGCAA, TTCTCG, AGACGC, GCGATT, AGGCGA,
AGCGAA, CATCGT, GACCGA, CGTTCC, TTCCCG, CGGGCC,
GCGGAA, CTCTCG, CGATTA, CGTCAC, CGCAGT, CATTCG,
TACGTT, CGAGAA, CGTACA, CCATCG, ACCGCG, GCCGCT,
GATCGG, GAAACG, ACGTGC, CTCGGA, TAAGCG, TCGACC,
TATCGT, CGCGGG, AGTCGT, GGACCG, CGCACA, CTGGCG,
CGGATA, CGTAGC, TCGGCC, GCGTCG, ACCGGC, CGGCAG,
TACGCC, ACCACG, ACGCTA, TCGCTG, CGCGCA, GTATCG,
CGTGAA, GACGCG, GCCCGA, AACGTA, AGTCGG, GCGGGA,
AAGCGT, CCGAGT, CGAAAG, CGAGTG, ACTACG, GCGCCG,
AATCGA, TTCGAA, TTGCGA, CCGACA, GCGCAC, TCGTTC,
TAACGA, CGACTT, ACGCTC, CGCGGT, ACGTAT, GCAACG,
ATAACG, TTACGG, AACGTC, TCCGTG, CAACGA, CGACAT,
CTGCGA, TGTCGA, TCCGGG, ATCCGG, CGCGAG, CGGCGG,
CGATTC, GCGAAA, CTCGAA, GTACGA, GAGCGC, CGGTAC,
CCGAAG, CTACGG, GACGAC, CCGGTG, AGTCGC, CGTCTT,
TCGTGG, CGTAAC, ACGGAA, AACCGA, CGCGTC, CCGGGT,
TCGTAC, AAGCCG, GGCGAA, GGGCGA, ACGATT, GGACGC,
CGCAAC, TCCGCA, TGACGG, CGGTGT, AGACCG, GCGTGC,
CCGGAG, GGTCGT, TCCGGT, CGGTCA, AATCGG, GCCGCG,
ACCGCT, CGCGTA, TATCGC, ACATCG, TACCGG, CGGCGT,
TGCCGT, GTAGCG, GACGGC, ATCCGC, TCTCCG, CGTTAA,
GGCTCG, ACCGAT, ACGCCT, CGATGG, CACCGG, CGACCC,
CGGATC, GCGCGC, GCCGAC, CGGCCA, ATTGCG, ACCGTT,
CGATAC, CATCGC, AACGCT, CGCTAA, ATGACG, CGTCCT,
ACAGCG, CGAAGT, GTCCGT, AGCGTG, TCGCGG, CGCAGC,
TCCGAG, GGCGGA, GCGAGA, GACACG, CCTCGA, CGAACA,
AAGTCG, CCGTCC, TTACGT, CGAGGG, GGTTCG, AACGCG,
TCCGTA, CTTCGG, CCGGTA, TCGCGT, CTCGTG, CGGCTC,
CGATGT, CACCGT, GACGTC, CGGTAT, TTCGTG, TACCGT,
ACAACG, GTAACG, CGTTTG, GCGTAT, CGATCA, GCGCTC,
TTTCGG, CCGTAA, CTACGT, TCGTGT, ACGCAC, TGGACG,
CGAGGT, CCGAGC, AACGAC, AAGCGC, TCGATC, TCGCCA,
ATACGA, CGAGCA, GTCCGG, CGGTTT, ACGAAA, GCGTTT,
CATCCG, TCGATA, CGCACG, GCGCTA, TTCGGG, GCCGGC,
CGCGGC, ACGTCG, GCCGTC, CGAGAG, TATCCG, CCGGCA,
CGTACG, CGTCAT, GATCGA, ACGCCG, TCGCAG, GCTACG,
CGGCTA, GAGCGT, ACGGGA, GGTCGG, GACGTA, ACCCGA,
GCGTCA, CGATTT, TTAACG, TCGAAC, AACGTG, CTTTCG,
CCGACG, TGCGAC, ACGGCC, TACGTC, CGATAT, CGAAAC,
TGGCGC, GGCCGC, GGACGT, GCGATC, TGCGCG, CGCACT,
CAACGG, ACCGGG, TACACG, GCGCCA, CGGTGC, GCGTGT,
AGTCGA, TCGGTC, CGCGCG, CGTGAG, ATCGCT, GGGACG,
CGGCGC, CGCGAC, TCGTAA, TCGGTA, AGCCGT, GACGGT,
AACGGG, GCCGTA, CCGGTC, ATGTCG, CTACGC, TAGCGT,
CGAGTA, ACTCCG, TCACGG, GACGCA, GCGCGT, CGTACT,
CCGAAC, CGAAGC, CGGAGA, GTCGCC, GCGCAG, CTTCGT,
CGTCCC, ATGCCG, ATCCGA, ACGCTG, CTCGAG, CGCTTG,
GATGCG, CCGGAC, CAACGT, CGCTGA, CGGTCG, GTCGTT,
GCGATA, GACGAG, CGTGTA, GCTAGC, TCTCGG, ACGGAT,
CGCGCT, TGAACG, GAGCGG, CGGCCG, CTCGGT, GCCGGT,
TCGTTG, TAGCGC, ACGATG, ACACCG, ACGGTT, TACGAC,
ACGTTA, AGTGCG, CGTTGA, CGCAAT, CGCTAG, CGCCGA,
CAGACG, GGACGG, CTCGCA, GCCGCA, TGCCGA, GTTACG,
CGATGC, CACCGC, CCGTTG, TTCCGT, TCGGGC, GCGTAC,
AAACCG, CGTTAG, CGTAAT, CGAACG, CTCGTA, TTAGCG,
ACGTTC, CTGCGT, TCGACG, TACGGC, ACCGTG, GTCGAT,
ATCGCG, CGAGTC, CGGAAA, GCGCGG, CGTGCA, CGGCAC,
TCACGT, ACTCGC, TCCCGC, TTATCG, TCCTCG, ACGATC,
AACGCA, ACGCGT, GCTCCG, CGCTTA, TCTTCG, GTGTCG,
CGATCG, ACCGTA, CACCCG, AACGGT, GACGGG, CGCGAT,
CACGGA, GGCCGT, TAAACG, GACGTG, TTACGA, CGTATG,
CGTGTC, CCTCGT, CGCACC, TATCGG, AATGCG, TCTCGT,
GCGCTG, GTCCGA, CGAGCG, GTGCCG, CGCGTT, CGCATG,
CTACCG, CGTTTA, CGAACT, ATCGCC, ACCGTC, TCGGAC,
CCTTCG, AGACGT, AGCCGC, CGCCAA, TGGTCG, CGAGAC,
CGTACC, CGGGAA, GCGGCC, CTCGTC, CCGACT, TCGGCG,
GAACCG, ACGTCA, CCCGGA, AGGACG, CATACG, TCGACT,
CTTCGC, GTCGCT, TCCGGA, GGTCGA, CGGATT, ACGCCA,
TGCGCT, CCGGCG, TACGCG, GTCGCG, CAGCGA, CACGAA,
TTTGCG, ACCGGT, TACGCT, CAACGC, CGGCAT, CCGCAA,
CGCGCC, CGTGAC, GCGTTC, TCGTGA, TTGACG, CGACGA,
ACGTAC, TGACGA, TATTCG, CGAAAT, GCTCGC, TTCCGC,
CGGCTT, TCGGCT, ACGCGG, ACCGAG, ACGCAG, TGCGAT,
GGTGCG, GCGTTA, TAGCCG, ATCGAT, GCACCG, GCGATG,
CCGTGA, CGTTTC, TACCGA, CTTCCG, AAGCGG, GCGGAT,
CTGCGC, CTCGAC, ACGATA, CCGGCT, AACGAG, TGAGCG,
TGCGTT, CGCTTC, ATCGTT, GCGACC, CGGTCT, CCGAAT,
CCGTAG, CCGCGA, CCCGAA, TAGTCG, ATTACG, CACTCG,
TCGCGA, TCCGAA, AGACGG, ACCGCA, GCGGTT, TGATCG,
TCACGC, TCGAAT, TCGTAG, GAACGC, CTCGCG, ACCCGA,
CGAGTT, CGCTAC, GACGAA, GAGCGA, CGAATG, ATGCGT,
ATCGTA, TTCGCG, CGAGAT, AGAACG, GCGCAA, CCGTTC,
TCGAGG, GGCGCC, GTCGGC, TCACGA, CCTCGC, ACTCGG,
CGCCGG, CGAACC, GCGGCT, CGGACA, GGACGA, TAACCG,
CGTTAC, CGTTGG, AGCGCT, GCGTGA, AATACG, GTTCCG,
CGTGCG, CCGTTA, CGATCT, TCAGCG, GTCGAC, TCCGTT,
GTGCGC, CGGAGT, CGACAA, ACGGAC, CCGGAT, GCGCGA,
GCCGAA, TTCCGA, CGGAAG, AACCGC, CGGGTG, GCGAAT,
AGGTCG, GCACGC, GCGTAG, TCGTCT, CCGACC, CGAGCT,
TGCGGG, TTGCCG, ACGTTG, ATCGCA, TCATCG, CCGGTT,
CCGATG, TCGCCT, GACTCG, TCCGAT, AAGACG, TTGTCG,
AAACGG, GTACCG, ATCGGT, GGCGTT, ATACGC, CGTATC,
ACGAAC, TCTGCG, ACGGTC, GGCGAT, GACGGA, CACGGG,
CTGTCG, CGAGCC, AGCGAC, AGGCGC, GACCCG, GGATCG,
CGGGGT, CGCCGT, TCGACA, CGTGCT, CTCCGA, TGCGCA,
CGCCAG, TCGGGG, GCTCGT, ATGCGG, ATCGAG, TCGAGT,
GGAGCG, TGCGGT, TTCGCT, TACGGG, ATTCGT, ACACGT,
GCTTCG, ACCCGC, CGTATA, GTCACG, TCGCAT, ACGGGC,
TCGCTT, CGCATA, TGTCCG, ACGACG, CGGTCC, GATACG,
TCGAAG, TCGGTG, GGCGCT, ATTTCG, GTTCGC, GCGACT,
GTCGTC, CTCGCT, CAACCG, TTTACG, TACGTG, GCGGCG,
TGGCGG, GCCGGA, AGCGCG, TGCGAG, CGTCGA, TCCGCC,
GGGTCG, ACGGCT, GACCGC, CGGTAA, GAACGT, TGCGTA,
CGGGTA, TGGCGT, CTCGTT, CGCCTA, TAGCGG, TACGAG,
GCGGAC, ATGCGC, ATCGAC, CTCGAT, TTCGTT, CACGAG,
TCTCGA, CAGCGG, CCGATA, ATTCCG, ACGTGA, GGCCGA,
GAGACG, GTACGC, TATGCG, GTCGGT, CCCGGT, CGTGAT,
AACTCG, CTTACG, TCGGAG, TTCGAT, GCGTTG, GTCGCA,
CGACGG, CCCGCA, GCTCGG, TCGCCC, ACGACC, CGTGTT,
CGATCC, ACGCAA, AGCGCC, CCGTAC, CGCTCA, GGAACG,
CGGAGC, AAGCGA, AACGAA, GTCGTA, GTGCGT, TCGTCC,
CGTCAA, GCACGT, AAACGC, CCGCGG, CGTTGT, CGGGCA,
CGCATC, CGACTG, CGTTCA, AGACGA, CGCTGT, GTTTCG,
TGCGGC, ATCGGC, GCGACG, ACCTCG, CGTCTG, CCGTCA,
TGCACG, GCGGGC, CGTTGC, CGACGT, CGCCGC, ATCACG,
ACTTCG, CGACAG, TACGTA, GAACGG, CCGATC, TCGAGC,
CGGACG, GGCGCG, ACCGGA, ACGGCG, TATCGA, ATTCGC,
CGCAGA, TTCGCC, ACGACT, ACGAAT, ACGTAG, CACGGT,
ATCGTC, ACACGC, AACCCG, TACGCA, ACGCGA, CGCTAT,
CGGAAC, ACCGAA, AAGGCG, AGATCG, GGGCGC, GGCGAC,
CACGCA, CGAATA, GCGAAC, AACGGA, TACGGT, CGTAGA,
AGCGAT, CCCGTA, CGGGTC, GCGGTC, CCGCGT, CTCGCC,
AGCGTT, TCGGCA, TGTACG, ATACCG, TTCCGG, AGAGCG,
GTGCGG, GTCGAG, CGCTTT, ACTCGT, GTTCGT, CGTTAT,
CATGCG, TCGGGT, TGCGTC, TCCCGT, GTCGTG, CACGTC,
GACCGT, CGACTA, GTTCGG, CCGTAT, GCGGTA, TCCACG,
CGGGAC, CTAACG, AAACGA, CGCCAC, AGCGGT, TTTTCG,
TCGCTA, GCGTAA, TGTCGG, ACTGCG, CCGCTC, CGGTTG,
TTCGAG, CGCAAA, TTGCGG, TTTCGT, GTACGT, GCGAGC,
ATACGG, CCGTTT, ACGGTG, ACGAAG, GCACGG, TCCGGC,
ATCGAA, GATCCG, CTCCGG, TGCCGC, ATGCGA, GGCACG,
CCGCTA, TCGTCA, GGCGGC, ACGCCC, CGTAAA, CATCGA,
CGAATC, AACGCC, CGACCA, TCTACG, GCCCGT, GCGGCA,
GGTACG, ACGACA, TTCGCA, CGATAA, CACGTA, ACGGGG,
TCCGTC, TTACGC, CGTCGG, ACCCGG, CAGCGT, ACGAGT,
TAACGG, CCTACG, TGACGT, TTCGGT, GTCGGG, AGCCCA,
CGCATT, TCCGAC, CGATTG, TGCTCG, AATCGT, ATCTCG,
TCGCGC, CGGAAT, CGGTAG, CGGCGA, CGCGAA, TAACGT,
TGTTCG, GCGGGT, GGCGTC, TACCGC, CGACGC, GCGGAG,
CCGTGC, ATCCCG, ACGTCT, ATGGCG, ACGAGG, TCGTGC,
CGTCGT, AGCGGG, AATTCG, CGAAGA, CCCGCG, ATCGGA,
TGTCGT, CGTATT, TATACG, CGTCCA, ACCGCC, TCGCTC,
CTAGCG, AGCGAG, CGCTCG, GGCGTA, TTGCGT, CACGGC,
TTCGTA, TCGTAT, ACGCAT, CGACTC, GGGCGT, CCGCGC,
TCGTTT, GACCGG, CCCGAC, GATCGC, AAATCG, AGTCCG,
AACGAT, TCGAGA, CGGGCG, CACACG, ATTCGA, CGGACT,
CGCGGA, ACGCTT, CGTTCG, TAGACG, TGCGGA, ACACGA,
GCGTCC, CGCCCG, AAAGCG, GCTCGA, CCGAGA, CGTCAG,
AACGTT, ACGAGC, TACGGA, GACGCC, CCGTCG, CGACAC,
TAGGCG, TCAACG, GCGCCC, TCGCAC, CGGACC, TTACCG,
AGCGGC, CGGCAA, CGTAGG, AGCACG, CTATCG, CCCCGA,
CGAAAA, ATCGGG, GGCGCA, TCCCGA, CACGCG, CGTTCT,
GCGAGT, TCGCCG, CGCTCT, TCGGGA, CGCAGG, TTTCGC,
CCGCCG, TACCCG, TTCGTC, AGTACG, GCGACA, ACGGCA,
TTCACG, TGACGC, GCTGCG, ACGTAA, CCGCAC, GGCGGT,
CCAACG, TCCGCG, GAACGA, ACGGTA, CGGGCT, CGTCTA,
ATTCGG, CCGAAA, GGCGAG, AACCGT, ATCGTG, GTCGAA,
AATCCG, GTGCGA, ACACGG, CGGTGA, TTCGGC, GCGGTG,
GCGAAG, TCGAAA, CTACGA, TGGCGA, TGCGAA, GTACGG,
CACGAC, CAGCGC, CTGACG, ATACGT, ACGGAG, CACGCT,
CGGTTC, GACGAT, GGTCCG, CGAATT, AATCGC, CTTGCG,
CCCGTT, GAATCG, AACCGG, TAACGC, CCCGAT, AGGCGT,
TACGAA, TAGCGA, GCGCAT, TCGATT, CGTAGT, AGCGTA,
GACGTT, CGTCGC, GAAGCG, ACTCGA, ACGTCC, TGTCGC,
GCACGA, GCGCTT, TCGGAA, CGCAAG, CAGTCG, GTTCGA,
CGCGTG, ACCCGT, CGGGAT, CGATGA, TCGTCG, TTCGAC,
CCGATT, ACGGGT, AGCGTC, TTGCGC, CCGGAA, CGTAAG,
GTCTCG, TACTCG, CGCCAT, CACCGA, TTTCCG, GATCGT,
GCATCG, CGAGGA, CGATAG, TGACCG, CCCGCT, CGCCTT,
CGGTTA, TCCGCT, GATTCG, GTCGGA, GCGAGG, CATCGG,
GTGGCG, GTCCCG, CAAACG, GCGTCT, CGGATG, CGGGTT,
and CGACCG.

9. A library of siRNA sequences, said library comprising a collection of siRNA sequences of at least 100 siRNAs that target at least 25 different genes, wherein said siRNA sequences comprise 18-30 bases, and at least 25% of the siRNA sequences have a hexamer sequence at positions 2-7 of an antisense sequence selected from the group consisting of the reverse complement of
GCAGCG, ATATCG, CAATCG, TCGGAT, GTGACG, CCGCAT,
CACGAT, GACGCT, CGTCCG, CGAAGG, GTTGCG, GCCGTT,
ACGCGC, ACCGAC, TGTGCG, TCGTTA, TTTCGA, TAATCG,
GCGCCT, GCCGAT, TCGGTT, TACGAT, GTCCGC, AGCTCG,
TCGATG, TCACCG, TTCGGA, CAAGCG, CACGTT, AACGGC,
ATAGCG, GGTCGC, TCTCGC, AGTTCG, CGACCT, TGCCGG,
TTGGCG, GAGTCG, AGCCCG, CCGCTT, AACACG, ACGAGA,
CCACGA, AGCGGA, CGCTCC, CTTCGA, AGGGCG, ATCCGT,
TGCGCC, TCGCAA, TTCTCG, AGACGC, GCGATT, AGGCGA,
AGCGAA, CATCGT, GACCGA, CGTTCC, TTCCCG, CGGGCC,
GCGGAA, CTCTCG, CGATTA, CGTCAC, CGCAGT, CATTCG,
TACGTT, CGAGAA, CGTACA, CCATCG, ACCGCG, GCCGCT,
GATCGG, GAAACG, ACGTGC, CTCGGA, TAAGCG, TCGACC,
TATCGT, CGCGGG, AGTCGT, GGACCG, CGCACA, CTGGCG,
CGGATA, CGTAGC, TCGGCC, GCGTCG, ACCGGC, CGGCAG,
TACGCC, ACCACG, ACGCTA, TCGCTG, CGCGCA, GTATCG,
CGTGAA, GACGCG, GCCCGA, AACGTA, AGTCGG, GCGGGA,
AAGCGT, CCGAGT, CGAAAG, CGAGTG, ACTACG, GCGCCG,
AATCGA, TTCGAA, TTGCGA, CCGACA, GCGCAC, TCGTTC,
TAACGA, CGACTT, ACGCTC, CGCGGT, ACGTAT, GCAACG,
ATAACG, TTACGG, AACGTC, TCCGTG, CAACGA, CGACAT,
CTGCGA, TGTCGA, TCCGGG, ATCCGG, CGCGAG, CGGCGG,
CGATTC, GCGAAA, CTCGAA, GTACGA, GAGCGC, CGGTAC,
CCGAAG, CTACGG, GACGAC, CCGGTG, AGTCGC, CGTCTT,
TCGTGG, CGTAAC, ACGGAA, AACCGA, CGCGTC, CCGGGT,
TCGTAC, AAGCCG, GGCGAA, GGGCGA, ACGATT, GGACGC,
CGCAAC, TCCGCA, TGACGG, CGGTGT, AGACCG, GCGTGC,
CCGGAG, GGTCGT, TCCGGT, CGGTCA, AATCGG, GCCGCG,
ACCGCT, CGCGTA, TATCGC, ACATCG, TACCGG, CGGCGT,
TGCCGT, GTAGCG, GACGGC, ATCCGC, TCTCCG, CGTTAA,
GGCTCG, ACCGAT, ACGCCT, CGATGG, CACCGG, CGACCC,
CGGATC, GCGCGC, GCCGAC, CGGCCA, ATTGCG, ACCGTT,
CGATAC, CATCGC, AACGCT, CGCTAA, ATGACG, CGTCCT,
ACAGCG, CGAAGT, GTCCGT, AGCGTG, TCGCGG, CGCAGC,
TCCGAG, GGCGGA, GCGAGA, GACACG, CCTCGA, CGAACA,
AAGTCG, CCGTCC, TTACGT, CGAGGG, GGTTCG, AACGCG,
TCCGTA, CTTCGG, CCGGTA, TCGCGT, CTCGTG, CGGCTC,
CGATGT, CACCGT, GACGTC, CGGTAT, TTCGTG, TACCGT,
ACAACG, GTAACG, CGTTTG, GCGTAT, CGATCA, GCGCTC,
TTTCGG, CCGTAA, CTACGT, TCGTGT, ACGCAC, TGGACG,
CGAGGT, CCGAGC, AACGAC, AAGCGC, TCGATC, TCGCCA,
ATACGA, CGAGCA, GTCCGG, CGGTTT, ACGAAA, GCGTTT,
CATCCG, TCGATA, CGCACG, GCGCTA, TTCGGG, GCCGGC,
CGCGGC, ACGTCG, GCCGTC, CGAGAG, TATCCG, CCGGCA,
CGTACG, CGTCAT, GATCGA, ACGCCG, TCGCAG, GCTACG,
CGGCTA, GAGCGT, ACGGGA, GGTCGG, GACGTA, ACCCGA,
GCGTCA, CGATTT, TTAACG, TCGAAC, AACGTG, CTTTCG,
CCGACG, TGCGAC, ACGGCC, TACGTC, CGATAT, CGAAAC,
TGGCGC, GGCCGC, GGACGT, GCGATC, TGCGCG, CGCACT,
CAACGG, ACCGGG, TACACG, GCGCCA, CGGTGC, GCGTGT,
AGTCGA, TCGGTC, CGCGCG, CGTGAG, ATCGCT, GGGACG,
CGGCGC, CGCGAC, TCGTAA, TCGGTA, AGCCGT, GACGGT,
AACGGG, GCCGTA, CCGGTC, ATGTCG, CTACGC, TAGCGT,
CGAGTA, ACTCCG, TCACGG, GACGCA, GCGCGT, CGTACT,
CCGAAC, CGAAGC, CGGAGA, GTCGCC, GCGCAG, CTTCGT,
CGTCCC, ATGCCG, ATCCGA, ACGCTG, CTCGAG, CGCTTG,
GATGCG, CCGGAC, CAACGT, CGCTGA, CGGTCG, GTCGTT,
GCGATA, GACGAG, CGTGTA, GCTAGC, TCTCGG, ACGGAT,
CGCGCT, TGAACG, GAGCGG, CGGCCG, CTCGGT, GCCGGT,
TCGTTG, TAGCGC, ACGATG, ACACCG, ACGGTT, TACGAC,
ACGTTA, AGTGCG, CGTTGA, CGCAAT, CGCTAG, CGCCGA,
CAGACG, GGACGG, CTCGCA, GCCGCA, TGCCGA, GTTACG,
CGATGC, CACCGC, CCGTTG, TTCCGT, TCGGGC, GCGTAC,
AAACCG, CGTTAG, CGTAAT, CGAACG, CTCGTA, TTAGCG,
ACGTTC, CTGCGT, TCGACG, TACGGC, ACCGTG, GTCGAT,
ATCGCG, CGAGTC, CGGAAA, GCGCGG, CGTGCA, CGGCAC,
TCACGT, ACTCGC, TCCCGC, TTATCG, TCCTCG, ACGATC,
AACGCA, ACGCGT, GCTCCG, CGCTTA, TCTTCG, GTGTCG,
CGATCG, ACCGTA, CACCCG, AACGGT, GACGGG, CGCGAT,
CACGGA, GGCCGT, TAAACG, GACGTG, TTACGA, CGTATG,
CGTGTC, CCTCGT, CGCACC, TATCGG, AATGCG, TCTCGT,
GCGCTG, GTCCGA, CGAGCG, GTGCCG, CGCGTT, CGCATG,
CTACCG, CGTTTA, CGAACT, ATCGCC, ACCGTC, TCGGAC,
CCTTCG, AGACGT, AGCCGC, CGCCAA, TGGTCG, CGAGAC,
CGTACC, CGGGAA, GCGGCC, CTCGTC, CCGACT, TCGGCG,
GAACCG, ACGTCA, CCCGGA, AGGACG, CATACG, TCGACT,
CTTCGC, GTCGCT, TCCGGA, GGTCGA, CGGATT, ACGCCA,
TGCGCT, CCGGCG, TACGCG, GTCGCG, CAGCGA, CACGAA,
TTTGCG, ACCGGT, TACGCT, CAACGC, CGGCAT, CCGCAA,
CGCGCC, CGTGAC, GCGTTC, TCGTGA, TTGACG, CGACGA,
ACGTAC, TGACGA, TATTCG, CGAAAT, GCTCGC, TTCCGC,
CGGCTT, TCGGCT, ACGCGG, ACCGAG, ACGCAG, TGCGAT,
GGTGCG, GCGTTA, TAGCCG, ATCGAT, GCACCG, GCGATG,
CCGTGA, CGTTTC, TACCGA, CTTCCG, AAGCGG, GCGGAT,
CTGCGC, CTCGAC, ACGATA, CCGGCT, AACGAG, TGAGCG,
TGCGTT, CGCTTC, ATCGTT, GCGACC, CGGTCT, CCGAAT,
CCGTAG, CCGCGA, CCCGAA, TAGTCG, ATTACG, CACTCG,
TCGCGA, TCCGAA, AGACGG, ACCGCA, GCGGTT, TGATCG,
TCACGC, TCGAAT, TCGTAG, GAACGC, CTCGCG, AGCCGA,
CGAGTT, CGCTAC, GACGAA, GAGCGA, CGAATG, ATGCGT,
ATCGTA, TTCGCG, CGAGAT, AGAACG, GCGCAA, CCGTTC,
TCGAGG, GGCGCC, GTCGGC, TCACGA, CCTCGC, ACTCGG,
CGCCGG, CGAACC, GCGGCT, CGGACA, GGACGA, TAACCG,
CGTTAC, CGTTGG, AGCGCT, GCGTGA, AATACG, GTTCCG,
CGTGCG, CCGTTA, CGATCT, TCAGCG, GTCGAC, TCCGTT,
GTGCGC, CGGAGT, CGACAA, ACGGAC, CCGGAT, GCGCGA,
GCCGAA, TTCCGA, CGGAAG, AACCGC, CGGGTG, GCGAAT,
AGGTCG, GCACGC, GCGTAG, TCGTCT, CCGACC, CGAGCT,
TGCGGG, TTGCCG, ACGTTG, ATCGCA, TCATCG, CCGGTT,
CCGATG, TCGCCT, GACTCG, TCCGAT, AAGACG, TTGTCG,
AAACGG, GTACCG, ATCGGT, GGCGTT, ATACGC, CGTATC,
ACGAAC, TCTGCG, ACGGTC, GGCGAT, GACGGA, CACGGG,
CTGTCG, CGAGCC, AGCGAC, AGGCGC, GACCCG, GGATCG,
CGGGGT, CGCCGT, TCGACA, CGTGCT, CTCCGA, TGCGCA,
CGCCAG, TCGGGG, GCTCGT, ATGCGG, ATCGAG, TCGAGT,
GGAGCG, TGCGGT, TTCGCT, TACGGG, ATTCGT, ACACGT,
GCTTCG, ACCCGC, CGTATA, GTCACG, TCGCAT, ACGGGC,
TCGCTT, CGCATA, TGTCCG, ACGACG, CGGTCC, GATACG,
TCGAAG, TCGGTG, GGCGCT, ATTTCG, GTTCGC, GCGACT,
GTCGTC, CTCGCT, CAACCG, TTTACG, TACGTG, GCGGCG,
TGGCGG, GCCGGA, AGCGCG, TGCGAG, CGTCGA, TCCGCC,
GGGTCG, ACGGCT, GACCGC, CGGTAA, GAACGT, TGCGTA,
CGGGTA, TGGCGT, CTCGTT, CGCCTA, TAGCGG, TACGAG,
GCGGAC, ATGCGC, ATCGAC, CTCGAT, TTCGTT, CACGAG,
TCTCGA, CAGCGG, CCGATA, ATTCCG, ACGTGA, GGCCGA,
GAGACG, GTACGC, TATGCG, GTCGGT, CCCGGT, CGTGAT,
AACTCG, CTTACG, TCGGAG, TTCGAT, GCGTTG, GTCGCA,
CGACGG, CCCGCA, GCTCGG, TCGCCC, ACGACC, CGTGTT,
CGATCC, ACGCAA, AGCGCC, CCGTAC, CGCTCA, GGAACG,
CGGAGC, AAGCGA, AACGAA, GTCGTA, GTGCGT, TCGTCC,
CGTCAA, GCACGT, AAACGC, CCGCGG, CGTTGT, CGGGCA,
CGCATC, CGACTG, CGTTCA, AGACGA, CGCTGT, GTTTCG,
TGCGGC, ATCGGC, GCGACG, ACCTCG, CGTCTG, CCGTCA,
TGCACG, GCGGGC, CGTTGC, CGACGT, CGCCGC, ATCACG,
ACTTCG, CGACAG, TACGTA, GAACGG, CCGATC, TCGAGC,
CGGACG, GGCGCG, ACCGGA, ACGGCG, TATCGA, ATTCGC,
CGCAGA, TTCGCC, ACGACT, ACGAAT, ACGTAG, CACGGT,
ATCGTC, ACACGC, AACCCG, TACGCA, ACGCGA, CGCTAT,
CGGAAC, ACCGAA, AAGGCG, AGATCG, GGGCGC, GGCGAC,
CACGCA, CGAATA, GCGAAC, AACGGA, TACGGT, CGTAGA,
AGCGAT, CCCGTA, CGGGTC, GCGGTC, CCGCGT, CTCGCC,
AGCGTT, TCGGCA, TGTACG, ATACCG, TTCCGG, AGAGCG,
GTGCGG, GTCGAG, CGCTTT, ACTCGT, GTTCGT, CGTTAT,
CATGCG, TCGGGT, TGCGTC, TCCCGT, GTCGTG, CACGTC,
GACCGT, CGACTA, GTTCGG, CCGTAT, GCGGTA, TCCACG,
CGGGAC, CTAACG, AAACGA, CGCCAC, AGCGGT, TTTTCG,
TCGCTA, GCGTAA, TGTCGG, ACTGCG, CCGCTC, CGGTTG,
TTCGAG, CGCAAA, TTGCGG, TTTCGT, GTACGT, GCGAGC,
ATACGG, CCGTTT, ACGGTG, ACGAAG, GCACGG, TCCGGC,
ATCGAA, GATCCG, CTCCGG, TGCCGC, ATGCGA, GGCACG,
CCGCTA, TCGTCA, GGCGGC, ACGCCC, CGTAAA, CATCGA,
CGAATC, AACGCC, CGACCA, TCTACG, GCCCGT, GCGGCA,
GGTACG, ACGACA, TTCGCA, CGATAA, CACGTA, ACGGGG,
TCCGTC, TTACGC, CGTCGG, ACCCGG, CAGCGT, ACGAGT,
TAACGG, CCTACG, TGACGT, TTCGGT, GTCGGG, AGCGCA,
CGCATT, TCCGAC, CGATTG, TGCTCG, AATCGT, ATCTCG,
TCGCGC, CGGAAT, CGGTAG, CGGCGA, CGCGAA, TAACGT,
TGTTCG, GCGGGT, GGCGTC, TACCGC, CGACGC, GCGGAG,
CCGTGC, ATCCCG, ACGTCT, ATGGCG, ACGAGG, TCGTGC,
CGTCGT, AGCGGG, AATTCG, CGAAGA, CCCGCG, ATCGGA,
TGTCGT, CGTATT, TATACG, CGTCCA, ACCGCC, TCGCTC,
CTAGCG, AGCGAG, CGCTCG, GGCGTA, TTGCGT, CACGGC,
TTCGTA, TCGTAT, ACGCAT, CGACTC, GGGCGT, CCGCGC,
TCGTTT, GACCGG, CCCGAC, GATCGC, AAATCG, AGTCCG,
AACGAT, TCGAGA, CGGGCG, CACACG, ATTCGA, CGGACT,
CGCGGA, ACGCTT, CGTTCG, TAGACG, TGCGGA, ACACGA,
GCGTCC, CGCCCG, AAAGCG, GCTCGA, CCGAGA, CGTCAG,
AACGTT, ACGAGC, TACGGA, GACGCC, CCGTCG, CGACAC,
TAGGCG, TCAACG, GCGCCC, TCGCAC, CGGACC, TTACCG,
AGCGGC, CGGCAA, CGTAGG, AGCACG, CTATCG, CCCCGA,
CGAAAA, ATCGGG, GGCGCA, TCCCGA, CACGCG, CGTTCT,
GCGACT, TCGCCG, CGCTCT, TCGGGA, CGCAGG, TTTCGC,
CCGCCG, TACCCG, TTCGTC, AGTACG, GCGACA, ACGGCA,
TTCACG, TGACGC, GCTGCG, ACGTAA, CCGCAC, GGCGGT,
CCAACG, TCCGCG, GAACGA, ACGGTA, CGGGCT, CGTCTA,
ATTCGG, CCGAAA, GGCGAG, AACCGT, ATCGTG, GTCGAA,
AATCCG, GTGCGA, ACACGG, CGGTGA, TTCGGC, GCGGTG,
GCGAAG, TCGAAA, CTACGA, TGGCGA, TGCGAA, GTACGG,
CACGAC, CAGCGC, CTGACG, ATACGT, ACGGAG, CACGCT,
CGGTTC, GACGAT, GGTCCG, CGAATT, AATCGC, CTTGCG,
CCCGTT, GAATCG, AACCGG, TAACGC, CCCGAT, AGGCGT,
TACGAA, TAGCGA, GCGCAT, TCGATT, CGTAGT, AGCGTA,
GACGTT, CGTCGC, GAAGCG, ACTCGA, ACGTCC, TGTCGC,
GCACGA, GCGCTT, TCGGAA, CGCAAG, CAGTCG, GTTCGA,
CGCGTG, ACCCGT, CGGGAT, CGATGA, TCGTCG, TTCGAC,
CCGATT, ACGGGT, AGCGTC, TTGCGC, CCGGAA, CGTAAG,
GTCTCG, TACTCG, CGCCAT, CACCGA, TTTCCG, GATCGT,
GCATCG, CGAGGA, CGATAG, TGACCG, CCCGCT, CGCCTT,
CGGTTA, TCCGCT, GATTCG, GTCGGA, GCGAGG, CATCGG,
GTGGCG, GTCCCG, CAAACG, GCGTCT, CGGATG, CGGGTT,
and CGACCG.

10. The library according to claim 9, wherein said set of siRNA sequences comprises sequences of at least 200 siRNAs.

11. The library according to claim 9, wherein said set of siRNA sequences targets at least 50 different genes.

12. The library according to claim 9, wherein at least 50% of the siRNA sequences have a hexamer sequence at positions 2-7 of said antisense sequence selected from group consisting of the reverse complement of
GCAGCG, ATATCG, CAATCG, TCGGAT, GTGACG, CCGCAT,
CACGAT, GACGCT, CGTCCG, CGAAGG, GTTGCG, GCCGTT,
ACGCGC, ACCGAC, TGTGCG, TCGTTA, TTTCGA, TAATCG,
GCGCCT, GCCGAT, TCGGTT, TACGAT, GTCCGC, AGCTCG,
TCGATG, TCACCG, TTCGGA, CAAGCG, CACGTT, AACGGC,
ATAGCG, GGTCGC, TCTCGC, AGTTCG, CGACCT, TGCCGG,
TTGGCG, GAGTCG, AGCCCG, CCGCTT, AACACG, ACGAGA,
CCACGA, AGCGGA, CGCTCC, CTTCGA, AGGGCG, ATCCGT,
TGCGCC, TCGCAA, TTCTCG, AGACGC, GCGATT, AGGCGA,
AGCGAA, CATCGT, GACCGA, CGTTCC, TTCCCG, CGGGCC,
GCGGAA, CTCTCG, CGATTA, CGTCAC, CGCAGT, CATTCG,
TACGTT, CGAGAA, CGTACA, CCATCG, ACCGCG, GCCGCT,
GATCGG, GAAACG, ACGTGC, CTCGGA, TAAGCG, TCGACC,
TATCGT, CGCGGG, AGTCGT, GGACCG, CGCACA, CTGGCG,
CGGATA, CGTAGC, TCGGCC, GCGTCG, ACCGGC, CGGCAG,
TACGCC, ACCACG, ACGCTA, TCGCTG, CGCGCA, GTATCG,
CGTGAA, GACGCG, GCCCGA, AACGTA, AGTCGG, GCGGGA,
AAGCGT, CCGAGT, CGAAAG, CGAGTG, ACTACG, GCGCCG,
AATCGA, TTCGAA, TTGCGA, CCGACA, GCGCAC, TCGTTC,
TAACGA, CGACTT, ACGCTC, CGCGGT, ACGTAT, GCAACG,
ATAACG, TTACGG, AACGTC, TCCGTG, CAACGA, CGACAT,
CTGCGA, TGTCGA, TCCGGG, ATCCGG, CGCGAG, CGGCGG,
CGATTC, GCGAAA, CTCGAA, GTACGA, GAGCGC, CGGTAC,
CCGAAG, CTACGG, GACGAC, CCGGTG, AGTCGC, CGTCTT,
TCGTGG, CGTAAC, ACGGAA, AACCGA, CGCGTC, CCGGGT,
TCGTAC, AAGCCG, GGCGAA, GGGCGA, ACGATT, GGACGC,
CGCAAC, TCCGCA, TGACGG, CGGTGT, AGACCG, GCGTGC,
CCGGAG, GGTCGT, TCCGGT, CGGTCA, AATCGG, GCCGCG,
ACCGCT, CGCGTA, TATCGC, ACATCG, TACCGG, CGGCGT,
TGCCGT, GTAGCG, GACGGC, ATCCGC, TCTCCG, CGTTAA,
GGCTCG, ACCGAT, ACGCCT, CGATGG, CACCGG, CGACCC,
CGGATC, GCGCGC, GCCGAC, CGGCCA, ATTGCG, ACCGTT,
CGATAC, CATCGC, AACGCT, CGCTAA, ATGACG, CGTCCT,
ACAGCG, CGAAGT, GTCCGT, AGCGTG, TCGCGG, CGCAGC,
TCCGAG, GGCGGA, GCGAGA, GACACG, CCTCGA, CGAACA,
AAGTCG, CCGTCC, TTACGT, CGAGGG, GGTTCG, AACGCG,
TCCGTA, CTTCGG, CCGGTA, TCGCGT, CTCGTG, CGGCTC,
CGATGT, CACCGT, GACGTC, CGGTAT, TTCGTG, TACCGT,
ACAACG, GTAACG, CGTTTG, GCGTAT, CGATCA, GCGCTC,
TTTCGG, CCGTAA, CTACGT, TCGTGT, ACGCAC, TGGACG,
CGAGGT, CCGAGC, AACGAC, AAGCGC, TCGATC, TCGCCA,
ATACGA, CGAGCA, GTCCGG, CGGTTT, ACGAAA, GCGTTT,
CATCCG, TCGATA, CGCACG, GCGCTA, TTCGGG, GCCGGC,
CGCGGC, ACGTCG, GCCGTC, CGAGAG, TATCCG, CCGGCA,
CGTACG, CGTCAT, GATCGA, ACGCCG, TCGCAG, GCTACG,
CGGCTA, GAGCGT, ACGGGA, GGTCGG, GACGTA, ACCCGA,
GCGTCA, CGATTT, TTAACG, TCGAAC, AACGTG, CTTTCG,
CCGACG, TGCGAC, ACGGCC, TACGTC, CGATAT, CGAAAC,
TGGCGC, GGCCGC, GGACGT, GCGATC, TGCGCG, CGCACT,
CAACGG, ACCGGG, TACACG, GCGCCA, CGGTGC, GCGTGT,
AGTCGA, TCGGTC, CGCGCG, CGTGAG, ATCGCT, GGGACG,
CGGCGC, CGCGAC, TCGTAA, TCGGTA, AGCCGT, GACGGT,
AACGGG, GCCGTA, CCGGTC, ATGTCG, CTACGC, TAGCGT,
CGAGTA, ACTCCG, TCACGG, GACGCA, GCGCGT, CGTACT,
CCGAAC, CGAAGC, CGGAGA, GTCGCC, GCGCAG, CTTCGT,
CGTCCC, ATGCCG, ATCCGA, ACGCTG, CTCGAG, CGCTTG,
GATGCG, CCGGAC, CAACGT, CGCTGA, CGGTCG, GTCGTT,
GCGATA, GACGAG, CGTGTA, GCTAGC, TCTCGG, ACGGAT,
CGCGCT, TGAACG, GAGCGG, CGGCCG, CTCGGT, GCCGGT,
TCGTTG, TAGCGC, ACGATG, ACACCG, ACGGTT, TACGAC,
ACGTTA, AGTGCG, CGTTGA, CGCAAT, CGCTAG, CGCCGA,
CAGACG, GGACGG, CTCGCA, GCCGCA, TGCCGA, GTTACG,
CGATGC, CACCGC, CCGTTG, TTCCGT, TCGGGC, GCGTAC,
AAACCG, CGTTAG, CGTAAT, CGAACG, CTCGTA, TTAGCG,
ACGTTC, CTGCGT, TCGACG, TACGGC, ACCGTG, GTCGAT,
ATCGCG, CGAGTC, CGGAAA, GCGCGG, CGTGCA, CGGCAC,
TCACGT, ACTCGC, TCCCGC, TTATCG, TCCTCG, ACGATC,
AACGCA, ACGCGT, GCTCCG, CGCTTA, TCTTCG, GTGTCG,
CGATCG, ACCGTA, CACCCG, AACGGT, GACGGG, CGCGAT,
CACGGA, GGCCGT, TAAACG, GACGTG, TTACGA, CGTATG,
CGTGTC, CCTCGT, CGCACC, TATCGG, AATGCG, TCTCGT,
GCGCTG, GTCCGA, CGAGCG, GTGCCG, CGCGTT, CGCATG,
CTACCG, CGTTTA, CGAACT, ATCGCC, ACCGTC, TCGGAC,
CCTTCG, AGACGT, AGCCGC, CGCCAA, TGGTCG, CGAGAC,
CGTACC, CGGGAA, GCGGCC, CTCGTC, CCGACT, TCGGCG,
GAACCG, ACGTCA, CCCGGA, AGGACG, CATACG, TCGACT,
CTTCGC, GTCGCT, TCCGGA, GGTCGA, CGGATT, ACGCCA,
TGCGCT, CCGGCG, TACGCG, GTCGCG, CAGCGA, CACGAA,
TTTGCG, ACCGGT, TACGCT, CAACGC, CGGCAT, CCGCAA,
CGCGCC, CGTGAC, GCGTTC, TCGTGA, TTGACG, CGACGA,
ACGTAC, TGACGA, TATTCG, CGAAAT, GCTCGC, TTCCGC,
CGGCTT, TCGGCT, ACGCGG, ACCGAG, ACGCAG, TGCGAT,
GGTGCG, GCGTTA, TAGCCG, ATCGAT, GCACCG, GCGATG,
CCGTGA, CGTTTC, TACCGA, CTTCCG, AAGCGG, GCGGAT,
CTGCGC, CTCGAC, ACGATA, CCGGCT, AACGAG, TGAGCG,
TGCGTT, CGCTTC, ATCGTT, GCGACC, CGGTCT, CCGAAT,
CCGTAG, CCGCGA, CCCGAA, TAGTCG, ATTACG, CACTCG,
TCGCGA, TCCGAA, AGACGG, ACCGCA, GCGGTT, TGATCG,
TCACGC, TCGAAT, TCGTAG, GAACGC, CTCGCG, AGCCGA,
CGAGTT, CGCTAC, GACGAA, GAGCGA, CGAATG, ATGCGT,
ATCGTA, TTCGCG, CGAGAT, AGAACG, GCGCAA, CCGTTC,
TCGAGG, GGCGCC, GTCGGC, TCACGA, CCTCGC, ACTCGG,
CGCCGG, CGAACC, GCGGCT, CGGACA, GGACGA, TAACCG,
CGTTAC, CGTTGG, AGCGCT, GCGTGA, AATACG, GTTCCG,
CGTGCG, CCGTTA, CGATCT, TCAGCG, GTCGAC, TCCGTT,
GTGCGC, CGGAGT, CGACAA, ACGGAC, CCGGAT, GCGCGA,
GCCGAA, TTCCGA, CGGAAG, AACCGC, CGGGTG, GCGAAT,
AGGTCG, GCACGC, GCGTAG, TCGTCT, CCGACC, CGAGCT,
TGCGGG, TTGCCG, ACGTTG, ATCGCA, TCATCG, CCGGTT,
CCGATG, TCGCCT, GACTCG, TCCGAT, AAGACG, TTGTCG,
AAACGG, GTACCG, ATCGGT, GGCGTT, ATACGC, CGTATC,
ACGAAC, TCTGCG, ACGGTC, GGCGAT, GACGGA, CACGGG,
CTGTCG, CGAGCC, AGCGAC, AGGCGC, GACCCG, GGATCG,
CGGGGT, CGCCGT, TCGACA, CGTGCT, CTCCGA, TGCGCA,
CGCCAG, TCGGGG, GCTCGT, ATGCGG, ATCGAG, TCGAGT,
GGAGCG, TGCGGT, TTCGCT, TACGGG, ATTCGT, ACACGT,
GCTTCG, ACCCGC, CGTATA, GTCACG, TCGCAT, ACGGGC,
TCGCTT, CGCATA, TGTCCG, ACGACG, CGGTCC, GATACG,
TCGAAG, TCGGTG, GGCGCT, ATTTCG, GTTCGC, GCGACT,
GTCGTC, CTCGCT, CAACCG, TTTACG, TACGTG, GCGGCG,
TGGCGG, GCCGGA, AGCGCG, TGCGAG, CGTCGA, TCCGCC,
GGGTCG, ACGGCT, GACCGC, CGGTAA, GAACGT, TGCGTA,
CGGGTA, TGGCGT, CTCGTT, CGCCTA, TAGCGG, TACGAG,
GCGGAC, ATGCGC, ATCGAC, CTCGAT, TTCGTT, CACGAG,
TCTCGA, CAGCGG, CCGATA, ATTCCG, ACGTGA, GGCCGA,
GAGACG, GTACGC, TATGCG, GTCGGT, CCCGGT, CGTGAT,
AACTCG, CTTACG, TCGGAG, TTCGAT, GCGTTG, GTCGCA,
CGACGG, CCCGCA, GCTCGG, TCGCCC, ACGACC, CGTGTT,
CGATCC, ACGCAA, AGCGCC, CCGTAC, CGCTCA, GGAACG,
CGGAGC, AAGCGA, AACGAA, GTCGTA, GTGCGT, TCGTCC,
CGTCAA, GCACGT, AAACGC, CCGCGG, CGTTGT, CGGGCA,
CGCATC, CGACTG, CGTTCA, AGACGA, CGCTGT, GTTTCG,
TGCGGC, ATCGGC, GCGACG, ACCTCG, CGTCTG, CCGTCA,
TGCACG, GCGGGC, CGTTGC, CGACGT, CGCCGC, ATCACG,
ACTTCG, CGACAG, TACGTA, GAACGG, CCGATC, TCGAGC,
CGGACG, GGCGCG, ACCGGA, ACGGCG, TATCGA, ATTCGC,
CGCAGA, TTCGCC, ACGACT, ACGAAT, ACGTAG, CACGGT,
ATCGTC, ACACGC, AACCCG, TACGCA, ACGCGA, CGCTAT,
CGGAAC, ACCGAA, AAGGCG, AGATCG, GGGCGC, GGCGAC,
CACGCA, CGAATA, GCGAAC, AACGGA, TACGGT, CGTAGA,
AGCGAT, CCCGTA, CGGGTC, GCGGTC, CCGCGT, CTCGCC,
AGCGTT, TCGGCA, TGTACG, ATACCG, TTCCGG, AGAGCG,
GTGCGG, GTCGAG, CGCTTT, ACTCGT, GTTCGT, CGTTAT,
CATGCG, TCGGGT, TGCGTC, TCCCGT, GTCGTG, CACGTC,
GACCGT, CGACTA, GTTCGG, CCGTAT, GCGGTA, TCCACG,
CGGGAC, CTAACG, AAACGA, CGCCAC, AGCGGT, TTTTCG,
TCGCTA, GCGTAA, TGTCGG, ACTGCG, CCGCTC, CGGTTG,
TTCGAG, CGCAAA, TTGCGG, TTTCGT, GTACGT, GCGAGC,
ATACGG, CCGTTT, ACGGTG, ACGAAG, GCACGG, TCCGGC,
ATCGAA, GATCCG, CTCCGG, TGCCGC, ATGCGA, GGCACG,
CCGCTA, TCGTCA, GGCGGC, ACGCCC, CGTAAA, CATCGA,
CGAATC, AACGCC, CGACCA, TCTACG, GCCCGT, GCGGCA,
GGTACG, ACGACA, TTCGCA, CGATAA, CACGTA, ACGGGG,
TCCGTC, TTACGC, CGTCGG, ACCCGG, CAGCGT, ACGAGT,
TAACGG, CCTACG, TGACGT, TTCGGT, GTCGGG, AGCGCA,
CGCATT, TCCGAC, CGATTG, TGCTCG, AATCGT, ATCTCG,
TCGCGC, CGGAAT, CGGTAG, CGGCGA, CGCGAA, TAACGT,
TGTTCG, GCGGGT, GGCGTC, TACCGC, CGACGC, GCGGAG,
CCGTGC, ATCCCG, ACGTCT, ATGGCG, ACGAGG, TCGTGC,
CGTCGT, AGCGGG, AATTCG, CGAAGA, CCCGCG, ATCGGA,
TGTCGT, CGTATT, TATACG, CGTCCA, ACCGCC, TCGCTC,
CTAGCG, AGCGAG, CGCTCG, GGCGTA, TTGCGT, CACGGC,
TTCGTA, TCGTAT, ACGCAT, CGACTC, GGGCGT, CCGCGC,
TCGTTT, GACCGG, CCCGAC, GATCGC, AAATCG, AGTCCG,
AACGAT, TCGAGA, CGGGCG, CACACG, ATTCGA, CGGACT,
CGCGGA, ACGCTT, CGTTCG, TAGACG, TGCGGA, ACACGA,
GCGTCC, CGCCCG, AAAGCG, GCTCGA, CCGAGA, CGTCAG,
AACGTT, ACGAGC, TACGGA, GACGCC, CCGTCG, CGACAC,
TAGGCG, TCAACG, GCGCCC, TCGCAC, CGGACC, TTACCG,
AGCGGC, CGGCAA, CGTAGG, AGCACG, CTATCG, CCCCGA,
CGAAAA, ATCGGG, GGCGCA, TCCCGA, CACGCG, CGTTCT,
GCGAGT, TCGCCG, CGCTCT, TCGGGA, CGCAGG, TTTCGC,
CCGCCG, TACCCG, TTCGTC, AGTACG, GCGACA, ACGGCA,
TTCACG, TGACGC, GCTGCG, ACGTAA, CCGCAC, GGCGGT,
CCAACG, TCCGCG, GAACGA, ACGGTA, CGGGCT, CGTCTA,
ATTCGG, CCGAAA, GGCGAG, AACCGT, ATCGTG, GTCGAA,
AATCCG, GTGCGA, ACACGG, CGGTGA, TTCGGC, GCGGTG,
GCGAAG, TCGAAA, CTACGA, TGGCGA, TGCGAA, GTACGG,
CACGAC, CAGCGC, CTGACG, ATACGT, ACGGAG, CACGCT,
CGGTTC, GACGAT, GGTCCG, CGAATT, AATCGC, CTTGCG,
CCCGTT, GAATCG, AACCGG, TAACGC, CCCGAT, AGGCGT,
TACGAA, TAGCGA, GCGCAT, TCGATT, CGTAGT, AGCGTA,
GACGTT, CGTCGC, GAAGCG, ACTCGA, ACGTCC, TGTCGC,
GCACGA, GCGCTT, TCGGAA, CGCAAG, CAGTCG, GTTCGA,
CGCGTG, ACCCGT, CGGGAT, CGATGA, TCGTCG, TTCGAC,
CCGATT, ACGGGT, AGCGTC, TTGCGC, CCGGAA, CGTAAG,
GTCTCG, TACTCG, CGCCAT, CACCGA, TTTCCG, GATCGT,
GCATCG, CGAGGA, CGATAG, TGACCG, CCCGCT, CGCCTT,
CGGTTA, TCCGCT, GATTCG, GTCGGA, GCGAGG, CATCGG,
GTGGCG, GTCCCG, CAAACG, GCGTCT, CGGATG, CGGGTT,
and CGACCG.

13. A computer program product for use in conjunction with a computer system, the computer program product comprising a computer readable storage medium and a computer program mechanism embedded therein, the computer program mechanism comprising:
a. an input module, wherein said input module permits a user to identify a target sequence;
b. a database mining module, wherein said database mining module is coupled to said input module and is capable of searching a siRNA database comprised of siRNA sequences targeting at least 25 different genes, wherein said siRNA sequences comprise 18-30 bases, and
c. an output module, wherein said output module is coupled to said siRNA database mining module and said output module is capable of providing to said user an identification of one or more siRNA sequences from said database where each siRNA that is identified comprises an antisense sequence that is at least 80% complementary to a region of said target sequence and at least 25% of the siRNA sequences identified from said database have a hexamer sequence at positions 2-7 of said antisense sequence selected from the group consisting of the reverse complement of
GCAGCG, ATATCG, CAATCG, TCGGAT, GTGACG, CCGCAT,
CACGAT, GACGCT, CGTCCG, CGAAGG, GTTGCG, GCCGTT,
ACGCGC, ACCGAC, TGTGCG, TCGTTA, TTTCGA, TAATCG,
GCGCCT, GCCGAT, TCGGTT, TACGAT, GTCCGC, AGCTCG,
TCGATG, TCACCG, TTCGGA, CAAGCG, CACGTT, AACGGC,
ATAGCG, GGTCGC, TCTCGC, AGTTCG, CGACCT, TGCCGG,
TTGGCG, GAGTCG, AGCCCG, CCGCTT, AACACG, ACGAGA,
CCACGA, AGCGGA, CGCTCC, CTTCGA, AGGGCG, ATCCGT,
TGCGCC, TCGCAA, TTCTCG, AGACGC, GCGATT, AGGCGA,
AGCGAA, CATCGT, GACCGA, CGTTCC, TTCCCG, CGGGCC,
GCGGAA, CTCTCG, CGATTA, CGTCAC, CGCAGT, CATTCG,
TACGTT, CGAGAA, CGTACA, CCATCG, ACCGCG, GCCGCT,
GATCGG, GAAACG, ACGTGC, CTCGGA, TAAGCG, TCGACC,
TATCGT, CGCGGG, AGTCGT, GGACCG, CGCACA, CTGGCG,
CGGATA, CGTAGC, TCGGCC, GCGTCG, ACCGGC, CGGCAG,
TACGCC, ACCACG, ACGCTA, TCGCTG, CGCGCA, GTATCG,
CGTGAA, GACGCG, GCCCGA, AACGTA, AGTCGG, GCGGGA,
AAGCGT, CCGAGT, CGAAAG, CGAGTG, ACTACG, GCGCCG,
AATCGA, TTCGAA, TTGCGA, CCGACA, GCGCAC, TCGTTC,
TAACGA, CGACTT, ACGCTC, CGCGGT, ACGTAT, GCAACG,
ATAACG, TTACGG, AACGTC, TCCGTG, CAACGA, CGACAT,
CTGCGA, TGTCGA, TCCGGG, ATCCGG, CGCGAG, CGGCGG,
CGATTC, GCGAAA, CTCGAA, GTACGA, GAGCGC, CGGTAC,
CCGAAG, CTACGG, GACGAC, CCGGTG, AGTCGC, CGTCTT,
TCGTGG, CGTAAC, ACGGAA, AACCGA, CGCGTC, CCGGGT,
TCGTAC, AAGCCG, GGCGAA, GGGCGA, ACGATT, GGACGC,
CGCAAC, TCCGCA, TGACGG, CGGTGT, AGACCG, GCGTGC,
CCGGAG, GGTCGT, TCCGGT, CGGTCA, AATCGG, GCCGCG,
ACCGCT, CGCGTA, TATCGC, ACATCG, TACCGG, CGGCGT,
TGCCGT, GTAGCG, GACGGC, ATCCGC, TCTCCG, CGTTAA,
GGCTCG, ACCGAT, ACGCCT, CGATGG, CACCGG, CGACCC,
CGGATC, GCGCGC, GCCGAC, CGGCCA, ATTGCG, ACCGTT,
CGATAC, CATCGC, AACGCT, CGCTAA, ATGACG, CGTCCT,
ACAGCG, CGAAGT, GTCCGT, AGCGTG, TCGCGG, CGCAGC,
TCCGAG, GGCGGA, GCGAGA, GACACG, CCTCGA, CGAACA,
AAGTCG, CCGTCC, TTACGT, CGAGGG, GGTTCG, AACGCG,
TCCGTA, CTTCGG, CCGGTA, TCGCGT, CTCGTG, CGGCTC,
CGATGT, CACCGT, GACGTC, CGGTAT, TTCGTG, TACCGT,
ACAACG, GTAACG, CGTTTG, GCGTAT, CGATCA, GCGCTC,
TTTCGG, CCGTAA, CTACGT, TCGTGT, ACGCAC, TGGACG,
CGAGGT, CCGAGC, AACGAC, AAGCGC, TCGATC, TCGCCA,
ATACGA, CGAGCA, GTCCGG, CGGTTT, ACGAAA, GCGTTT,
CATCCG, TCGATA, CGCACG, GCGCTA, TTCGGG, GCCGGC,
CGCGGC, ACGTCG, GCCGTC, CGAGAG, TATCCG, CCGGCA,
CGTACG, CGTCAT, GATCGA, ACGCCG, TCGCAG, GCTACG,
CGGCTA, GAGCGT, ACGGGA, GGTCGG, GACGTA, ACCCGA,
GCGTCA, CGATTT, TTAACG, TCGAAC, AACGTG, CTTTCG,
CCGACG, TGCGAC, ACGGCC, TACGTC, CGATAT, CGAAAC,
TGGCGC, GGCCGC, GGACGT, GCGATC, TGCGCG, CGCACT,
CAACGG, ACCGGG, TACACG, GCGCCA, CGGTGC, GCGTGT,
AGTCGA, TCGGTC, CGCGCG, CGTGAG, ATCGCT, GGGACG,
CGGCGC, CGCGAC, TCGTAA, TCGGTA, AGCCGT, GACGGT,
AACGGG, GCCGTA, CCGGTC, ATGTCG, CTACGC, TAGCGT,
CGAGTA, ACTCCG, TCACGG, GACGCA, GCGCGT, CGTACT,
CCGAAC, CGAAGC, CGGAGA, GTCGCC, GCGCAG, CTTCGT,
CGTCCC, ATGCCG, ATCCGA, ACGCTG, CTCGAG, CGCTTG,
GATGCG, CCGGAC, CAACGT, CGCTGA, CGGTCG, GTCGTT,
GCGATA, GACGAG, CGTGTA, GCTAGC, TCTCGG, ACGGAT,
CGCGCT, TGAACG, GAGCGG, CGGCCG, CTCGGT, GCCGGT,
TCGTTG, TAGCGC, ACGATG, ACACCG, ACGGTT, TACGAC,
ACGTTA, AGTGCG, CGTTGA, CGCAAT, CGCTAG, CGCCGA,
CAGACG, GGACGG, CTCGCA, GCCGCA, TGCCGA, GTTACG,
CGATGC, CACCGC, CCGTTG, TTCCGT, TCGGGC, GCGTAC,
AAACCG, CGTTAG, CGTAAT, CGAACG, CTCGTA, TTAGCG,
ACGTTC, CTGCGT, TCGACG, TACGGC, ACCGTG, GTCGAT,
ATCGCG, CGAGTC, CGGAAA, GCGCGG, CGTGCA, CGGCAC,
TCACGT, ACTCGC, TCCCGC, TTATCG, TCCTCG, ACGATC,
AACGCA, ACGCGT, GCTCCG, CGCTTA, TCTTCG, GTGTCG,
CGATCG, ACCGTA, CACCCG, AACGGT, GACGGG, CGCGAT,
CACGGA, GGCCGT, TAAACG, GACGTG, TTACGA, CGTATG,
CGTGTC, CCTCGT, CGCACC, TATCGG, AATGCG, TCTCGT,
GCGCTG, GTCCGA, CGACCG, GTGCCG, CGCGTT, CGCATG,
CTACCG, CGTTTA, CGAACT, ATCGCC, ACCGTC, TCGGAC,
CCTTCG, AGACGT, AGCCGC, CGCCAA, TGGTCG, CGAGAC,
CGTACC, CGGGAA, GCGGCC, CTCGTC, CCGACT, TCGGCG,
GAACCG, ACGTCA, CCCGGA, AGGACG, CATACG, TCGACT,
CTTCGC, GTCGCT, TCCGGA, GGTCGA, CGGATT, ACGCCA,
TGCGCT, CCGGCG, TACGCG, GTCGCG, CAGCGA, CACGAA,
TTTGCG, ACCGGT, TACGCT, CAACGC, CGGCAT, CCGCAA,
CGCGCC, CGTGAC, GCGTTC, TCGTGA, TTGACG, CGACGA,
ACGTAC, TGACGA, TATTCG, CGAAAT, GCTCGC, TTCCGC,
CGGCTT, TCGGCT, ACGCGG, ACCGAG, ACGCAG, TGCGAT,
GGTGCG, GCGTTA, TAGCCG, ATCGAT, GCACCG, GCGATG,
CCGTGA, CGTTTC, TACCGA, CTTCCG, AAGCGG, GCGGAT,
CTGCGC, CTCGAC, ACGATA, CCGGCT, AACGAG, TGAGCG,
TGCGTT, CGCTTC, ATCGTT, GCGACC, CGGTCT, CCGAAT,
CCGTAG, CCGCGA, CCCGAA, TAGTCG, ATTACG, CACTCG,
TCGCGA, TCCGAA, AGACGG, ACCGCA, GCGGTT, TGATCG,
TCACGC, TCGAAT, TCGTAG, GAACGC, CTCGCG, AGCCGA,
CGAGTT, CGCTAC, GACGAA, GAGCGA, CGAATG, ATGCGT,
ATCGTA, TTCGCG, CGAGAT, AGAACG, GCGCAA, CCGTTC,
TCGAGG, GGCGCC, GTCGGC, TCACGA, CCTCGC, ACTCGG,
CGCCGG, CGAACC, GCGGCT, CGGACA, GGACGA, TAACCG,
CGTTAC, CGTTGG, AGCGCT, GCGTGA, AATACG, GTTCCG,
CGTGCG, CCGTTA, CGATCT, TCAGCG, GTCGAC, TCCGTT,
GTGCGC, CGGAGT, CGACAA, ACGGAC, CCGGAT, GCGCGA,
GCCGAA, TTCCGA, CGGAAG, AACCGC, CGGGTG, GCGAAT,
AGGTCG, GCACGC, GCGTAG, TCGTCT, CCGACC, CGAGCT,
TGCGGG, TTGCCG, ACGTTG, ATCGCA, TCATCG, CCGGTT,
CCGATG, TCGCCT, GACTCG, TCCGAT, AAGACG, TTGTCG,
AAACGG, GTACCG, ATCGGT, GGCGTT, ATACGC, CGTATC,
ACGAAC, TCTGCG, ACGGTC, GGCGAT, GACGGA, CACGGG,
CTGTCG, CGAGCC, AGCGAC, AGGCGC, GACCCG, GGATCG,
CGGGGT, CGCCGT, TCGACA, CGTGCT, CTCCGA, TGCGCA,
CGCCAG, TCGGGG, GCTCGT, ATGCGG, ATCGAG, TCGAGT,
GGAGCG, TGCGGT, TTCGCT, TACGGG, ATTCGT, ACACGT,
GCTTCG, ACCCGC, CGTATA, GTCACG, TCGCAT, ACGGGC,
TCGCTT, CGCATA, TGTCCG, ACGACG, CGGTCC, GATACG,
TCGAAG, TCGGTG, GGCGCT, ATTTCG, GTTCGC, GCGACT,
GTCGTC, CTCGCT, CAACCG, TTTACG, TACGTG, GCGGCG,
TGGCGG, GCCGGA, AGCGCG, TGCGAG, CGTCGA, TCCGCC,
GGGTCG, ACGGCT, GACCGC, CGGTAA, GAACGT, TGCGTA,
CGGGTA, TGGCGT, CTCGTT, CGCCTA, TAGCGG, TACGAG,
GCGGAC, ATGCGC, ATCGAC, CTCGAT, TTCGTT, CACGAG,
TCTCGA, CAGCGG, CCGATA, ATTCCG, ACGTGA, GGCCGA,
GAGACG, GTACGC, TATGCG, GTCGGT, CCCGGT, CGTGAT,
AACTCG, CTTACG, TCGGAG, TTCGAT, GCGTTG, GTCGCA,
CGACGG, CCCGCA, GCTCGG, TCGCCC, ACGACC, CGTGTT,
CGATCC, ACGCAA, AGCGCC, CCGTAC, CGCTCA, GGAACG,
CGGAGC, AAGCGA, AACGAA, GTCGTA, GTGCGT, TCGTCC,
CGTCAA, GCACGT, AAACGC, CCGCGG, CGTTGT, CGGGCA,
CGCATC, CGACTG, CGTTCA, AGACGA, CGCTGT, GTTTCG,
TGCGGC, ATCGGC, GCGACG, ACCTCG, CGTCTG, CCGTCA,
TGCACG, GCGGGC, CGTTGC, CGACGT, CGCCGC, ATCACG,
ACTTCG, CGACAG, TACGTA, GAACGG, CCGATC, TCGAGC,
CGGACG, GGCGCG, ACCGGA, ACGGCG, TATCGA, ATTCGC,
CGCAGA, TTCGCC, ACGACT, ACGAAT, ACGTAG, CACGGT,
ATCGTC, ACACGC, AACCCG, TACGCA, ACGCGA, CGCTAT,
CGGAAC, ACCGAA, AAGGCG, AGATCG, GGGCGC, GGCGAC,
CACGCA, CGAATA, GCGAAC, AACGGA, TACGGT, CGTAGA,
AGCGAT, CCCGTA, CGGGTC, GCGGTC, CCGCGT, CTCGCC,
AGCGTT, TCGGCA, TGTACG, ATACCG, TTCCGG, AGAGCG,
GTGCGG, GTCGAG, CGCTTT, ACTCGT, GTTCGT, CGTTAT,
CATGCG, TCGGGT, TGCGTC, TCCCGT, GTCGTG, CACGTC,
GACCGT, CGACTA, GTTCGG, CCGTAT, GCGGTA, TCCACG,
CGGGAC, CTAACG, AAACGA, CGCCAC, AGCGGT, TTTTCG,
TCGCTA, GCGTAA, TGTCGG, ACTGCG, CCGCTC, CGGTTG,
TTCGAG, CGCAAA, TTGCGG, TTTCGT, GTACGT, GCGAGC,
ATACGG, CCGTTT, ACGGTG, ACGAAG, GCACGG, TCCGGC,
ATCGAA, GATCCG, CTCCGG, TGCCGC, ATGCGA, GGCACG,
CCGCTA, TCGTCA, GGCGGC, ACGCCC, CGTAAA, CATCGA,
CGAATC, AACGCC, CGACCA, TCTACG, GCCCGT, GCGGCA,
GGTACG, ACGACA, TTCGCA, CGATAA, CACGTA, ACGGGG,
TCCGTC, TTACGC, CGTCGG, ACCCGG, CAGCGT, ACGAGT,
TAACGG, CCTACG, TGACGT, TTCGGT, GTCGGG, AGCGCA,
CGCATT, TCCGAC, CGATTG, TGCTCG, AATCGT, ATCTCG,
TCGCGC, CGGAAT, CGGTAG, CGGCGA, CGCGAA, TAACGT,
TGTTCG, GCGGGT, GGCGTC, TACCGC, CGACGC, GCGGAG,
CCGTGC, ATCCCG, ACGTCT, ATGGCG, ACGAGG, TCGTGC,
CGTCGT, AGCGGG, AATTCG, CGAAGA, CCCGCG, ATCGGA,
TGTCGT, CGTATT, TATACG, CGTCCA, ACCGCC, TCGCTC,
CTAGCG, AGCGAG, CGCTCG, GGCGTA, TTGCGT, CACGGC,
TTCGTA, TCGTAT, ACGCAT, CGACTC, GGGCGT, CCGCGC,
TCGTTT, GACCGG, CCCGAC, GATCGC, AAATCG, AGTCCG,
AACGAT, TCGAGA, CGGGCG, CACACG, ATTCGA, CGGACT,
CGCGGA, ACGCTT, CGTTCG, TAGACG, TGCGGA, ACACGA,
GCGTCC, CGCCCG, AAAGCG, GCTCGA, CCGAGA, CGTCAG,
AACGTT, ACGAGC, TACGGA, GACGCC, CCGTCG, CGACAC,
TAGGCG, TCAACG, GCGCCC, TCGCAC, CGGACC, TTACCG,
AGCGGC, CGGCAA, CGTAGG, AGCACG, CTATCG, CCCCGA,
CGAAAA, ATCGGG, GGCGCA, TCCCGA, CACGCG, CGTTCT,
GCGAGT, TCGCCG, CGCTCT, TCGGGA, CGCAGG, TTTCGC,
CCGCCG, TACCCG, TTCGTC, AGTACG, GCGACA, ACGGCA,
TTCACG, TGACGC, GCTGCG, ACGTAA, CCGCAC, GGCGGT,
CCAACG, TCCGCG, GAACGA, ACGGTA, CGGGCT, CGTCTA,
ATTCGG, CCGAAA, GGCGAG, AACCGT, ATCGTG, GTCGAA,
AATCCG, GTGCGA, ACACGG, CGGTGA, TTCGGC, GCGGTG,
GCGAAG, TCGAAA, CTACGA, TGGCGA, TGCGAA, GTACGG,
CACGAC, CAGCGC, CTGACG, ATACGT, ACGGAG, CACGCT,
CGGTTC, GACGAT, GGTCCG, CGAATT, AATCGC, CTTGCG,
CCCGTT, GAATCG, AACCGG, TAACGC, CCCGAT, AGGCGT,
TACGAA, TAGCGA, GCGCAT, TCGATT, CGTAGT, AGCGTA,
GACGTT, CGTCGC, GAAGCG, ACTCGA, ACGTCC, TGTCGC,
GCACGA, GCGCTT, TCGGAA, CGCAAG, CAGTCG, GTTCGA,
CGCGTG, ACCCGT, CGGGAT, CGATGA, TCGTCG, TTCGAC,
CCGATT, ACGGGT, AGCGTC, TTGCGC, CCGGAA, CGTAAG,
GTCTCG, TACTCG, CGCCAT, CACCGA, TTTCCG, GATCGT,
GCATCG, CGAGGA, CGATAG, TGACCG, CCCGCT, CGCCTT,
CGGTTA, TCCGCT, GATTCG, GTCGGA, GCGAGG, CATCGG,
GTGGCG, GTCCCG, CAAACG, GCCTCT, CGGATG, CGGGTT,
and CGACCG.

14. The computer program product of claim 13 further comprising said siRNA database.


SEQUENCE LISTING (12 sequences)

SEQ ID NO: 1 - RNA, 19 nt, Artificial Sequence
  Synthetic
gaaagagcaucuacgguga

SEQ ID NO: 2 - RNA, 19 nt, Artificial Sequence
  Synthetic
ggccuuagcuacaggagag

SEQ ID NO: 3 - RNA, 19 nt, Artificial Sequence
  Synthetic
gaaaggauuuggcuacaaa

SEQ ID NO: 4 - RNA, 19 nt, Artificial Sequence
  Synthetic
acagcaaauuccaucgugu

SEQ ID NO: 5 - RNA, 19 nt, Artificial Sequence
  Synthetic
ggaaagacuguuccaaaaa

SEQ ID NO: 6 - RNA, 19 nt, Artificial Sequence
  Synthetic
cagggcggagacuucacca

SEQ ID NO: 7 - RNA, 19 nt, Artificial Sequence
  Synthetic
ugguuuacauguuccaaua

SEQ ID NO: 8 - RNA, 19 nt, Artificial Sequence
  Synthetic
guaugacaacagccucaag

SEQ ID NO: 9 - RNA, 19 nt, Artificial Sequence
  Synthetic
gcacauggauggagguucu

SEQ ID NO: 10 - RNA, 19 nt, Artificial Sequence
  Synthetic
gcagagagagcagauuuga

SEQ ID NO: 11 - RNA, 19 nt, Artificial Sequence
  Synthetic
gagguucucuggaucaagu

SEQ ID NO: 12 - RNA, 19 nt, Artificial Sequence
  Synthetic
gagcagauuugaagcaacu